(19) United States
(12) Patent Application Publication
(10) Pub. No.: US 2025/0152559 A1
(43) Pub. Date: May 15, 2025

(54) METHODS FOR TREATING MYC-AMPLIFIED CANCER

(71) Applicants: The Governing Council of the University of Toronto, Toronto (CA); McMaster University, Hamilton (CA)

(72) Inventors: Jason Moffat, Toronto (CA); Jose Rafael Montenegro Burke, Toronto (CA); Sheila K. Singh, Dundas (CA); William D. Gwynne, Hamilton (CA); Chitra Venugopal, Burlington (CA)

(21) Appl. No.: 18/944,172

(22) Filed: Nov. 12, 2024

Related U.S. Application Data
(60) Provisional application No. 63/547,962, filed on Nov. 9, 2023.

(51) Int. Cl. A61K 31/4196; A61P 35/00; G01N 33/50

(52) U.S. Cl. CPC A61K 31/4196; A61P 35/00; G01N 33/50; G01N 2800/52

(57) ABSTRACT

The present disclosure relates to the treatment of a myc-amplified cancer using a DHODH inhibitor. Also disclosed herein are combination therapies, compositions and kits for the treatment of a myc-amplified cancer comprising a DHODH inhibitor.

### RELATED APPLICATION

This application claims benefit of U.S. Provisional Patent Application Ser. No. 63/547,962 filed Nov. 9, 2023, incorporated herein by reference in its entirety.

### FIELD

The present disclosure relates generally to methods, uses and kits for treating cancer using DHODH inhibitors. In particular, the present invention pertains to the use of a DHODH inhibitor for the treatment of MYC-amplified cancers.

### INTRODUCTION

Central nervous system tumors recently surpassed leukemia to become the leading cause of pediatric cancer-related mortality in the United States¹. Medulloblastoma (MB), which describes a family of high-grade embryonal tumors of the postnatal cerebellum, is the most frequently diagnosed pediatric solid tumor². Whereas advances in the standard-of-care (SoC) for MB (surgery and craniospinal irradiation followed by multimodal chemotherapy) have improved the 5-year survival to 70%, many survivors, particularly infants, experience a reduced quality of life due to treatment-related neurotoxic sequelae³. Patients whose tumors fail to respond to first-line therapies invariably experience disease recurrence, and with a dismal one-year survivorship of 40%, recurrent MB remains ostensibly incurable^{4,5} These figures highlight an urgent need for novel therapeutic approaches to treat MB.

Integrated genomic analyses have stratified MB into four molecular subgroups⁶. In comparison to the other subgroups, Group 3 MB, particularly those harboring focal amplification of the oncogene MYC (G3MB-MYC), are poorly understood at the molecular level, and are associated with metastatic disease and high rates of recurrence. MB recurrence is thought to be driven by a subpopulation of brain tumor initiating cells (BTICs), which hijack DNA-repair⁷ and multidrug detoxification⁸ mechanisms to endure SoC, and exploit stem-like properties of self-renewal and proliferation to replenish tumor bulk^{9,10}. To better understand the dynamic molecular landscape of G3MB-MYC, a therapy-adapted mouse model that uses patient-derived orthotopic xenografts (PDOX) tissue was recently developed to recapitulate disease progression through treatment and relapse¹¹. Intriguingly, temporal transcriptomic profiling of tumor tissue from different disease stages of this model suggests that these tumor cells may reprogram notable metabolic pathways including oxidative phosphorylation (OXPHOS) and de novo lipogenesis. Metabolic reprogramming describes how tumor cells rewire their metabolism via genetic and epigenetic means to sustain macromolecular demands of proliferation, quench intracellular stressors such as free radicals, and potentiate oncogenic signaling programs that drive cancer progression¹²⁻¹⁵. Whereas several studies have identified metabolic enzymes as targetable drivers of cancer progression, to date, very few studies have investigated these targets in MB.

### SUMMARY

Cancer-selective therapeutic target selection requires an unbiased, comprehensive molecular profile of tumor cells in comparison to normal neural stem cells (NSCs), the proposed cell-of-origin of G3MB¹⁶. To this end, a genome-wide loss-of-function genetic screen using patient-derived tumor cells cultured under stem cell enrichment culture conditions was conducted^{9,10,17}. Therapeutic candidate genes were distilled by integrating the data with other genetic screens performed using NSCs derived from healthy nervous tissues¹⁸. Intriguingly, the leading-edge MB-selective essential genes (EGs) identified with this approach comprised several enzymes involved in purine and pyrimidine biosynthesis, spermidine metabolism, and the citric acid (TCA) cycle. The rewiring of these metabolic pathways was directly surveyed using unbiased mass spectrometry (MS)-based metabolomic profiling of G3MB-MYC and NSCs. Collectively, this multi-omic strategy revealed multiple metabolic vulnerabilities in G3MB-MYC tumor cells, including the genes encoding phosphogluconate dehydrogenase (PGD), spermidine synthase (SRM) and dihydroorotate dehydrogenase (DHODH).

DHODH emerged as a favorable target for G3MB-MYC given its druggability, tumor-selectivity and MB subgroup specificity. Metabolomic profiling showed that DHODH inhibitors act on-target, disrupting uridine metabolism and lipid homeostasis in patient-derived G3MB tumor cells. Mechanistically, DHODH inhibition evokes a metabolic stress response that attenuates the activity of mTORC1 and c-Myc and induces cell cycle arrest and apoptosis. Together, the data described herein provides a therapeutic paradigm for aggressive G3MB-MYC tumor cell phenotypes and highlight several compelling therapeutic targets for further development.

Accordingly, the present disclosure provides methods of treating a myc-amplified cancer comprising administering a dihydroorotate dehydrogenase (DHODH) inhibitor. This disclosure also relates to a method of selecting a therapy for treating a myc-amplified cancer.

One aspect of the disclosure includes a method of treating a myc-amplified cancer comprising administering a dihydroorotate dehydrogenase (DHODH) inhibitor, to a subject in need thereof, wherein the myc-amplified cancer is not acute myeloid leukemia (AML).

In an embodiment, the myc-amplified cancer is brain cancer, breast cancer, esophageal cancer, lymphoid cancer, myeloid cancer, lung cancer, kidney cancer, ovarian cancer, colorectal cancer or pancreatic cancer.

In a further embodiment, the myc-amplified cancer is myc-amplified Group 3 medulloblastoma (G3MB).

In a further embodiment, the myc-amplified cancer is a recurrent or a refractory cancer.

In an embodiment, the DHODH inhibitor is BAY2402234, Brequinar, PTC299, PTC868, or a combination thereof.

In another embodiment, the DHODH inhibitor is BAY2402234.

In another embodiment, the DHODH inhibitor is PTC299.

In another embodiment, the DHODH inhibitor is PTC868.

In another embodiment, the DHODH inhibitor is Brequinar.

In an embodiment, the DHODH inhibitor is permeable to the blood-brain barrier.

In an embodiment, the DHODH inhibitor is impermeable to the blood-brain barrier.

In an embodiment, the DHODH inhibitor is administered orally.

In an embodiment, the DHODH inhibitor is administered at a dose of about 5 mg/kg/day to about 10 mg/kg/day.

In an embodiment, the DHODH inhibitor is administered directly to the central nervous system, or optionally directly to the brain.

In an embodiment, the DHODH inhibitor is administered as a pharmaceutical composition comprising the DHODH inhibitor and a pharmaceutically acceptable carrier or diluent.

In an embodiment, the DHODH inhibitor is administered as a combination therapy.

In another embodiment, the combination therapy includes craniospinal irradiation and/or chemotherapy.

Another aspect of the disclosure is a method of treating a myc-amplified cancer in a subject comprising administering a dihydroorotate dehydrogenase (DHODH) inhibitor to a subject in need thereof, wherein the myc-amplified cancer has increased pyrimidine and/or purine metabolites relative to a non-cancerous control, wherein the cancer is not acute myeloid leukemia (AML).

In an embodiment, the method further comprises, prior to administration,
(a) obtaining a biopsy of the myc-amplified cancer; and
(b) detecting increased pyrimidine and/or purine metabolites in the biopsy.

In an embodiment, the pyrimidine metabolites include cytidine-5-monophosphate (CMP), uridine-5-monophosphate (UMP) or both.

In another embodiment, the purine metabolites include adenosine-5-monophosphate (AMP), guanosine-5-monophosphate (GMP) or both.

In an embodiment, the myc-amplified cancer is brain cancer, breast cancer, esophageal cancer, lymphoid cancer, myeloid cancer, lung cancer, kidney cancer, ovarian cancer, colorectal cancer or pancreatic cancer.

In another embodiment, the myc-amplified cancer is G3MB.

In a further embodiment, the myc-amplified cancer is a recurrent or a refractory cancer.

In an embodiment, the DHODH inhibitor is BAY2402234, Brequinar, PTC299, PTC868 or a combination thereof.

In another embodiment, the DHODH inhibitor is BAY2402234.

In another embodiment, the DHODH inhibitor is PTC299.

In another embodiment, the DHODH inhibitor is PTC868.

In another embodiment, the DHODH inhibitor is Brequinar.

In an embodiment, the DHODH inhibitor is permeable to the blood-brain barrier.

In an embodiment, the DHODH inhibitor is impermeable to the blood-brain barrier.

In an embodiment, the DHODH inhibitor is administered orally.

In an embodiment, the DHODH inhibitor is administered at a dose of about 5 mg/kg/day to about 10 mg/kg/day.

In an embodiment, the DHODH inhibitor is administered directly to the central nervous system, or optionally directly to the brain.

In an embodiment, the DHODH inhibitor is administered as a pharmaceutical composition comprising the DHODH inhibitor and a pharmaceutically acceptable carrier or diluent.

In an embodiment, the DHODH inhibitor is administered as a combination therapy.

In another embodiment, the combination therapy includes craniospinal irradiation and/or chemotherapy.

Yet another aspect of the disclosure is a method of selecting a therapy for treating a myc-amplified cancer in a subject comprising:
(a) obtaining a biopsy of the myc-amplified cancer;
(b) detecting increased pyrimidine and/or purine metabolites in the biopsy; and
(c) selecting a dihydroorotate dehydrogenase (DHODH) inhibitor for treating the subject when there is an increased level of pyrimidine and/or purine metabolites in the biopsy, wherein the cancer is not acute myeloid leukemia (AML).

In an embodiment, the pyrimidine metabolites include cytidine-5-monophosphate (CMP), uridine-5-monophosphate (UMP) or both.

In another embodiment, the purine metabolites include adenosine-5-monophosphate (AMP), guanosine-5-monophosphate (GMP) or both.

In an embodiment, the myc-amplified cancer is brain cancer, breast cancer, esophageal cancer, lymphoid cancer, myeloid cancer, lung cancer, kidney cancer, ovarian cancer, colorectal cancer or pancreatic cancer.

In another embodiment, the myc-amplified cancer is G3MB.

In a further embodiment, the myc-amplified cancer is a recurrent or a refractory cancer.

In an embodiment, the DHODH inhibitor is BAY2402234, Brequinar, PTC299, PTC868, or a combination thereof.

In another embodiment, the DHODH inhibitor is BAY2402234.

In another embodiment, the DHODH inhibitor is PTC299.

In another embodiment, the DHODH inhibitor is PTC868.

In another embodiment, the DHODH inhibitor is Brequinar.

In an embodiment, the DHODH inhibitor is permeable to the blood-brain barrier.

In an embodiment, the DHODH inhibitor is impermeable to the blood-brain barrier.

The preceding section is provided by way of example only and is not intended to be limiting on the scope of the present disclosure and appended claims. Additional objects and advantages associated with the compositions and methods of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional advantages objects and embodiments are expressly included within the scope of the present disclosure. The publications and other materials used herein to illuminate the background of the disclosure, and in particular cases, to provide additional details respecting the practice, are incorporated by reference, and for convenience are listed in the appended reference section.

### DRAWINGS

Further objects, features and advantages of the disclosure will become apparent from the following detailed description taken in conjunction with the accompanying figures showing illustrative embodiments of the disclosure, in which:

FIGS. 1A-C show the integrated functional genomics and metabolomic analyses that identified targetable metabolic vulnerabilities in MYC-amplified group 3 medulloblastoma. FIG. 1A Schematic of method used to perform genome-wide loss of function CRISPR-Cas9 screen using SU_MB002 cells transduced with the TKOv3 library. EG=essential gene. NGS=next-generation sequencing. FIG. 1B Schematic of bioinformatic pipeline that filtered PAN-EGs (top). The highest ranking (FDR) gene ontologies (biological processes) associated with top SU_MB002 EGs (middle). Venn diagram of 374 SU_MB002-selective EGs after filtering EGs (BF>5) identified in either CB66 or U5 NSC. Heatmap showing mean change in sgRNA abundance of the top-ranking SU_MB002-exclusive EGs in each of the screens (log 2-transformed fold changes). The genes annotated with enzymatic function in metabolism are in bold (bottom). FIG. 1C Schematic of MS-based metabolomic profiling (top), heatmap of differentially abundant metabolites detected in HD-MB03, MED411FHTC, and SU_MB002, as compared to NSC194, NSC197, and NSC201FT (normalized LC-MS peak areas). n=5 independent replicates. Significance determined via Benjamini, Krieger and Yekutieli (Q≥1%) (bottom).

FIGS. 2A-N show attenuation of key metabolic pathways selectively targeted BTICs in MYC-amplified group 3 medulloblastoma. FIG. 2A Immunoblot confirmation of DHODH and PGD KO in SU_MB002. FIG. 2B Assessment of tumor cell growth kinetics measured by time-course PrestoBlue™ reduction cell viability assays in DHODH, PGD (2 sgRNA tested per gene) and AAVS1 KO SU_MB002 tumor cells. Comparison of cell viability after 120 hours were made via One-way ANOVA and post-hoc Tukey's test, **** p<0.0001, n=4 independent replicates. FIG. 2C Representative phase-contrast microscopy images of SU_MB002 cells harboring AAVS1, PGD and DHODH KO. Scale=200 μm FIG. 2D Quantification of tumor spheres formed by DHODH-KO A, PGD-KO A and AAVS1 KO SU_MB002 tumor cells. One-way ANOVA and post-hoc Tukey's test **** p<0.0001, n=4 independent replicates. FIG. 2E Assessment of tumor cell growth kinetics measured by time-course PrestoBlue™ reduction cell viability assays in DHODH-KO A, PGD-KO A and AAVS1 KO HD-MB03 tumor cells. FIG. 2F Quantification of tumor spheres formed by DHODH-KO A, PGD-KO A and AAVS1 KO HD-MB03 tumor cells. FIG. 2G Immunoblot confirmation of KO of DHODH-KO A and PGD-KO A in NSC197 cells. FIG. 2H Assessment of cell viability of DHODH-KO A, PGD-KO A, and AAVS1 KO NSC197 cells expressed as percent residual PrestoBlue™ reduction (percent AAVS1). FIG. 2I Assessment of cell viability of SU_MB002 tumor cells treated with PGD inhibitor S3 (20 μM) or its vehicle (0.5% DMSO). FIGS. 2J-K Dose-response curves of cell viability after a 72-hour treatment with BAY2402234 (FIG. 2J) and Brequinar (BQR; FIG. 2K). Cell viability (residual PrestoBlue™ reduction) normalized to vehicle-treated cells. IC₅₀ calculations made via nonlinear regression. FIG. 2L Kaplan-Meier survival analysis of mice xenografted with DHODH and AAVS1 KO tumor cells (HD-MB03 and SU_MB002). Log-ranks test ** p=0.0053, n=5-6 mice. FIG. 2M Representative H&E-stained sections of brains isolated from time-matched specimens. FIG. 2N Tumor area (mm²) of time-matched brain tissue sections. Paired t-test; * p=0.01; *** p=0.001, n=3-4 mice. All error bars represent standard error of the mean calculated across four technical replicates.

FIGS. 3A-L show that DHODH sustained the transcriptional activity of c-Myc and drove cell-cycle progression in MYC-amplified G3MB. FIG. 3A UMAP representation of transcriptomics clusters (0-6) identified in 6,327 G3MB cells from 8 surgical specimens. FIGS. 3B-C Pyrimidine metabolism is correlated with (FIG. 3B) MYC target activity and (FIG. 3C) cellular entropy. FIG. 3D Transcript expression of pyrimidine biosynthesis genes in medulloblastoma (n=22) and non-tumor brain tissues (n=13). One-way ANOVA and post-hoc Tukey's test (**** p<0.0001; ** p=0.003). FIG. 3E Kaplan-Meier survival curve of G3MB patients stratified by DHODH or UPP1 transcript expression. Upper and lower quartile separation (n=28). Log-rank test ** p=0.008. FIG. 3F Volcano plot of DEGs (Limma) in DHODH KO SU_MB002 cells log 2 fold-change (log FC) expression compared to AAVS1 KO. FIG. 3G Enrichment of HALLMARK pathways associated with DHODH KO determined by GSEA. NES=normalized enrichment score. FIG. 3H Dot blot (left) of significant phosphoproteins whose abundance changed with BAY treatment (48 h) in comparison to vehicle. Bar plot (right) with corresponding mean pixel densities of abundant phosphoproteins. * p<0.01 one-way ANOVA and Tukey's test, n=2 independent replicates. FIG. 3I Time-course Western blots showing expression of p-ERK, total ERK, p-4EBP1,4EBP1, cleaved caspase 3 and GAPDH. FIG. 3J Densitometry (ImageStudio Lite) showing caspase 3 cleavage (top), ERK phosphorylation and expression (middle), and 4EBP1 phosphorylation and expression (bottom) with respect to time-course treatment of SU_MB002 cells with BAY. Expression in BAY-treated cells normalized to that of the vehicle. FIGS. 3K-L Pie charts showing cell cycle phase (DNA content, PI) (FIG. 3K) and Annexin V positivity versus viability (DNA content, PI) (FIG. 3L) in DHODH KO or BAY-treated SU_MB002 cells, in comparison to respective controls (percentages derived from FACS analysis shown in FIG. 8N-O).

FIGS. 4A-G show that DHODH inhibitors exerted on-target therapeutic effects by altering the metabolome and lipidome of MYC-amplified group 3 medulloblastoma in a uridine-dependent manner. FIG. 4A Heatmap of differentially abundant metabolites (top left), triglycerides (top right, bottom right), and ether-linked PE species detected in DHODH-deficient SU_MB002 (DHODH KO vs AAVS1 KO and BAY2402234-treated vs vehicle-treated), n=4 independent replicates. Map of de novo pyrimidine biosynthesis is displayed (bottom left); Significance determined via Benjamini, Krieger and Yekutieli (Q≥1%). Data are log 2 fold changes. FIG. 4B Western blot profiles showing expression of O-GlcNAc-linked proteins, c-Myc, and GAPDH. FIG. 4C Schematic showing O-GlcNAc stabilization of c-Myc. FIG. 4D Dose-response curves of SU_MB002 tumor cells treated with BAY2402234 or BQR in culture media supplemented with uridine hydrochloride (100 μM) or vehicle (H₂O), n=4 independent experiments. FIG. 4E Representative phase-contrast images of BAY2402234-treated (12 nM) SU_MB002 tumor cells with or without uridine rescue. Scale=200 μm FIG. 4F Time-course cell-growth assays of AAVS1 and DHODH KO SU_MB002 cells grown in cell culture supplemented with or without uridine, n=4 independent experiments. FIG. 4G Cell viability of SU_MB002 AAVS1 KO and DHODH KO tumor cells after 5 days of growth with or without uridine. One-way ANOVA and Tukey's test, **** p<0.0001. All error bars represent standard error of the mean calculated across four technical replicates.

FIGS. 5A-K show that pharmacological inhibition of DHODH demonstrated efficacy in a preclinical PDOX model of MYC-amplified group 3 medulloblastoma. FIG. 5A Permeability coefficients (log PE) for brequinar (BQR), BAY2402234 and caffeine. One-way ANOVA and Tukey's test, ** p<0.01, n=4 independent experiments. FIG. 5B Treatment regimens R1 and R2. 10-day treatment periods were interrupted with a 4-day period of no treatment. FIG. 5C IVIS (in vivo imaging system) bioluminescence signals of vehicle and R1-treated mice before treatment and at vehicle-endpoint. FIG. 5D PDOX growth determined by IVIS bioluminescence imaging (total flux in photons per second). FIG. 5E Tumor burden comparisons between vehicle-, R1- and R2-treated mice at vehicle endpoint. One-way ANOVA, post-hoc Tukey's test, *** p=0.006, *** p=0.0007. FIG. 5F Kaplan-Meier survival analysis of vehicle-, R1- and R2-treated mice. Log-rank test, *** p=0.0029 (R1); *** p=0.0013 (R2). FIG. 5G Flank tumor growth determined by IVIS bioluminescence imaging (total flux in photons per second). FIG. 5H LC-MS quantified BAY2402234 concentrations in plasma and brain tissues (n=5). T-test, **** p<0.0001. FIG. 5I Ratio of BAY2402234 brain concentration relative to plasma concentration in each mouse. FIGS. 5J-K Heatmap of metabolites enriched/depleted in BAY-treated brain tissues (FIG. 5J) and flank xenograft tissues (FIG. 5K). Values are log₂ FC relative to vehicle-treated mice. All error bars represent standard error of the mean calculated across four technical replicates.

FIGS. 6A-K show G3MB tumor cells harbored genetic dependencies in metabolic pathways [Related to FIG. 2]. FIG. 6A Comparison of gene effect of G3MB context EGs for brain tumor cell lines compared to G3MB cell lines. Significance determined via unpaired t-test (** p<0.01). FIG. 6B Assessment of tumor cell growth kinetics measured by time-course PrestoBlue™ reduction cell viability assays in SRM (2 sgRNA tested per gene) and AAVS1 KO SU_MB002 tumor cells. FIG. 6C Comparison of cell viabilities after 120 h were made via one-way ANOVA and post-hoc Tukey's test, **** p<0.0001, n=4 independent replicates. FIG. 6D Quantification of number of tumorspheres formed by SRM and AAVS1 KO SU_MB002 tumor cells. One-way ANOVA and post-hoc Tukey's test **** p<0.0001, n=4 independent replicates. FIG. 6E PTC299 dose-response in SU_MB002 and NSC197 cells. FIG. 6F MBT375 analyzed with Nanostring subtyping MB panel. Heatmap shows log 2 normalized expression counts. FIG. 6G Image of MBT375 PDX in mouse cerebellum. FIG. 6H Images of H&E-stained sections of MBT375 PDX. Scale: 200 μm. FIG. 6I BAY2402234 dose-response in ICB1299 and MBT375 tumor cells. FIG. 6J Section of SU_MB002 PDOX stained with anti-rabbit secondary antibody only (negative control). FIG. 6K Sections of PDOX from DHODH KO and AAVS1 KO probed for DHODH expression. Scale: 50 μm.

FIGS. 7A-O shows that EGR1 activity was anticorrelated with de novo pyrimidine biosynthesis in G2MB [Related to FIG. 3]. FIG. 7A UMAP representation of cell cycle phase of transcriptomics clusters 0-6 identified in 6,327 G3MB cells from 8 surgical specimens. FIGS. 7B-F Gene expression programs stratified by cluster. Identified in the left-most cluster are proliferative cell clusters (3 and 5) enriched in pyrimidine metabolism, MYC targets and high cellular entropy (CytoTrace scores). FIG. 7G EGR1 gene expression (top left) and regulon activity (bottom left) overlaid on UMAP representation of G3MB sc-RNAseq profiles (6,327 cells from 8 surgical specimens). Correlation between regulon activity and gene expression (top right). EGR1 regulon activity in SEURAT clusters 0-6 (bottom right). FIG. 7H EGR1 regulon-associated pathways enriched in G3MB tumor cells. FIG. 7I MYC gene expression (top left) and regulon activity (bottom left) overlaid on UMAP representation of G3MB sc-RNAseq profiles. Correlation between regulon activity and gene expression (top right) and cluster-specific regulon activities (bottom right) are shown. FIG. 7J MYC regulon-associated pathways enriched in G3MB tumor cells. FIG. 7K Heatmap showing relationship (rho) between EGR1 and c-Myc gene expression, regulon activity, and pyrimidine biosynthesis. FIG. 7L Scatter plots showing correlations between MYC and EGR1 regulon (left) pyrimidine metabolism and MYC regulon (middle) and pyrimidine metabolism and EGR1 regulon (right). FIG. 7M Schematic definition of CpG-associated genomic features (top) and rug plots of CpG enrichment scores for EGR1 regulon-associated genes (black) and randomly perturbed genes (grey, n=1000 iterations) (bottom). FIG. 7N Heatmap showing the DMPs at promotor islands for BAY vs DMSO treatment at 72 h using 10% FDR and log FC>|0.1| cut-off. Z-score-normalized beta values for all samples at 48 h and 72 h are shown. DMPs within promotor islands considering TSS1500, TSS200, 5′UTR and 1st exon as promotor regions. FIG. 7O Q-values of top transcription factor ChIP-seq datasets (ranked by Enrichr combined score) that associated with genes from promoter island DMPs, identified using Enrichr analysis ENCODE TF ChIP-seq 2015.

FIGS. 8A-P show that de novo pyrimidine biosynthesis transcript and protein expression were G3MB-specific and associated with c-Myc expression and cell cycle progression [Related to FIG. 3]. FIGS. 8A-B UMAP representation of MB scRNA-seq profiles. Individual tumors (FIG. 8A) and MB subtypes (FIG. 8B) are indicated, and each node represents an individual cell. FIGS. 8C-D scRNA-seq-based MYC metagene activity overlaid on UMAP (FIG. 8C) and compared across MB subtypes in boxplots (FIG. 8D). FIGS. 8E-F De novo pyrimidine biosynthesis metagene activity overlaid on UMAP (FIG. 8E) and compared across MB subtypes in boxplots (FIG. 8F). FIG. 8G UMAP representation of MB proteomic profiles. Each node represents an individual patient tumor, and MB subtypes are indicated. G3a represents the MYC-activated form of MB whereas G3b represents the known G3/4 continuum state. FIGS. 8H-I MYC (FIG. 8H) and de novo pyrimidine biosynthesis (FIG. 8I) metagene scores compared across MB subtypes in boxplots. FIGS. 8J-K Differential expression of genes (FIG. 8J) and proteins (FIG. 8K) associated with de novo pyrimidine biosynthesis in G3 vs. other subtype MBs [FIGS. 8J and 8K (left)] or G3a vs. G3b (FIG. 8K, right). Differences evaluated using Wilcoxon test, and p values adjusted using Benjamini-Hochberg procedure. 5% FDR threshold used to determine significance. Shaded area with solid line represents average log fold change (LFC)±95% confidence interval between indicated groups. For FIGS. 8D, 8F, 8H and 8I, comparison across subtypes was assessed using one-way ANOVA. FIG. 8L Kaplan-Meier survival analysis for all MB subtypes stratified according to upper and lower quartile mRNA expression of CAD, DHODH and UMPS. Log-rank test, p-value displayed on graph. FIG. 8M Western blots shown for p-4EBP1, cleaved caspase 3 and GAPDH in HD-MB03 treated with BAY (IC50) or vehicle. FIGS. 8N-O FACS data corresponding to percentages displayed in FIG. 3K-L. FIG. 8P Western blots (bottom) showing EGR1 expression. Graphs (top) show expression relative to loading control protein. Unpaired t-test, *p=0.01, n=2 experimental replicates.

FIGS. 9A-T shows that DHODH essentiality was associated with MYC-expressing tumor types in genetic screens [Related to FIG. 4]. FIG. 9A DHODH gene effect across cancer cell lines grouped by lineage. FIG. 9B Comparison of DHODH, CAD and UMPS gene effect for brain tumor cell lines compared to G3MB cell lines. Significance determined via unpaired t-test (** p<0.01). Correlation of gene effect of FIGS. 9C-H DHODH, FIGS. 91-N CAD and FIGS. 9O-T UMPS compared with (FIGS. 9C-E, 9I-K, 9O-Q) MYC gene effect and (FIGS. 9F-H, 9L-N, 9R-T) MYC gene expression for all cancer cell lines ((FIGS. 9C, 9I, 9O) n=1086; (FIGS. 9F, 9L, 9R) n=1005), brain cancer cell lines ((FIGS. 9D, 9J, 9P) n=78; (FIGS. 9G, 9M, 9S) n=70) and medulloblastoma cell lines ((FIGS. 9E, 9K, 9Q; 9H, 9N, 9T) n=7). Linear model was fit and Pearson correlation coefficient and p-value are shown. Plots were generated using R 4.1.2 and package ggplot2. Data was available for the following medulloblastoma cell lines: D283MED, D341, DAOY, ONS76, D425, D458 and UW228. Data was obtained from DepMap portal (CRISPR DepMap 22Q2 Public+Score Chronos; Expression 22Q2 Public, log 2(TPM+1)).

FIGS. 10A-H shows that DHODH was a therapeutic target in MYC-G3MB [Related to FIG. 5]. FIGS. 10A-B Raw IVIS flux values from HD-MB03 PDOX (FIG. 10A) and flank (FIG. 10B) xenograft. FIG. 10C Kaplan-Meier survival analysis of vehicle and BAY-treated mice. Log-rank test, **p<0.01. FIG. 10D Tumor area quantification in time-matched brains resected from SU_MB002 PDOX-bearing mice treated with BAY or the vehicle (determined by H&E and measured via ImageJ). Unpaired t-test, **p<0.01. FIG. 10E Bioluminescence flux values of vehicle-treated, and BAY-treated mice at endpoint. Unpaired t-test, *p<0.05. FIG. 10F Mouse body weight throughout the course of BAY2402234 R1. FIG. 10G Images of biological duplicate flank xenografts at endpoint treated with vehicle (top two rows) or BAY (bottom two rows). Sections from formalin fixed paraffin embedded samples were examined via H&E staining, and immunohistochemistry staining of Ki67 proliferation marker. FIG. 10H H&E-stained sections of time-matched PDOX resected from BAY- and vehicle-treated mice (left), IHC staining of EGR1, black arrows point to faintly positive cells and red arrows to highly positive cells (middle), IHC staining of p-4EBP1 (right). Scale: 4 mm.

### DESCRIPTION OF VARIOUS EMBODIMENTS

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature described herein may be combined with any other feature or features described herein.

### I. Definitions

As used herein, the following terms may have meanings ascribed to them below. However, it should be understood that other meanings that are known or understood by those having ordinary skill in the art are also possible, and within the scope of the present disclosure. In the case of conflict, the present specification, including definitions, will control. In addition, the materials, methods, and examples are illustrative only and not intended to be limiting. All publications, patent applications, patents, and other references mentioned herein are incorporated by reference in their entirety.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Ranges from any lower limit to any upper limit are contemplated.

The term “about” as used herein may be used to take into account experimental error and variations that would be expected by a person having ordinary skill in the art. For example, “about” may mean plus or minus 10%, or plus or minus 5% of the indicated value to which reference is being made.

As used herein the singular forms “a”, “an”, and “the” include plural references unless the context clearly dictates otherwise.

The phrase “and/or,” as used herein, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

As used herein, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, in certain methods described herein that include more than one step or act, the order of the steps or acts of the method is not necessarily limited to the order in which the steps or acts of the method are recited unless the context indicates otherwise.

### II. Methods and Uses

As shown herein, dihydroorotate dehydrogenase (DHODH) inhibitors are efficacious in treating myc-amplified cancers. The present inventors have shown that myc-amplified cancers have increased levels of pyrimidine and/or purine metabolites and are candidates for treatment with DHODH inhibitors.

Accordingly, an aspect of the present disclosure is a method of treating a myc-amplified cancer comprising administering a DHODH inhibitor to a subject in need thereof. Also provided is use of a DHODH inhibitor for treating a myc-amplified cancer in a subject in need thereof. Further provided is use of a DHODH inhibitor in the manufacture of a medicament for treating a myc-amplified cancer in a subject in need thereof. Even further provided is a DHODH inhibitor for use in the treatment of a myc-amplified cancer in a subject in need thereof. In an embodiment, the myc-amplified cancer is not Acute Myeloid Leukemia (AML).

Another aspect of the disclosure includes a method of treating a myc-amplified cancer, wherein the myc-amplified cancer is not AML, in a subject comprising treating the subject with a DHODH inhibitor, wherein a biopsy of the myc-amplified cancer in the subject has been shown to have an increased level of pyrimidine and/or purine metabolites compared to a non-cancerous control. In an embodiment, the method includes first obtaining a biopsy of the myc-amplified cancer from the subject and detecting the level of pyrimidine and/or purine metabolites in the biopsy compared to a non-cancerous control. Also provided is use of a DHODH inhibitor for treating a myc-amplified cancer in a subject, wherein a biopsy of the myc-amplified cancer in the subject has been shown to have an increased level of pyrimidine and/or purine metabolites compared to a non-cancerous control. Further provided is use of a DHODH inhibitor in the manufacture of a medicament for treating a myc-amplified cancer in a subject, wherein a biopsy of the myc-amplified cancer in the subject has been shown to have an increased level of pyrimidine and/or purine metabolites compared to a non-cancerous control. Even further provided is a DHODH inhibitor for use in the treatment of a myc-amplified cancer in a subject, wherein a biopsy of the myc-amplified cancer in the subject has been shown to have an increased level of pyrimidine and/or purine metabolites compared to a non-cancerous control.

A further aspect is a method of selecting a therapy for treating a myc-amplified cancer, wherein the myc-amplified cancer is not AML, in a subject comprising detecting the levels of pyrimidine and/or purine metabolites in a biopsy from the subject and selecting a DHODH inhibitor for treating the subject when there is an increased level of pyrimidine and/or purine metabolites in the biopsy compared to a non-cancerous control.

The term “non-cancerous control”, as used herein refers to a non-cancerous comparative tissue taken from a cancer-free subject, or a specific value or dataset that can be used to prognose or classify the value e.g., pyrimidine and/or purine level or reference pyrimidine and/or purine value obtained from a test sample or samples associated with a known outcome. In one embodiment, the dataset may be obtained from samples of a group of subjects known to be non-cancerous and have non-cancerous levels of pyrimidine and/or purine metabolites. In another embodiment, the dataset may be obtained from samples of a group of subjects known to not possess amplification of the MYC oncogene. The level of the pyrimidine and/or purine metabolites in the dataset can be used to create a “control value” that is used in testing samples from new patients. A control value is obtained from the historical pyrimidine and/or purine metabolite levels for a patient or pool of patients with a known outcome.

In an embodiment, the method further comprises obtaining a biopsy of the myc-amplified cancer. The term “biopsy” as used herein refers to the removal of a piece of tissue or a sample of cells from the body for examination.

The term “pyrimidine” as used herein refers to a heterocyclic aromatic, six-membered ring structure composed of two nitrogen atoms and four carbon atoms. Pyrimidines include numerous derivatives, such as the nitrogenous base cytosine, thymine and uracil, the nucleosides uridine, thymidine and cytosine and the nucleotides cytidine monophosphate (CMP) and uridine monophosphate (UMP).

The term “purine” as used herein refers to a heterocyclic aromatic compound that consists of two fused ring structures, namely the 6-membered pyrimidine and imidazole. Purines include numerous derivatives, such as the nitrogenous bases adenine and guanine, the nucleosides adenosine and guanosine, and the nucleotides adenosine monophosphate (AMP) and guanosine monophosphate (GMP).

In an embodiment, the pyrimidine and/or purine metabolites comprise at least one of adenosine-5-monophosphate (AMP), cytidine-5-monophosphate (CMP), uridine-5-monophosphate (UMP) and guanosine-5-monophosphate (GMP). In one embodiment the pyrimidine metabolite comprises UMP.

As used herein, the term “cancer” refers to one of a group of diseases caused by the uncontrolled, abnormal growth of cells that can spread to adjoining tissues or other parts of the body. Cancer cells can form a solid tumor, in which the cancer cells are massed together, or exist as dispersed cells. The cancer may be of any stage (early stage, locally advanced, or advanced), and may optionally be metastatic cancer, relapsed cancer, refractory cancer and/or cancer with acquired chemoresistance. In an embodiment, the cancer is advanced cancer, metastatic cancer, relapsed cancer, refractory cancer, and/or cancer with acquired chemoresistance. As used herein, “refractory cancer” means a cancer that is intrinsically refractive to treatment, i.e. the cancer is not sensitive to a particular chemotherapy upon first exposure to the chemotherapy. As used herein “relapsed cancer” means a cancer that relapses and begins to grow again upon discontinuation of a particular chemotherapy. As used herein, “acquired chemoresistance” is an active process that develops within a cancer during treatment that makes the cancer less or entirely non responsive to a given chemotherapy.

The term “myc-amplified cancer” as used herein refers to, cancers harboring focal amplification of the oncogene MYC.

As used herein, MYC refers to the human gene, Myc refers to the mouse gene. c-Myc refers to the human protein.

In an embodiment, the myc-amplified cancer is brain cancer, ovarian cancer, esophageal cancer, lymphoid cancer, myeloid cancer, kidney cancer, lung cancer including squamous lung cancer, breast cancer, prostate cancer, colorectal cancer or pancreatic cancer.

In embodiment, the myc-amplified cancer is brain cancer.

In one embodiment the brain cancer is medulloblastoma.

In a particular embodiment, the MYC-amplified medulloblastoma is Group 3 medulloblastoma (G3MB).

In an embodiment, the myc-amplified cancer is a recurrent or a refractory cancer.

As used herein, the term medulloblastoma describes a high-grade embryonal tumor of the post-natal cerebellum. Medulloblastoma is the most common pediatric malignant brain cancer. G3MB largely occurs in infants and children, making up approximately 25% of all medulloblastoma cases. It occurs in males twice as often as in females. G3MB patients have a greater incidence of high risk clinical and molecular features including young age, metastases, large-cell/anaplastic histology and MYC amplification. G3MB patients require more intense treatment, and more often relapse and have poor prognosis. A subset of G3MB tumors exhibits overexpression of transcription factors of the growth factor independent 1 family as a result of DNA structural changes that transform the genes encoding these factors almost into super-enhancers. Transforming growth factor beta signaling pathways are activated in G3MB. MYC oncogene amplification is the most validated prognostic marker, conferring poor prognosis.

The term “cancer cell” refers to a cell characterized by uncontrolled, abnormal growth and the ability to invade another tissue or a cell derived from such a cell. Cancer cells include, for example, a primary cancer cell obtained from a subject with cancer or cell line derived from such a cell.

The term “subject” as used herein includes all members of the animal kingdom including mammals, and suitably refers to humans. Optionally, the term “subject” includes mammals that have been diagnosed with cancer or are in remission. In one embodiment, the term “subject” refers to a human having, or suspected of having, cancer.

The term “subject in need thereof” refers to a subject that could benefit from the method(s) or treatment(s) described herein, and optionally refers to a subject with myc-amplified cancer.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease (e.g. maintaining a subject in remission), preventing disease or preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein can also mean reducing the cancer burden in a subject.

The phrase “cancer burden” refers to the quantum of cancer cells or cancer volume in a subject. Reducing cancer burden accordingly refers to reducing the number of cancer cells or the cancer volume in a subject.

The term “palliating” a disease or condition means that the extent and/or undesirable clinical manifestations of a disease, disorder or condition are lessened and/or time course of the progression is slowed or lengthened, as compared to not treating the disorder.

As used herein, the term “DHODH” refers to dihydroorotate dehydrogenase, an enzyme that catalyzes de novo pyrimidine biosynthesis and can be from any organism or source. The nucleotide and amino acid sequence of human DHODH can be found at, for example GenBank Accession No. M94065.1 (NP_001352.2) or UniProt Q02127.

As used herein the term “inhibitor” refers to without limitation, a compound that reduces, decreases or otherwise blocks expression or activity of its target, and includes, without limitation, nucleic acids, antisense oligonucleotide molecules (antisense nucleic acid molecules), DNA, RNA, shRNA, siRNA, proteins, protein mimetics, peptides, peptidomimetics, antibodies (and fragments thereof), aptamers, small molecules, chemicals, analogs that mimic the binding site of an enzyme, receptor, or other protein, e.g., that is involved in signal transduction, gene editing agents, therapeutic agents, pharmaceutical compositions, drugs, other substances directed at the target expression or activity and combinations of these.

In an embodiment, the DHODH inhibitor is a compound as disclosed in U.S. Pat. No. 10,815,215, herein incorporated by reference in its entirety.

In an embodiment, the DHODH inhibitor is BAY2402234, Brequinar, PTC299 or PTC868.

In an embodiment, the DHODH inhibitor is BAY2402234.

BAY2402234, as used herein, refers to the compound with the following structure:

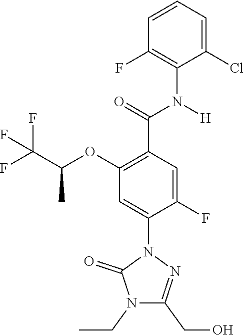 [SMILES: CCn1c(CO)nn(-c2cc(O[C@@H](C)C(F)(F)F)c(C(=O)Nc3c(F)cccc3Cl)cc2F)c1=O]

or a pharmaceutically acceptable salt or solvate thereof.

In an embodiment, the DHODH inhibitor is Brequinar.

Brequinar, as used herein, refers to the compound with the following structure:

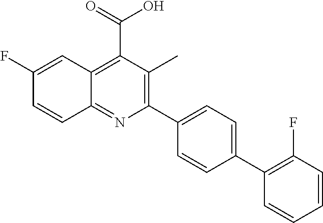 [SMILES: Cc1c(-c2ccc(-c3ccccc3F)cc2)nc2ccc(F)cc2c1C(=O)O]

or a pharmaceutically acceptable salt or solvate thereof.

In an embodiment, the DHODH inhibitor is PTC299, also known as emvodostat.

PTC299, as used herein, refers to the compound with the following structure:

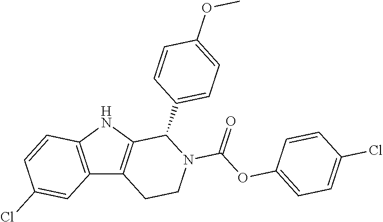 [SMILES: COc1ccc([C@H]2c3[nH]c4ccc(Cl)cc4c3CCN2C(=O)Oc2ccc(Cl)cc2)cc1]

or a pharmaceutically acceptable salt or solvate thereof.

In an embodiment, the DHODH inhibitor is PTC868.

PTC868, as used herein, is an optimized version of PTC299 which improves blood-brain barrier permeability, or a pharmaceutically acceptable salt or solvate thereof.

Pharmaceutically acceptable salts may include, for example, salts prepared using an organic or inorganic acid such as hydrochloric, formic, maleic, benzoic, hydrobromic, acetic, malonic, glucuronic, phosphoric, methanesulfonic, glutamic, fumaric, sulfuric, tosic, aspartic, nitric, tartaric, lactic, pyruvic, or mucic acid.

In an embodiment, the DHODH inhibitor, or pharmaceutically acceptable salt or solvate thereof, is prepared in various forms and/or polymorphs, including amorphous phase, crystalline forms, milled forms and nano-particulate forms, and or mixtures thereof.

As used herein, “solvate” means a solvation product of a compound, formed for example as a combination of solvent molecules, such as water, an alcohol, or other polar organic solvent, with molecules or ions of the solute compound. Suitable solvents are physiologically tolerable at the dosage administered, and may include, without limitation, methanol, ethanol, n-propanol, isopropanol, n-butanol, isobutanol, t-butanol, ethyl acetate and other lower alkanols, glycerine, acetone, Ethoxy ethanol dichloromethane, Dimethyl sulphoxide (DMSO), Dimethyl acetate (DMA), dimethyl formamide (DMF), isopropyl ether, methyl ethyl ketone, acetonitrile, toluene, N-methylpyrrolidone (NMP), tetrahydrofuran (THF), tetrahydropyran, water, other cyclic mono-, di- and tri-ethers, polyalkylene glycols ‘(e. g., polyethylene glycol, polypropylene glycol, propylene glycol) and mixtures thereof in suitable proportions.

In an embodiment the DHODH inhibitor is permeable to the blood-brain barrier and is optionally administered or used orally.

In an embodiment the DHODH inhibitor is impermeable to the blood-brain barrier and is administered or suitable for use directly to the central nervous system, or optionally directly to the brain.

The term “administered” or “administering” as used herein means administration of a therapeutically effective amount of a compound or composition of the disclosure to a cell either in cell culture or in a subject.

Examples of suitable dosage ranges for DHODH inhibitors may include for example up to 10 mg/kg. BAY2402234 is available as a crystalline solid. BAY2402234 has been administered to patients via oral administration (NCT 05061251). Daily doses of about 5 mg/kg body weight to about 10 mg/kg body weight are shown herein to be effective in a mouse model. Suitable dosages in humans can be estimated for example by calculating the human equivalent dose (HED) using for example the methods of Nair & Jacob (2016) 78. Accordingly, doses of BAY2402234 include, for example about 0.4065 mg/kg body weight to about 0.8130 mg/kg body weight. Examples of suitable dosage ranges for DHODH inhibitors can be determined by the skilled person. For example, Brequinar may be administered at a dosage of about 200 mg/m² to 500 mg/m² (NCT03760666). For example, PTC299 may be administered at a dosage of about 0.6 mg/kg body weight to about 1.2 mg/kg body weight, or about 1.2 mg/kg body weight, or about 1.5 mg/kg body weight or about 2.0 mg/kg body weight (NCT01158300). For example, PTC868, the optimized version of PTC299, may be administered in mice at a dosage of up to 10 mg/kg body weight. Other dosages and intervals are specifically contemplated herein. Optionally, lower doses of DHODH inhibitors may be used in combination with other therapies.

Such dosages may be administered according to any suitable schedule for example daily, weekly, bi-weekly, monthly, or any other suitable frequency, depending on the given drug combination, formulation(s) and/or route(s) of administration, stage of disease, and/or subject-specific factors. Examples of suitable dosing schedules can be determined by the skilled person.

The composition may be formulated for use or prepared for administration to a subject using pharmaceutically acceptable formulations known in the art. Conventional procedures and ingredients for the selection and preparation of suitable formulations are described, for example, in Remington's Pharmaceutical Sciences (2003—20th edition) and in The United States Pharmacopeia: The National Formulary (USP 24 NF19) published in 1999. The term “pharmaceutically acceptable” means compatible with the treatment of animals, in particular, humans.

The term “dosage form” as used herein refers to the physical form of a dose for example comprising a compound of the disclosure, and includes without limitation injectable dosage forms, including, for example, sterile solutions and sterile powders for reconstitution, and the like, that are suitably formulated for injection, liquid and solid dosage forms including, for example tablets, including enteric coated tablets, caplets, gelcaps, capsules, ingestible tablets, buccal tablets, troches, elixirs, suspensions, syrups, wafers, resuspendable powders, liquids and solutions.

On this basis, the pharmaceutical compositions could include an active compound or substance, such as a compound described herein, in association with one or more pharmaceutically acceptable vehicles or diluents, and/or contained in buffered solutions with a suitable pH and isoosmotic with the physiological fluids. The composition could include a targeting agent for the delivery or transport of the active compound to specified sites within the body, organ, tissue, or cell.

As used herein, the term “diluent” refers to a pharmaceutically acceptable carrier which does not inhibit a physiological activity or property of an active compound to be administered and does not irritate the subject and does not abrogate the biological activity and properties of the administered compound. Diluents include any and all solvents, dispersion media, coatings, surfactants, antioxidants, preservative salts, preservatives, binders, excipients, disintegration agents, lubricants, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

Accordingly, in an embodiment, the DHODH inhibitor is administered or used as a pharmaceutical composition comprising the DHODH inhibitor and a pharmaceutically acceptable carrier or diluent.

In an embodiment, the DHODH inhibitor or pharmaceutical composition is administered as a combination therapy.

In an embodiment, the combination therapy includes craniospinal irradiation and/or chemotherapy. The term “co-administration” or “combination therapy” means that at least two compounds or compositions are administered to the subject concurrently, such that effective amounts or concentrations of each of the two or more compounds may be found in the subject at a given point in time. Although compounds according to the present disclosure may be co-administered to a subject at the same time, the term embraces both administration of two or more agents at the same time or at different times, provided that effective concentrations of all co-administered compounds or compositions are found in the subject at a given time. “Co-administration” or “combination therapy” also embraces the use or administration of two or more agents in a single administration or a series of administrations, and/or in the same dosage form or separate dosage forms.

In another embodiment, the DHODH inhibitor or pharmaceutical composition is for administration or use in combination with surgery.

As used herein, the phrase “effective amount” or “therapeutically effective amount” means an amount effective, at dosages and frequencies, and for periods of time necessary to achieve the desired result. For example in the context of treating cancer, an effective amount is an amount that for example induces remission, reduces tumor burden, and/or prevents tumor spread or growth of cancer cells compared to the response obtained without administration of the compound. Effective amounts may vary according to factors such as the disease state, age, sex and weight of the animal. The amount of a given compound that will correspond to such an amount will vary depending upon various factors, such as the given drug or compound, the pharmaceutical formulation, the route of administration, the type of disease or disorder, the identity of the subject or host being treated, and the like, but can nevertheless be routinely determined by one skilled in the art.

The pharmaceutical compositions, formulations, dosages, etc. described herein can be administered for example, by parenteral, intravenous, intracerebral, intraventricular, subcutaneous, intramuscular, intraorbital, ophthalmic, intraocular, intravitreal, intracameral, subtenon, subconjunctival, intraperitoneal, inhalation or spray (e.g. via aerosol), rectal or oral administration in dosage unit formulations containing conventional non-toxic pharmaceutically acceptable carriers, adjuvants and vehicles. The pharmaceutical compositions etc. described herein can be administered as a bolus dose, or as a continuous infusion.

The term “parenteral” as used herein includes percutaneous, subcutaneous, intravascular (e.g., intravenous), intramuscular, or intrathecal injection or infusion techniques and the like. In addition, there is provided a pharmaceutical formulation comprising an active compound, derivative, analog, pharmaceutically acceptable salt thereof or a mixture of any of the foregoing and a pharmaceutically acceptable carrier. One or more molecules of the disclosure can be present in association with one or more non-toxic pharmaceutically acceptable carriers and/or diluents and/or adjuvants, and if desired other active ingredients. The pharmaceutical compositions of the disclosure can be in a form suitable for oral use, for example, as tablets, troches, lozenges, aqueous or oily suspensions, dispersible powders or granules, emulsion, hard or soft capsules, or syrups or elixirs.

Compositions or dosage forms intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present. Pharmaceutical compositions of the disclosure can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811, herein incorporated by reference in its entirety.

Oral or parenteral compositions may be formulated in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals.

The preceding section is provided by way of example only and is not intended to be limiting on the scope of the present disclosure and appended claims. Additional objects and advantages associated with the compositions and methods of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional advantages, objects and embodiments are expressly included within the scope of the present disclosure. The publications and other materials used herein to illuminate the background of the disclosure, and in particular cases, to provide additional details respecting the practice, are incorporated by reference, and for convenience are listed in the appended reference section.

### EXAMPLES

### Example 1: Integrated Functional Genomics and Metabolomic Analyses Identify Targetable Metabolic Vulnerabilities in MYC-Amplified Group 3 Medulloblastoma

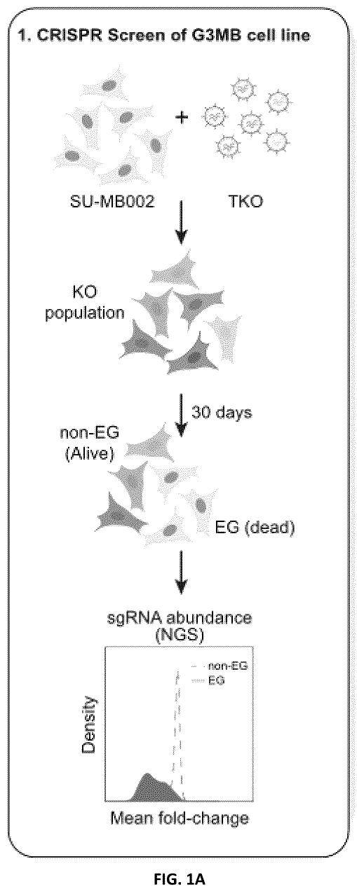

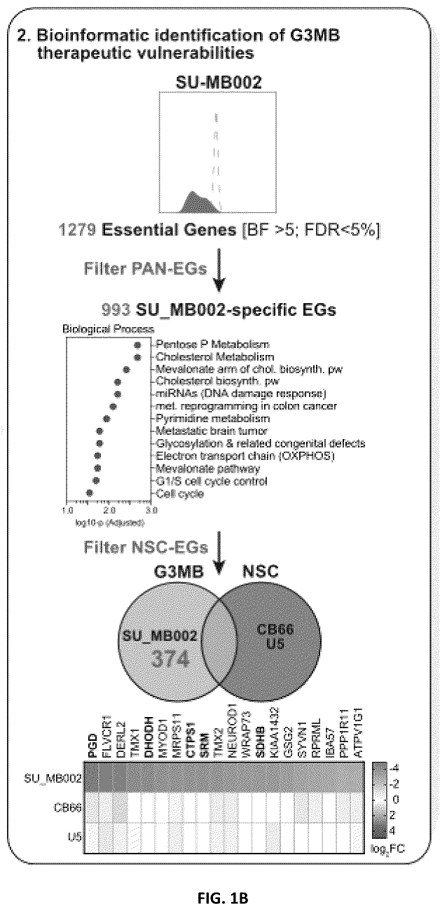

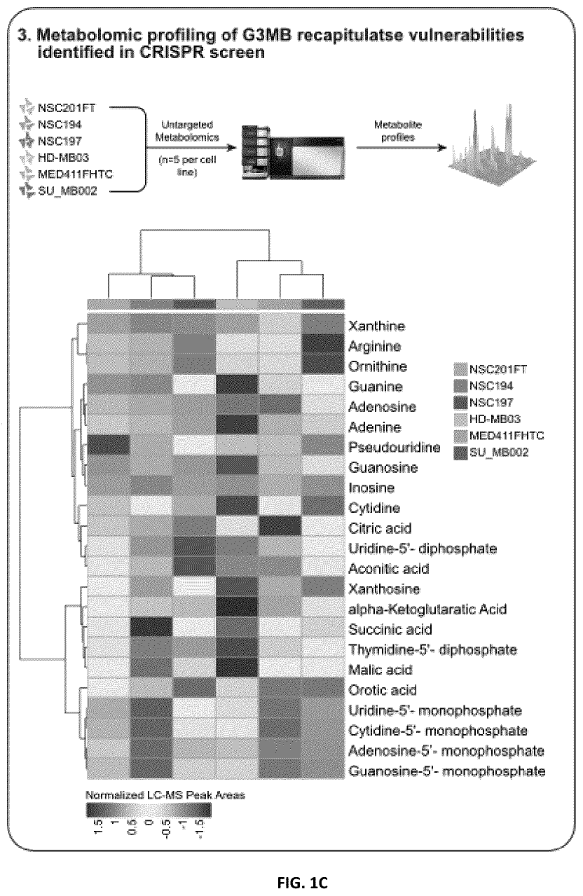

To identify druggable vulnerabilities in G3MB-MYC in an unbiased fashion, a genome-wide CRISPR-Cas9 loss-of-function screen^{19,20} was performed using the patient-derived MB cell line SU_MB002. Briefly, tumor cells were transduced with the TKOv3 library21 at a low multiplicity of infection, and then cultured for 30 days in stem cell-enriching media^{10,17} EGs (Essential Genes) were ranked via next-generation sequencing (NGS) analysis with the BAGEL algorithm21 (FIG. 1A). Interrogation of biological programs (WikiPathways) associated with leading-edge SU_MB002 EGs converged on numerous genes involved in metabolic pathways including the pentose phosphate pathway, OXPHOS, pyrimidine biosynthesis and mevalonate metabolism (FIG. 1B). This suggests that the G3MB-MYC functional genome reflects significant metabolic rewiring, including pathways that we have previously implicated in MB recurrence¹¹.

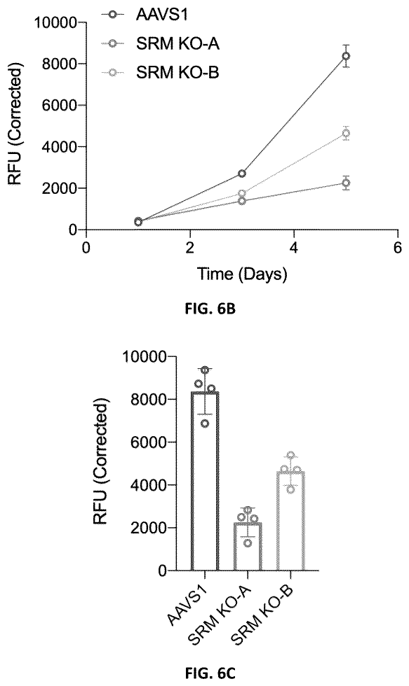

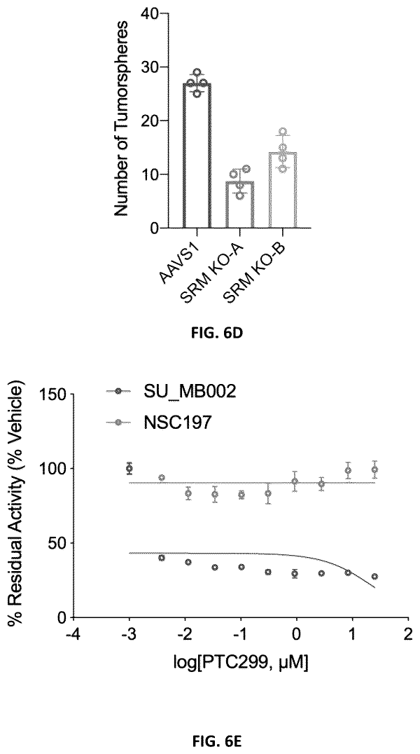

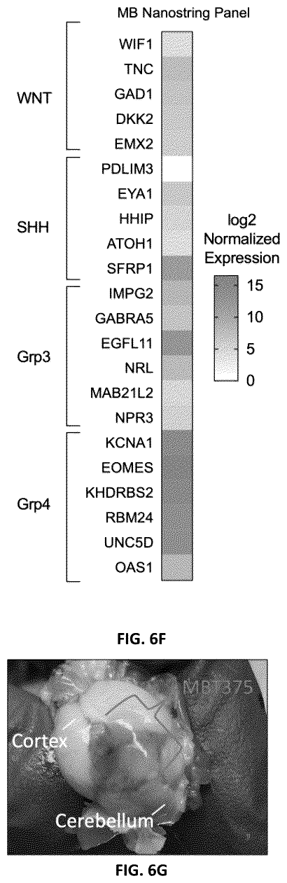

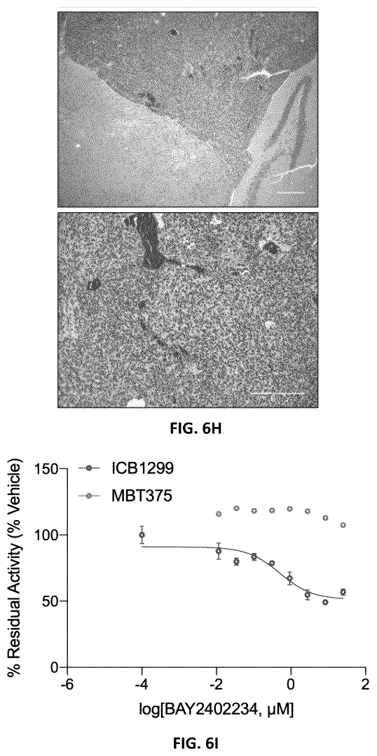

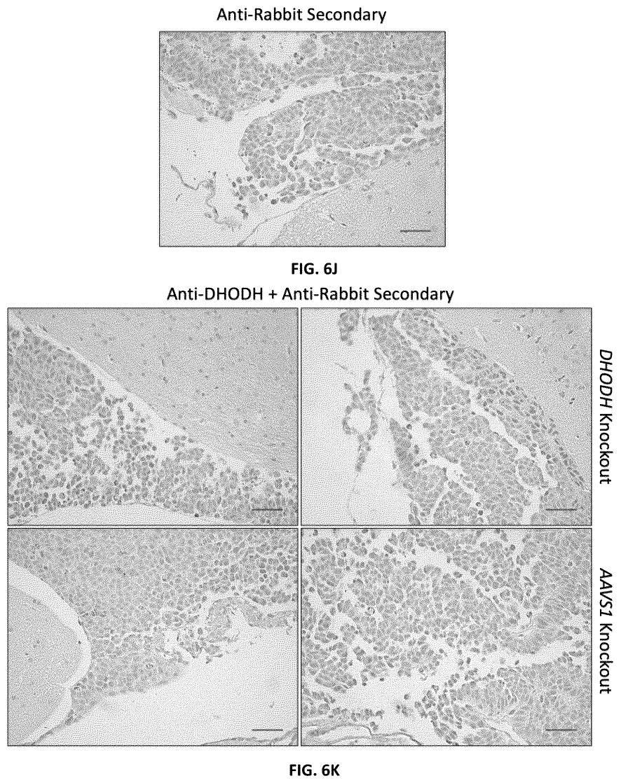

To pinpoint putative cancer-selective therapeutic targets, genetic screening data was integrated with those mined from a study that performed similar screens using two different human NSC lines that were derived from fetal tissues (CB66 and U5) and grown under the same media conditions used in our screen¹⁸. CRONOS dependency scores (DepMap; https:/www./depmap.org/) of the top-ranking G3MB-MYC context EGs were assessed (FIG. 1B) and it was found that indeed nearly half of these genes (8 out of the top 20 ranked EGs) were more essential (defined by negative Chronos score; 22Q2 Public+Score) in G3MB (n=4) compared with all other brain tumors (n=74) (FIG. 6A). Intriguingly, many of the top G3MB-MYC context EGs (PGD, DHODH, CTP Synthase 1 (CTPS1), SRM, and succinate dehydrogenase complex subunit B (SDHB)), have functional metabolic annotations and are amenable to targeting with small molecules. To better understand the heterogeneity of metabolic rewiring in G3MB-MYC, and to select putative therapeutic targets that are broadly applicable across the biological spectrum of tumor and normal specimens, MS-based untargeted metabolomics was performed using three patient-derived G3MB-MYC BTIC-enriched tumor samples (HD-MB03, SU_MB002, and MED411FHTC) and three in-house, donor-derived NSC samples (NSC194, NSC197, NSC201FT) (FIG. 1C).

Hierarchal clustering methods grouped the biological replicates together, demonstrating how NSC and G3MB-MYC tumor cells harbor distinct metabolomic profiles (FIG. 1A). Significant increases in pyrimidine and purine biosynthetic intermediates were detected across all three G3MB-MYC specimens in comparison to the NSCs such as orotic acid (log 2FC=3.2), and uridine (log 2FC=2.9), adenine (log 2FC=5.1), guanine (log 2FC=3.6) and cytidine monophosphates (log 2FC=1.7). Such findings were consistent with PGD, DHODH, and CTPS1 existing among the top G3MB-MYC context EGs in SU_MB002, as each of these genes encode proteins that catalyze different steps in the de novo biosynthesis of nitrogenous bases. Notably, purine and pyrimidine salvage/degradation intermediates were significantly depleted in tumor cells relative to NSCs, suggesting that G3MB-MYC tumor cells sustain their need for purine and pyrimidine metabolites by relying on de novo biosynthesis rather than the salvage pathway. A significant reduction in the levels of arginine (log 2FC=−2.1) and ornithine (log 2FC=−2.2) were also detected in G3MB-MYC as compared to NSCs. This metabolic state has previously been associated with increased expression and enzymatic activity of SRM in polyamine metabolism²². Taken together, these data demonstrate how the unique functional genomic landscape of G3MB-MYC is reflected by concomitant changes in reprogrammed metabolic pathways, and how the integration of these methods identifies prospective, cancer-selective therapeutic targets.

### Example 2: Attenuation of Key Metabolic Pathways Selectively Targets BTICs in MYC-Amplified Group 3 Medulloblastoma

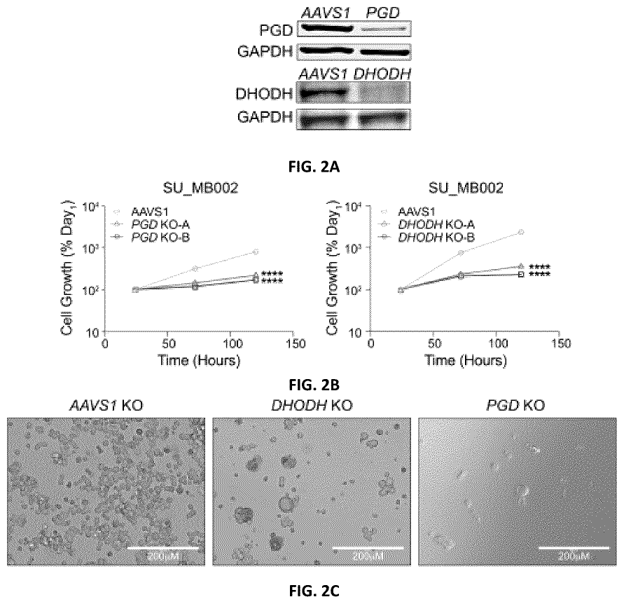

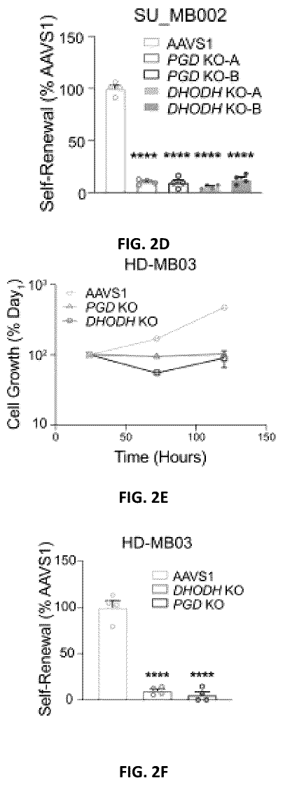

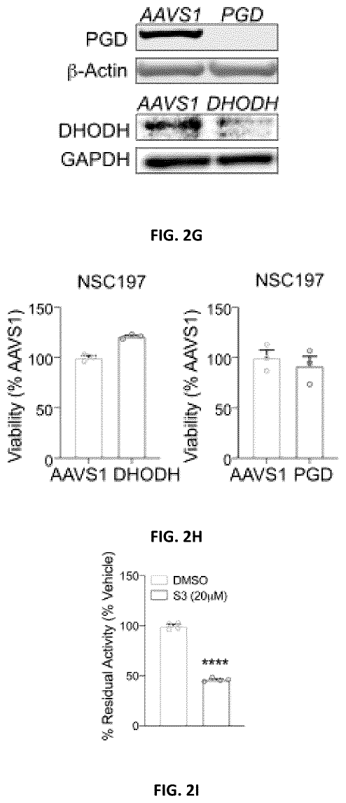

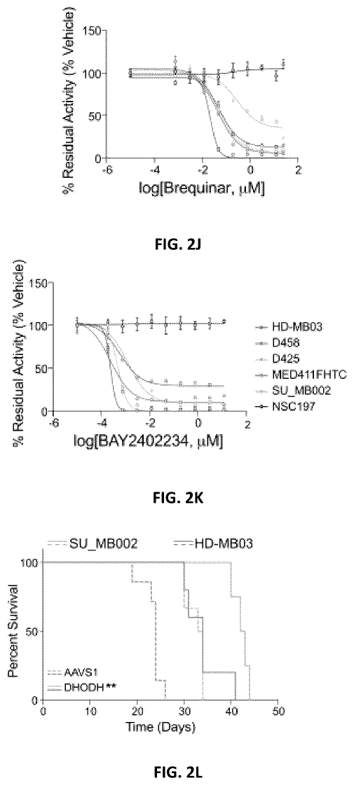

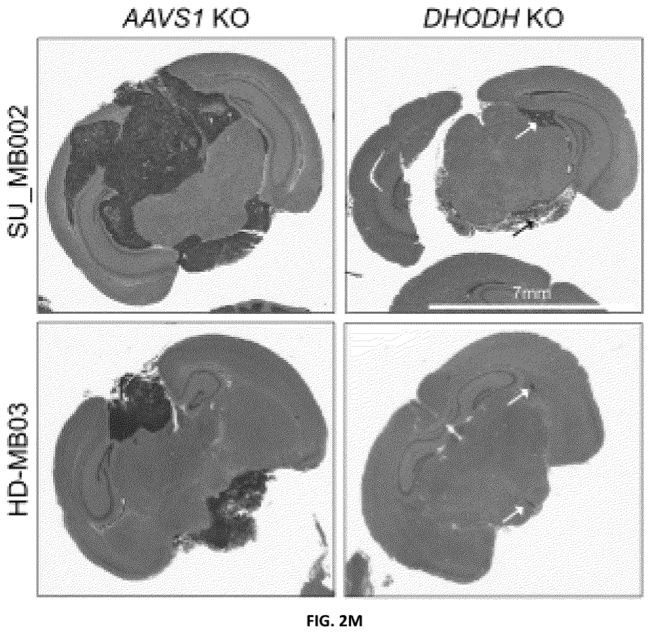

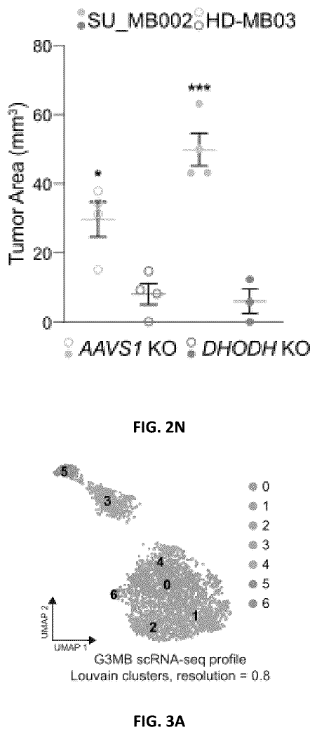

The unique functional genome and metabolome of G3MB-MYC suggests that BTIC fitness necessitates certain metabolic requirements. This hypothesis was explored by knocking out each of three of the highest-ranking essential genes (EGs) with corresponding metabolomic alterations: PGD, DHODH, and SRM in SU_MB002 tumor cells. Knockouts (KOs) were validated using Western blotting, demonstrating a 75-81% KO efficiency for each of four unique sgRNAs tested in comparison to control cell lines that were established by KO of the safe-harbor locus (AAVS1). (FIG. 2A). Loss of DHODH, PGD, (FIG. 2B-C) or SRM (FIG. 6B-C) significantly impaired tumor cell proliferation and viability in comparison to AAVS1 KO, with DHODH and PGD KOs exhibiting stronger effect sizes. Notably, KO of any of these three EGs abrogated their capacity to form tumor spheres (FIG. 2D, FIG. 6D), a surrogate in vitro measure of BTIC frequency^{9,10}. To ensure that these genes are broadly essential to G3MB-MYC tumors rather than being idiosyncratic of SU_MB002, PGD and DHODH were knocked out in a different patient-derived G3MB-MYC tumor cell line, HD-MB0323, wherein the same phenotype was observed (FIG. 2E-F). The non-essentiality of DHODH and PGD in NSC was further validated, demonstrating that loss of either gene does not impair NSC197 cell viability (FIG. 2G-H). These data corroborate the results of the CRISPR screen and highlight DHODH, PGD, and SRM as potential therapeutic targets in MB.

PGD and DHODH are both directly targetable with selective, small molecule inhibitors, but the binding affinities of DHODH inhibitors are much stronger^{24,25}. Indeed, PGD inhibitor S3 hindered the viability of SU_MB002 tumor cells with a half-maximal inhibitory of 20 μM, which is consistent with its binding affinity for PGD24 (FIG. 2I). Hence, whereas PGD is a potential therapeutic target, further medicinal chemistry and the development of more potent inhibitors is necessary. In contrast, DHODH inhibitor BAY2402234 (BAY) reduced SU_MB002 viability with IC50 values ranging between 0.1-1 nM (FIG. 2K, Table 3). Structurally distinct DHODH inhibitors, Brequinar (BQR) and PTC299, similarly affected tumor cell viability with potencies in the nanomolar range (FIG. 2J, FIG. 6E). The in vitro efficacy of BAY against five different G3MB-MYC cell lines [commercial (D425, D458) and patient-derived (HD-MB03, SU_MB002, MED411FHTC)] was further assessed and this metabolic vulnerability was determined to be broadly applicable to G3MB-MYC (FIG. 2K). Importantly, none of these DHODH inhibitors impacted the viability of NSC197 at even the highest concentrations tested (>10 μM). Moreover, DHODH inhibition did not significantly impair the viability of patient-derived group 4 MB cells [slight effect at higher concentrations in ICB129926 and no effect in MBT375 (in-house, group 4 MB PDOX) (FIG. 6F-I)], which suggested that DHODH dependency may in part differentiate MB subgroups. Taken together, these data provide genetic and pharmacological evidence highlighting DHODH as a compelling therapeutic target in G3MB-MYC.

The loss of tumor cell self-renewal in vitro¹⁰ after DHODH KO suggested that this gene is an essential regulator of stemness. To determine whether this change renders a bona fide reduction in BTIC frequency, the gold standard in vivo assay was used⁹. Equal numbers of DHODH or AAVS1 KO (pooled) tumor cells were orthotopically engrafted into the brains of immunocompromised mice. In both SU_MB002 and HD-MB03, DHODH KO significantly delayed engraftment and extended the overall survival of mice by ˜33% in comparison to AAVS1 control mice (FIG. 2L; p<0.01). Histological examination of time-matched PDOX tissues from each cohort of mice further revealed that the DHODH KO significantly reduced intracranial tumor burden (FIG. 2M-N; p<0.01). All mice eventually succumbed to disease. IHC staining of xenografts revealed uniform DHODH expression in PDOX arising from both AAVS1 and DHODH KO cells, indicating that tumor growth in the DHODH KO condition was likely initiated by the 15-20% of untargeted tumor cells that retained wildtype DHODH protein expression in a pooled KO setting (FIG. 6J-K). DHODH is thus necessary for G3MB-MYC BTIC activity in vivo.

### Example 3: De Novo Pyrimidine Metabolism is Enriched in MYC-Amplified Medulloblastoma Subgroups

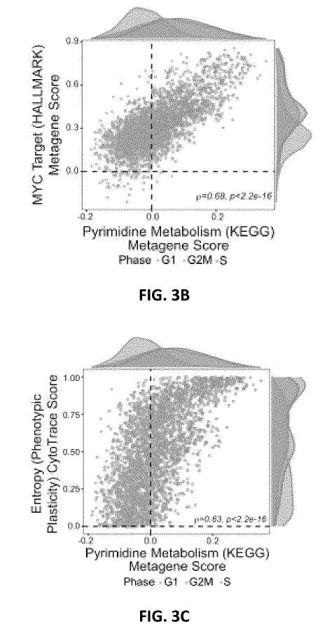

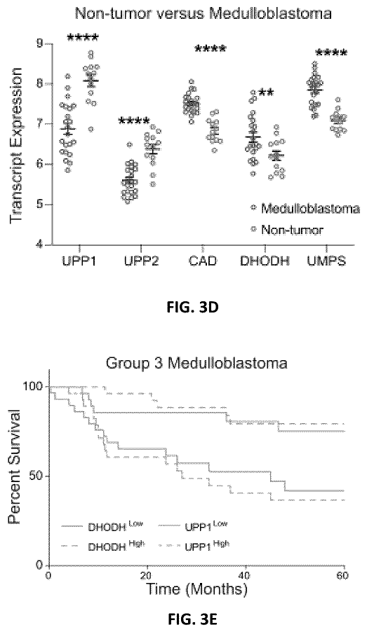

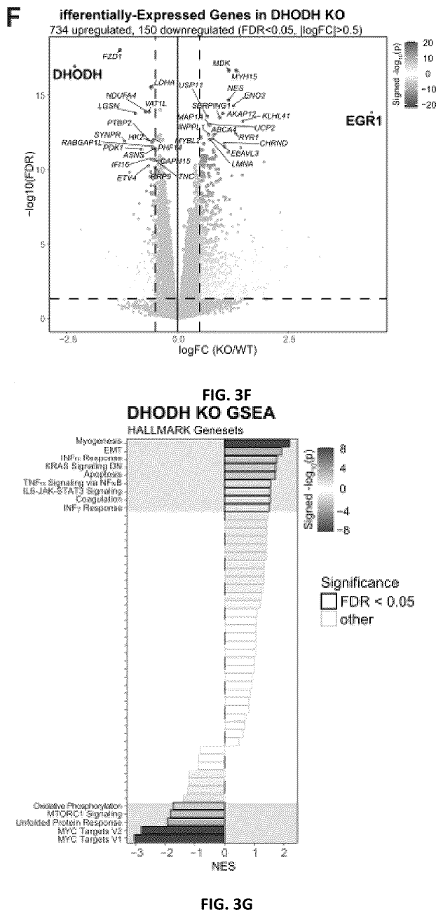

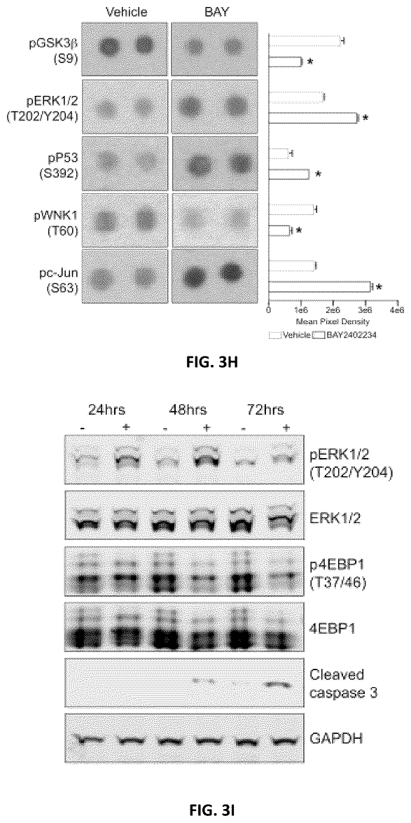

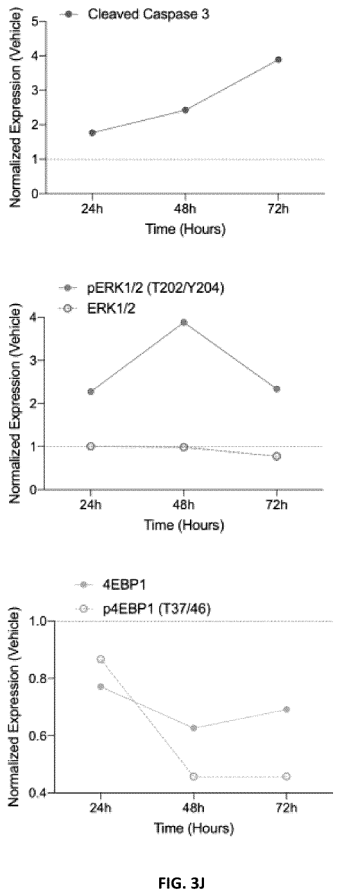

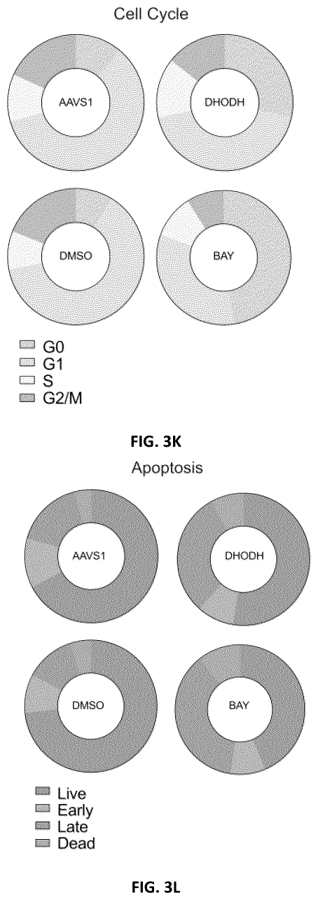

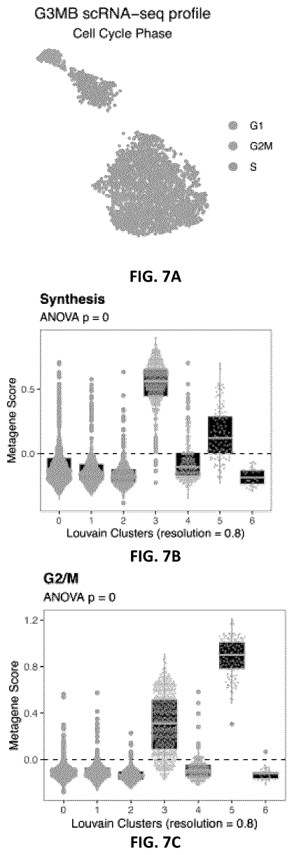

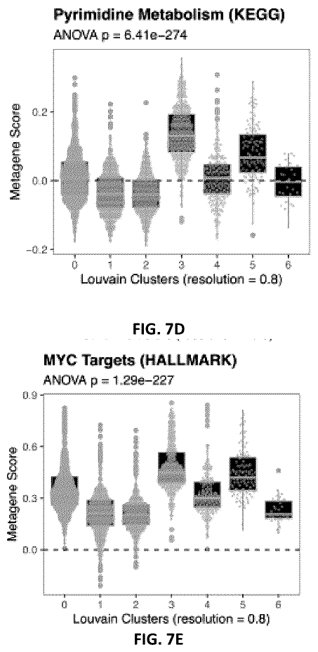

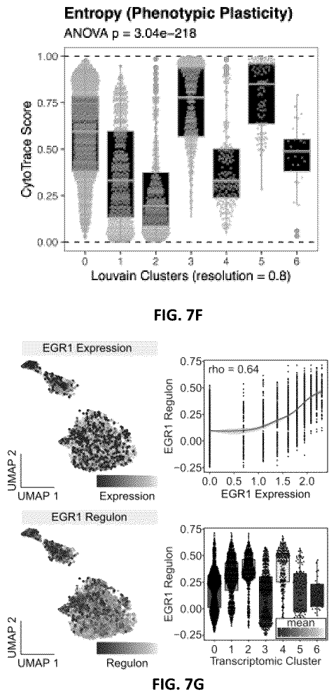

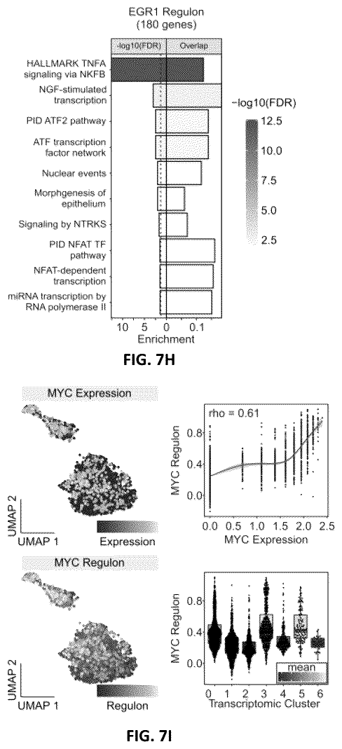

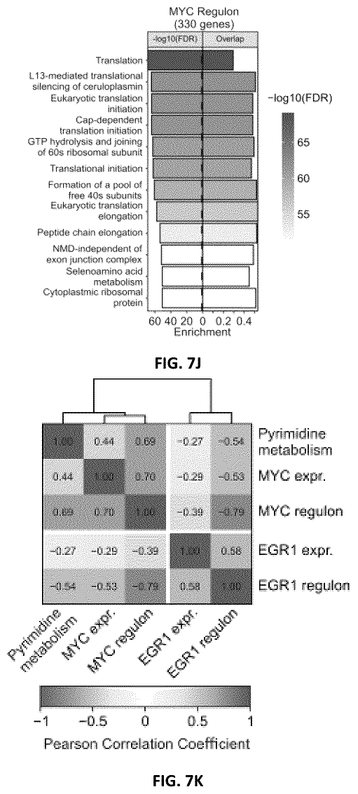

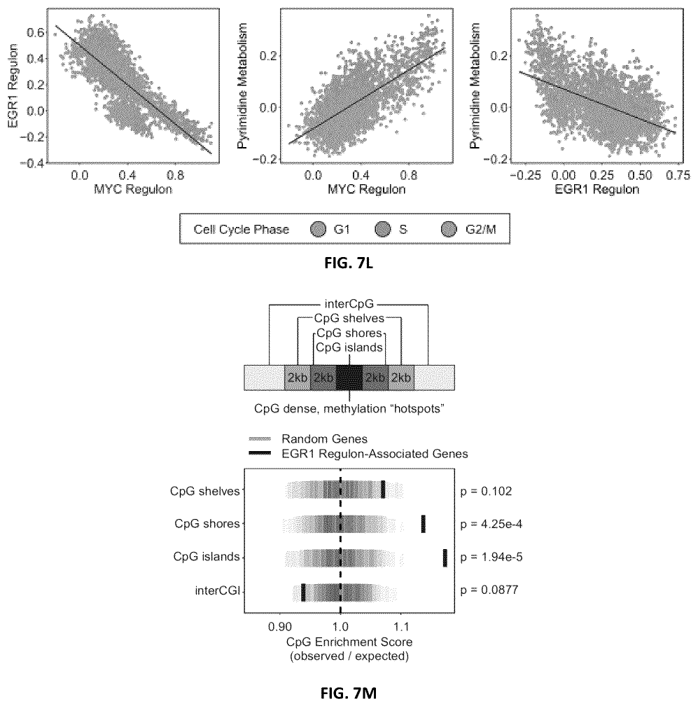

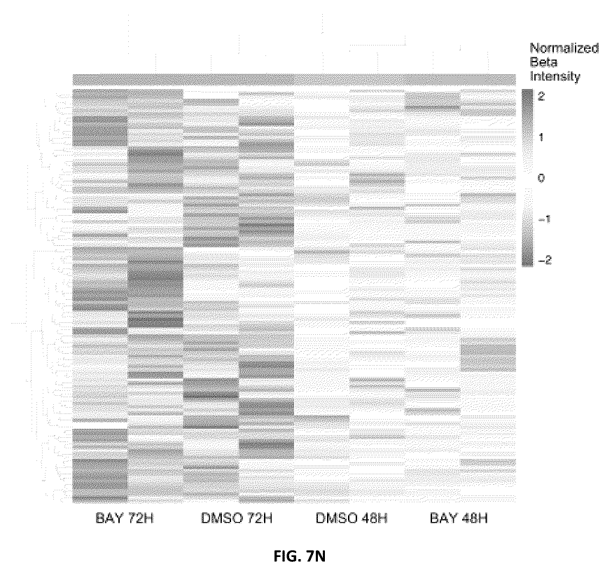

Brain tumor cells often produce heterogeneous, spectrum-like hierarchies of biological programs in situ²⁷⁻³⁰. Publicly available single-cell RNA sequencing (scRNA-seq) profiles of surgically excised G3MB tumors were accessed to learn whether a proliferative tumor cell phenotype enriched in pyrimidine metabolism exists as a distinct cell population in vivo³¹. Analysis of the integrated dataset [6,327 tumor cells from seven tumors] revealed a gradient of gene expression programs associated with proliferation, c-Myc activity, and pyrimidine metabolism (Fig. S2). A distinct population of cells (clusters 3 and 5; FIG. 3A) were identified within the biological spectrum that enriches in proliferative signatures [FIG. 7A-C; DNA synthesis(S), G to mitosis phase transition (G2M)], c-Myc target genes (FIG. 7D), de novo pyrimidine biosynthesis (FIG. 7E), and developmental potential (FIG. 7F)]. Metagene scores of c-Myc target genes and pyrimidine biosynthesis were correlated, with distinct separation by phases of the cell cycle (FIG. 3B). The spectrum of phenotypic plasticity³², a developmental BTIC hierarchy observed in other brain tumors, including GBM²⁹, diffuse intrinsic pontine glioma³⁰ or oligodendroglioma²⁸ was considered to determine where these cells reside. Pyrimidine gene expression scores were found to be positively correlated with Cytotrace score, a numeric measure of cellular phenotypic plasticity³² (FIG. 3C). Together, these data analyses suggest that clusters enriching in pyrimidine gene expression have a c-Myc-amplified proliferative phenotype with high developmental potential.

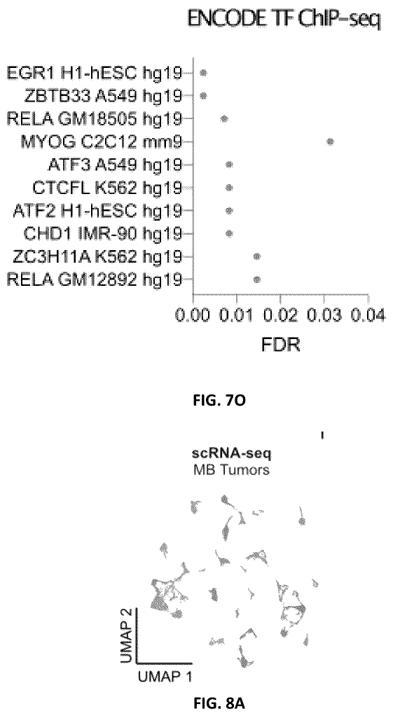

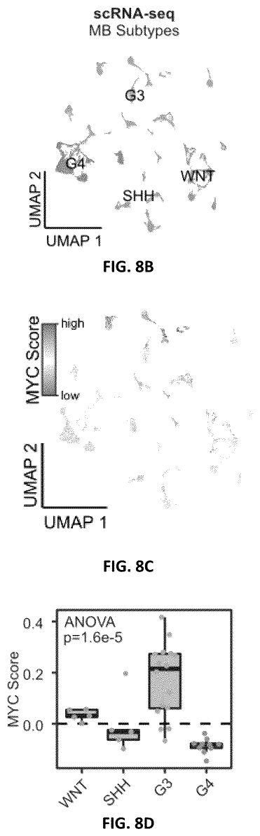

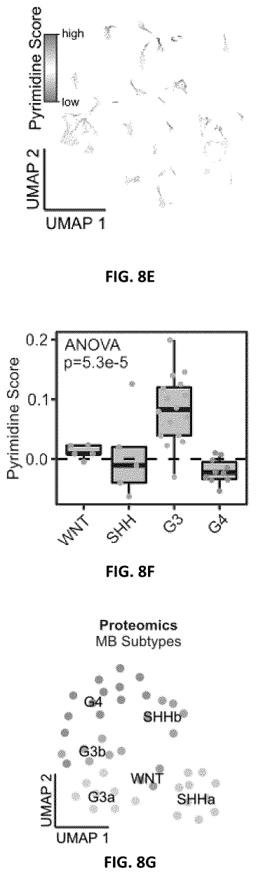

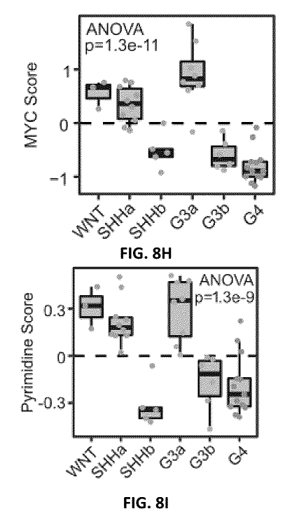

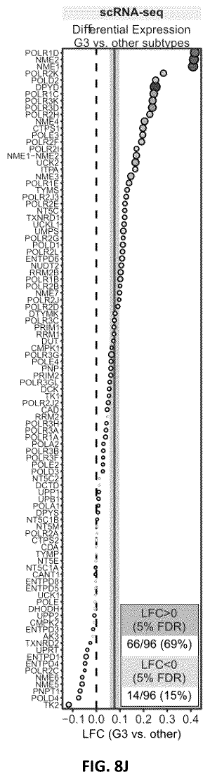

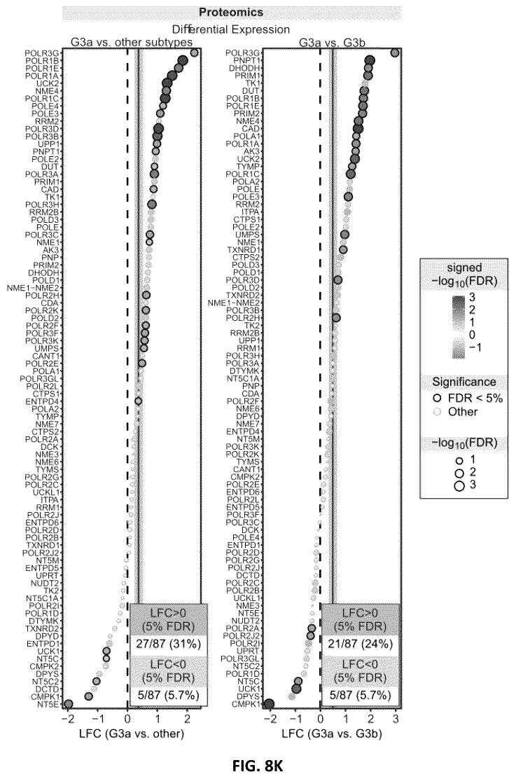

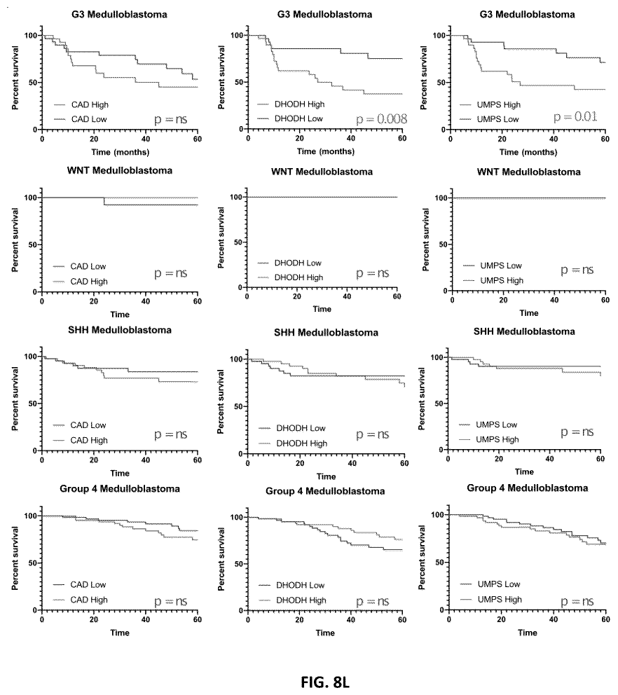

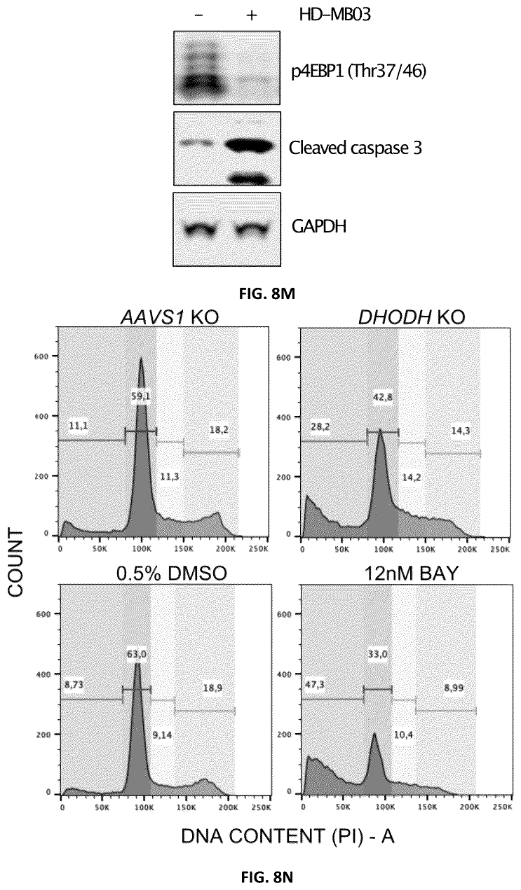

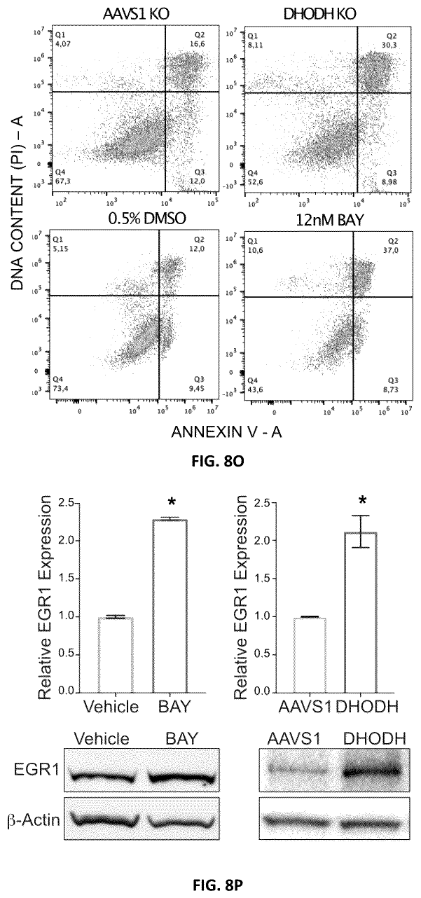

Given the association between c-Myc and the pyrimidine signature, scRNA-seq profiles from all MB subtypes was used to evaluate whether pyrimidine biosynthesis pathway activity is MB subtype-dependent (FIG. 8A-B). In general, G3 tumors had significantly higher c-Myc (FIG. 8C-D) and pyrimidine metabolism (FIG. 8E-F) activity than other MB subtypes. This finding was validated in a complementary proteomic dataset in which G3MB tumors were further stratified into G3a and G3b subtypes (FIG. 8G), with G3a representing a MYC-activated state and G3b representing a G3/G4 intermediary state³³. Proteomic-based MYC and pyrimidine metabolism levels were upregulated in G3a tumors, and at an intermediate level between G3a and G4 states in G3b tumors (FIG. 8H-I). Further examination of pyrimidine metabolism genes in the scRNA-seq data showed that 66/96 genes (68.8%) were significantly upregulated in G3 tumors compared to other MB subtypes, whereas only 14/96 genes (14.6%) were significantly downregulated (FIG. 8J). Proteomic analyses yielded similar results for G3a tumors (FIG. 8K, left). Direct comparison of G3a (high c-Myc activity and poor prognosis) and G3b (low c-Myc activity and excellent prognosis) tumors further highlighted that DHODH was among the top differential pyrimidine metabolism-associated protein between the two states (FIG. 8K, right). Together these analyses support that de novo pyrimidine biosynthesis is associated with MYC-amplified MB subtypes.

Additional analyses of published microarray data comparing primary MB tumors and non-tumor tissue^{34,35} revealed significant tumor-specific upregulation of DHODH and other genes involved in de novo pyrimidine biosynthesis, including carbamoyl-phosphate synthetase 2 (CAD) and uridine monophosphate synthase (UMPS) (FIG. 3D). By contrast, uridine-catabolizing enzymes uridine phosphorylase 1 (UPP1) and 2 (UPP2), were significantly downregulated in MB. Interestingly, whereas high expression of DHODH or UMPS predicted worse overall survival in G3MB patients, high expression of UPP1 was associated with a good prognosis (FIG. 3E; FIG. 8L). None of these genes are prognostic in WNT, SHH or Group 4 MB (FIG. 8L), further corroborating the relevancy of this pathway in G3MB. These data suggest that metabolic reprogramming in G3MB maximizes de novo pyrimidine biosynthesis and represents a potent tumor cell population involved in replenishing tumor mass, wherein c-Myc is a key molecular driver.

### Example 4: DHODH Sustains the Transcriptional Activity of c-Myc and Drives Cell Cycle Progression in G3MB-MYC

To unravel mechanisms of growth arrest in DHODH-deficient G3MB-MYC, the transcriptome of SU_MB002 DHODH KO tumor cells was examined via RNA sequencing (Table 5). Expectedly, DHODH was the most significantly depleted transcript (log 2FC=−2.32) in DHODH KO cells in comparison to AAVS1 KO cells (FIG. 3F). Gene-set enrichment analysis (GSEA) 36 of HALLMARK gene sets identified several biological programs associated with loss of DHODH (FIG. 3G; Table 5). MYC-target genes V1 and V2, and G2M checkpoint genes, were among the most depleted pathways, implying a relationship between pyrimidine biosynthesis and the highly plastic, c-Myc-amplified phenotype that was identified in the scRNA-seq analyses (clusters 3 and 5; FIG. 3A). Hallmark OXPHOS and mTORC1 signaling, which have previously been implicated in G3MB-MYC^{11,37}, were also significantly depleted (FIG. 3G). Hence, DHODH inhibition interferes with transcriptomic and metabolic programs that pertain to G3MB-MYC progression.

In the screen the following genes were upregulated and were sorted by HALLMARK pathway as shown in Table 5: MDK, MYH15, NES, ENO3, EGR1, AKAP12, USP11, SERPING1, KLHL41, MAP1A, UCP2, RYR1, INPPL1, ABCA4, MYBL2, ELAVL3, LMNA, CHRND, PITPNM1, MYOG, TP53I11, MYL6B, SPARC, MYO6, C11orf96, PCDH9, NPTX2, ITGAV, IDH2, E2F2, MLLT11, NDRG4, ASF1B, ANTXR1, GABRQ, IKZF1, MKNK2, TUBA4A, LTBP4, SYT11, ERBB3, VIM, CCL26, CELF3, SLC17A7, BTG2, TSPYL2, HSPB1, CHRNA1, ZNF385A, MOV10, PLPPR2, RTN4RL2, LOXL1, PGAM2, BLCAP, TSPAN11, TRIL, BRPF3, SYNJ2, SMPD3, EEF1A2, SALL2, CFAP57, MRC2, AANAT, PLCXD1, PTPRD, KREMEN2, RUNDC3A, CCDC88B, ZSWIM8, RAB6B, ARL4C, FAM212B, SPTBN4, LIMCH1, TIMP2, C1orf106, EMP2, SMAD9, SPC24, APOBEC2, TNNT2, ANKLE1, PALLD, AHNAK, ABAT, RAB26, TSC22D3, STC1, BCL9L, CRTC1, MAP3K12, TMSB15A, CPT1C, THBS3, ARHGEF40, C9orf72, SRGAP3, MYBPHL, KANK2, GRIK5, EPHB2, TNNT1, ANXA2, FRMD3, ADAMTS4, NEB, TM7SF2, CMTM3, TMEM2, TMEM38A, FOS, PDLIM3, ZNF641, HIST2H2BE, RAB3B, MKX, SLITRK5, DDR1, DYRK1B, CRMP1, NRXN2, EPHX1, KAT2B, E2F8, FN3K, PLEKHH3, ITGA4, ZNF503, ARMCX2, S100A4, CCHCR1, CCDC150, HSPB8, H2AFJ, NUMBL, PRPH, PPP4R4, SYN1, EMP3, TRIB2, CACNA2D4, CDH2, DUSP26, NRG1, CYFIP1, LIMD2, DLC1, SSC4D, WDR62, RHOC, PKN3, CDH15, OPTN, ATP10D, KIF1A, IQGAP3, PPP1R14A, NAV2, CACNA1F, CORO2A, FNDC5, HOMER3, ATP1B2, BAALC, ANTXR2, MXD1, CNGB3, APCDD1, UBAP1L, MDGA1, ANO8, CDH17, CACNB3, PAK3, KCNK2, STAC3, ZNF93, NCKIPSD, SERAC1, SLC4A3, SPSB4, SLC25A27, KIF19, SLC44A5, YPEL1, CACHD1, GRM2, LDB3, KIAA0922, PDLIM4, CASP6, CACNG5, TCEAL7, GPRC5B, TSC22D4, TRIM5, PHLDB3, SIPA1, SYNPO2L, FOXN4, STAT2, PAQR8, GMIP, SNCG, PPP1R17, FCGRT, EYA2, CDKN2D, INSM1, DPF1, UCHL1, KIAA1456, MFAP4, ZNF616, MDFIC, DCN, SPTBN5, CNIH2, PDE4DIP, IL11RA, CLIP3, ALPK2, NPHS1, PLK2, CAPN5, ATL1, KLF12, IL17RB, ITPRIP, PLXDC2, ABCC8, ERFE, COL1A2, SPATA33, ATP6V1G2, PCDHB16, BDH1, TRIM36, RGS2, ADCY7, RP11-6L6.2, TMEM44, SYBU, KAZALD1, FRAS1, AMPH, SPN, OTOF, CPXM1, ANKRD6, FAM101B, KCTD13, SAMD5, HIVEP2, KCNF1, MPDZ, ATP1A2, PGM2L1, SNN, MELTF, CCDC188, SCN3A, PCDHB5, LRTM1, ATXN7L2, FITM1, TTPAL, LAD1, CARD8, SMARCD3, PROX1, DUSP13, CHRNG, ADGRL3, CBX4, MAPK11, BCL6, CAMK2B, CD82, CLTCL1, IFI6, BRICD5, SNAI1, GPR173, GPR37, MYLPF, ARAP3, GPRASP2, HFE2, SLC22A8, MSRA, JADE2, UNC13D, UNC45B, UBE2L6, STK17A, RCAN2, ZYX, MAST3, TMEM130, RNF182, TMTC1, ERAP1, FAM135B, CLEC11A, ARPP21, PLA2G12B, TNNC2, EHBP1L1, KITLG, ZCCHC24, HIF3A, MYBPH, IGSF22, CYP2E1, IFI35, HAS3, OLFML2B, COL4A4, MFSD6, ACVRL1, MYH3, C2CD4C, ZNF385C, ARHGAP29, TIGD7, TMEM55A, RGS4, OLFM2, ZNF677, ABCB4, DOK6, SMTNL1, SLIT2, ADTRP, ZNF726, PCDH19, ZNF703, ZBTB8B, PDGFC, EPHA4, SLC25A34, TUBB4A, RUNX1T1, QRICH2, ARID5B, TP53I3, HHEX, DOCK11, SPACA6, IL17RC, ANO3, TCAP, S100A1, GSDMB, CCDC184, PCDH10, TCN2, GEM, SLC39A12, MYL1, ANGPTL2, SMPD1, PLD1, SCCPDH, BLOC1S3, CHFR, PLAUR, ZNF583, TOMM5, KCNMB4, CNFN, PLCB1, KIAA1462, AFAP1, GRIK2, RADIL, ESYT3, CFAP65, LGI2, FAM122C, ZNF844, CAPN6, MBNL3, UNC80, LTBR, TAGLN3, FSTL1, GPR19, EID2B, CAP2, MAGEH1, COL19A1, FAIM2, KCNJ14, ISM1, ERBB2, PKIB, ARHGAP25, NLRP1, DENND3, CPLX2, SUSD1, PTPRO, OSBPL7, IP6K3, HSF4, KDR, TNS1, SLC2A6, CCDC177, PIK3IP1, NEXN, COLGALT2, NR2F1, ARC, TCF7L1, FUT9, THBS4, TRIM25, SNED1, DHRS1, PLEKHA4, LRRTM2, PCDHGB7, HLX, MYO18B, TCEA2, GPR161, SP9, CLVS2, HTR3A, CORO6, ARHGEF33, MYADML2, MYLK4, ZNF568, F13A1, SLC37A2, HBEGF, C14orf132, THSD7A, GALR1, SSTR1, KLHL40, C1orf226, RDM1, GRIN2D, BGN, NRIP3, TRIM54, CXCL14, ANK1, CEP170B, CARD9, TRERF1, ADGRL1, PTGS1, HAND2, LARP6, SH3RF1, ALPK1, MCHR1, ZNF611, ZNF543, STXBP6, SEMA3D, VASN, PIK3C2B, TMBIM1, ADAM12, ARMCX4, KCNG3, CCDC159, SAMD9, IFITM3, TTLL3, ZNF425, PLAT, TP53INP1, PPP1R16B, FAM196A, OR51E1, ELK3, CLDN1, LTB4R, POLN, MUC20, GGT1, PPP1R13L, CDKN2B, GK, MCOLN3, ABLIM3, NRXN1, NIPAL1, LACTBL1, PI3, ZNF467, MYPN, TMSB4X, RNASEL, SLC48A1, ZNF671, CRX, FLT4, CLDN5, BCHE, ARHGAP36, ABHD8, BAIAP3, ERP27, PCDHGA1, PODN, SYTL1, GLRA1, SFTPD, TIGD1, MYOM3, NDST4, SORCS1, NOTCH1, CCDC102A, SOWAHA, SLC4A10, RELB, TAPBP, TUBB3, PKNOX2, FAM213B, ADGRB1, ZNF385B, TINCR, ARHGAP10, ALDH1A2, TRIM56, MIEF2, SLC12A8, EGLN3, OLFML3, MEGF10, SHD, XIRP2, TCEAL3, CAV3, ADGRB2, ATOH8, SP5, ADAMTS1, DLGAP3, PLXNC1, SHROOM1, GRIA4, RBM24, PYROXD2, MAGEA11, IQCE, ADCY6, AEBP1, CDKN1C, PCDHB2, PYGM, MSR1, ZNF66, CHODL, CIDEA, ARHGAP24, MBLAC1, VANGL2, MAGEB6, MYO5B, APLNR, USP44, FAM105A, AGBL3, AARSD1, LINC00672, HID1, GBP2, GPLD1, ANKRD65, SCN2A, ZNF528, PITPNM3, TCF7, NOX4, GULP1, AHNAK2, PLIN4, BDNF, ZNF221, KCNC2, SYT6, VWCE, SHC2, RET, MXRA5, HDAC9, RBP1, FBN3, WFIKKN1, SEMA6B, SLC22A17, RNF112, CRISPLD2, EPAS1, SLC16A4, SVIL, LMNTD1, GPC1, DEF6, TOX, XIRP1, BNIPL, PPARG, CCDC155, LRRN2, SLC16A2, HRC, DMBT1, MAPK4, SCEL, DACT3, ADAMTS20, TMEM98, NECAB2, TMEM88, DBNDD2, SNCAIP, ANKRD45, CACNB4, SCG5, ZPLD1, SAMD3, HIST1H3G, C16orf62, NFAM1, THNSL2, AIFM3, SH3D21, EMILIN3, HLA-DQB1, PARP12, SLC26A4, TRIM46, PAPSS2, SLC38A3, ZNF718, ZNF682, IL12A, LINC00176, SOX8, MCIDAS, ZNF233, MSS51, RILP, PHLDA3, RORC, DNER, ZNF836, SERINC2, PAGE5, COL25A1, BHLHE41, ZNF141, LZTS1, PECAM1, HIST1H2BF, LMOD3, B3GNT2, CCDC152, CYR61, CACNG4, CRISPLD1, CYP3A5, KCNN2, ZNF853, ZNF418, ZNF554, ZNF223, NAV3, NKX3-2, PLCD4, RHBDF1, TBC1D3L, C15orf65, CKM, CEMIP, SLC27A3, MESP1, MEOX1, SNTB1, PRSS21, CACNG6, TNFRSF11A, AGTR1, KIF7, MOB3B, PIWIL4, SCN5A, JAK3, C1QL4, SLC7A14, RIMS1, B3GNT9, DCLK3, MAGEB1, TMPPE, SLC16A9, PEG3, RP11-192H23.4, UPP1, LMX1A, RP3-382110.7, PTGR1, ATP8A2, CRTAC1, SYT2, THAP8, EVC, TMEM35A, LHX2, NYNRIN, TAS1R1, PTX3, TMEM17, TBX15, ICA1, GCNT2, PGPEP1, and SEC14L2.

In the screen the following genes were downregulated and were sorted by HALLMARK pathway as shown in Table 5: FZD1, DHODH, LDHA, NDUFA4, VAT1L, LGSN, PTBP2, PHF14, SYNPR, HK2, RABGAP1L, ASNS, PDK1, IFI16, TNC, CAPN15, ETV4, RRP9, SPAG1, ANKS1B, NSMCE1, TFB2M, BNIP3, C7orf50, PRTG, GABRR3, NAT8L, GPR155, TAF4B, MPP4, LRIG3, SMIM24, EFEMP1, MRPL24, LNP1, DYNC1I1, PATJ, FER, SPATA18, MAGOHB, ELP4, TMEM45A, RP11-244E17.1, HMCN1, EPB41L4B, MAML3, CEBPB, NEGR1, METRN, LIAS, PIGF, PTGER4, LYPD6B, ACOX2, TFB1M, RTN4IP1, GNB1L, RPL26L1, TMEM119, CTXN3, ANKRD37, PEX12, SLCO1B3, ELFN1, RNF152, METTL12, MAFF, MT1G, C2CD4A, LIN28A, COQ2, ZNF19, CNTNAP5, FNTB, DNAH10, FOSL2, CLYBL, ERMARD, NID1, FABP12, TMEM116, FAM86C1, STAG3, SMN1, GLRA3, LRRC27, CCNJL, IQCG, CCDC113, RPA4, DNAH5, AJUBA, NKPD1, PLPPR5, ANKRD34A, CSMD1, MDH1B, CCDC61, TDRD6, TBC1D19, CSF2RA, PCDH15, TRIM16, LRRIQ1, WNT10B, PPM1J, DCSTAMP, HMGCL, PRICKLE2, NLRP11, HESX1, STK32A, ELOVL3, HEPACAM, EVPL, NME5, SLITRK6, RPP21, FDX2, KISS1R, PAX9, CDH23, CBX7, PIK3CD, BHLHE40, FMOD, HYAL3, ST6GAL2, GPAT3, HLA-DRB1, ZNF497, C19orf57, NCMAP, SH3BGR, ZNF18, SUGCT, MOBP, AC008522.1, LGI1, ETFBKMT, ATP6AP1L, RHAG, CA8, GPC4, EPHB1, FAM53A, TM4SF18, PHOX2B, PHEX, and SLC16A3.

To identify signaling changes accompanying DHODH inhibition in an unbiased fashion, changes in the phosphokinome of SU_MB002 cells were measured after treatment with BAY. Increased phosphorylation of ERK1/2 (T202/Y204; T185/Y187) and c-Jun (S63) (FIG. 3H) were detected in BAY-treated cells in comparison to vehicle-treated cells. Time-course Western blotting confirmed that ERK1/2 becomes phosphorylated within 24 h of BAY treatment, peaks at 48 h, and then decreases in expression at 72 h (FIG. 3I-J). BAY treatment significantly depleted the activity of the mTORC1 pathway in DHODH KO SU_MB002 cells (FIG. 3G). The phosphorylation of eukaryotic translation initiation factor 4E binding protein 1 (4EBP1), a direct and indirect target of mTORC1, decreased in a time-dependent fashion with BAY treatment (FIG. 3I-J, FIG. 8M). These findings corroborated previous work that had implicated ERK1/2 silencing of mTORC1-mediated translation in activating apoptosis in cancer cells under severe metabolic stress³⁸. Indeed, both pharmacological inhibition or DHODH KO in G3MB-MYC induced both Go cell cycle arrest, determined via propidium iodide assessment of DNA content, (FIG. 3K, FIG. 8N) and apoptosis, determined via cleaved caspase 3 expression (FIG. 3I-J, FIG. 8M) and Annexin V staining (FIG. 3L, FIG. 8O). Such findings were also consistent with the enrichment of the hallmark apoptosis gene set in DHODH KO SU_MB002 cells (FIG. 3G). Without wishing to be bound by theory, taken together these data describe a model whereupon loss of DHODH activity, G3MB-MYC tumor cells are unable to fuel the rapidly cycling, MYC-amplified transcriptome. Pyrimidine starvation induces a metabolic stress response, which activates ERK1/2-mediated signaling, leading to suppression of mTORC1 activity, and initiation of apoptosis.

A wide range of enriched biological processes were detected upon genetic loss of DHODH, including myogenic differentiation, apoptosis, DNA damage response and signaling via tumor necrosis factor α (TNFα). This broad activation of biological programs was reflected in the 734 upregulated genes, in contrast to only 150 downregulated genes (FIG. 3G). The most differentially upregulated gene EGR1 (log₂ FC=4.37) encodes transcription factor early growth response 1 (EGR1), whose transcriptional activity has been implicated in almost all pathways overrepresented in DHODH KO cells, including myogenesis³⁹, TNFα signaling via NF-kB, and apoptosis⁴⁰. Prior work has demonstrated that EGR1 is a tumor suppressor induced by JNK/c-Jun and ERK1/2 signaling in response to DNA damage, suppressing tumor growth via TNFα-driven cell cycle arrest and apoptosis⁴¹. EGR1 was upregulated at the protein level via Western blotting in BAY-treated (FIG. 8P; left) and DHODH KO (FIG. 8P; right) tumor cells relative to their respective controls.

The scRNA-seq profiles described above were used to examine whether G3MB patient tumors express functional EGR1 through regulon analysis (FIG. 7). EGR1 expression and regulon activity were negatively correlated with MYC regulon activity, as well as pyrimidine metabolism (FIG. 7G-L), with demonstrable underrepresentation in the previously described scRNA-seq clusters 3 and 5 (FIG. 3A, FIG. 7G). Among the DEGs upregulated in DHODH KO cells (FIG. 3G), EGR1 regulon-associated genes (inferred from scRNA-seq) were significantly enriched [9.4% (17/180) genes; p=3.39e-03, hypergeometric test]. In agreement with changes in c-Jun/ERK1/2 signaling upon DHODH inhibition, functional enrichment of EGR1 regulon-associated genes (inferred from scRNA-seq) implicated TNFα signaling via NF-kB as the top pathway downstream of EGR1 (FDR=2.59e⁻¹⁴, 22/178 gene overlap, hypergeometric test) (FIG. 7H). EGR1 was recently shown to regulate neuronal differentiation and maturation in the postnatal brain⁴², affecting target gene transcription by recognizing CpG dinucleotide-rich promoters. Consistent with such a mechanism, EGR1 regulon genes were significantly overrepresented in genomic regions associated with CpG islands and shores, but not CpG shelves or CGI islands (FIG. 7M).

To directly assess epigenetic changes associated with DHODH inhibition, genomic DNA was isolated from tumor cells treated with BAY or its vehicle for 48 h or 72 h and measured global epigenetic changes (Ilumina EPIC Methylation Array) (FIG. 7N). Interestingly, whereas methylation profiles were similar after 48 h of treatment (only 18 differentially methylated positions (DMPs)), they diverged significantly after 72 h (751 DMPs). Such temporal changes in the epigenome are in keeping with the ERK1/2 temporal signaling kinetics (FIG. 3I-J). The distribution of the DMPs was determined and those located in promoter islands were focused on in keeping with the EGR1 consensus recognition (considering TSS1500, TSS200 bp, 5′UTR and 1st exon as promoter regions). 2/18 and 123/751 DMPs were identified in promoter islands at 48 h and 72 h, respectively. These differentially methylated CpG islands were interrogated using Enrichr⁴³ and these genes were found to show strongest associations with EGR1-, NF-KB-, and MYOG-target gene sets (FIG. 7O). Hence, methylation analysis of tumor cells treated with BAY showed enrichment for the same differentially expressed pathways observed after DHODH KO. Together, these data suggest that transcriptomic and epigenetic changes that occur upon loss of DHODH are associated with EGR1 genomic activity.

### Example 5: DHODH Inhibitors Exert On-Target Therapeutic Effects by Altering the Metabolome and Lipidome of G3MB-MYC in a Uridine-Dependent Manner

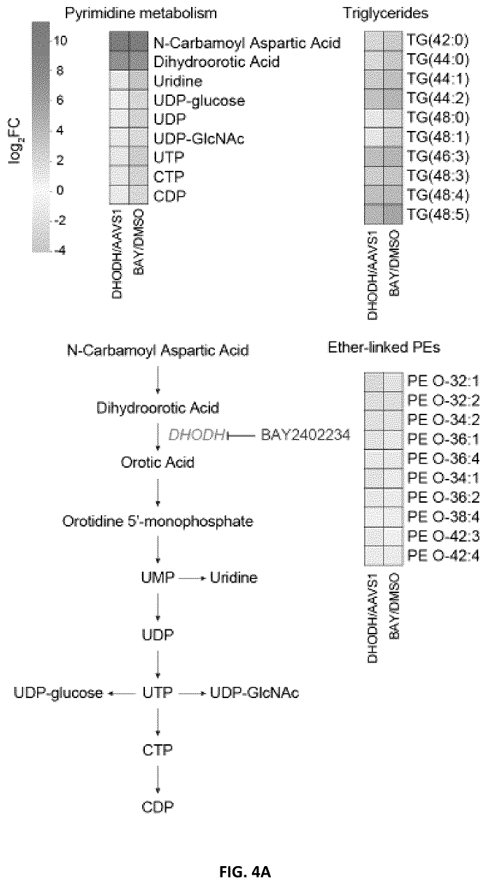

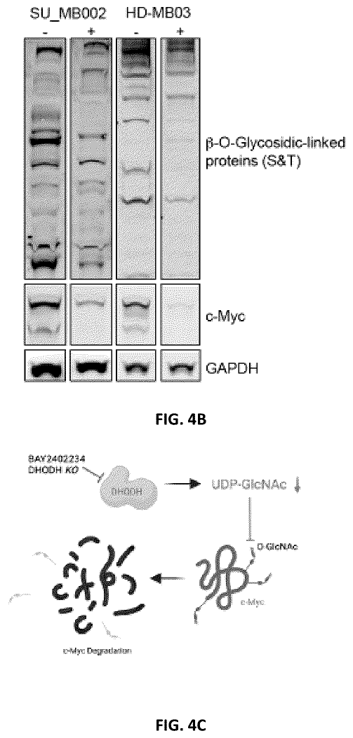

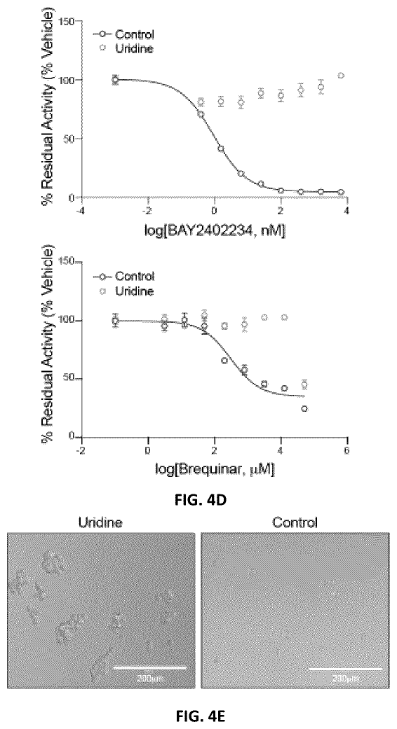

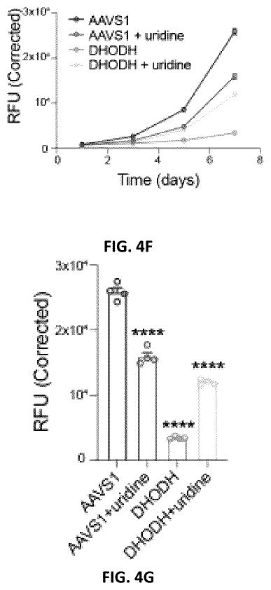

The metabolome and lipidome of SU_MB002 tumor cells after either genetic or pharmacological inhibition of DHODH activity were profiled to understand how the loss of DHODH impacts G3MB-MYC metabolism (FIG. 4A; Table 4A, B). There was a striking concordance between dysregulated metabolites detected via both genetic and small molecule inhibition of DHODH, further highlighting the on-target activity of BAY. DHODH inhibition yielded substantial increases in the abundance of de novo pyrimidine biosynthetic precursors and intermediates (substrates and products of CAD): aspartic acid, carbamoyl aspartic acid, and dihydroorotic acid. Such findings established that tumor cells attempt to reroute metabolic resources into the de novo pyrimidine biosynthesis pathway upon loss of DHODH. However, despite such metabolic feedback, the abundance of uridine and uridine-related metabolites remained significantly depleted.

Uridine metabolite depletion may impact not only the macromolecular demands of the rapidly cycling cellular phenotype identified through scRNA-seq analysis (inducing cell cycle arrest), but also the potentiation of oncogenic signaling programs. For example, UDP-GlcNAc, which is an oncometabolite required for O-linked β-N-acetylglucosamine (O-GlcNAc) mediated stabilization of c-Myc in DHODH-dependent cancers^{44,45}, was significantly reduced by both methods of DHODH inhibition (FIG. 4A). To determine whether reduced UDP-GlcNAc abundance influences O-GlcNAcylation of proteins and/or expression of c-Myc at the protein level, the abundance of β-O-glycosidic-linked proteins (Ser and Thr residues) and the expression of c-Myc protein in HD-MB03 and SU_MB002 tumor cells treated with BAY or its vehicle, were measured using Western blotting. BAY treatment resulted in a global reduction in the abundance of O-GlcNAc-conjugated proteins, and a significant reduction in c-Myc protein expression relative to controls (FIG. 4B-C). Hence, the reduced c-Myc transcriptomic activity observed after DHODH KO occurred in part via known metabolite-protein interactions.

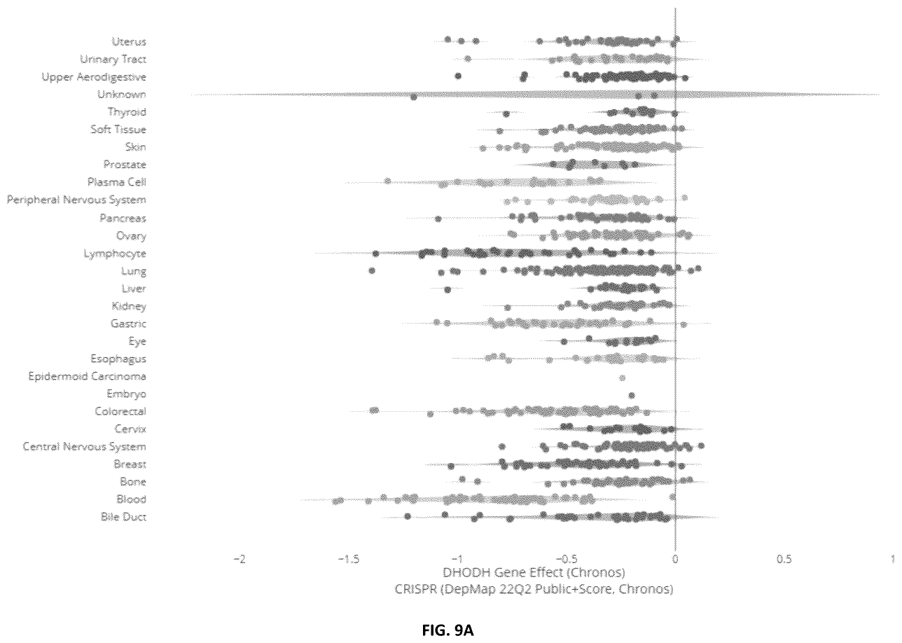

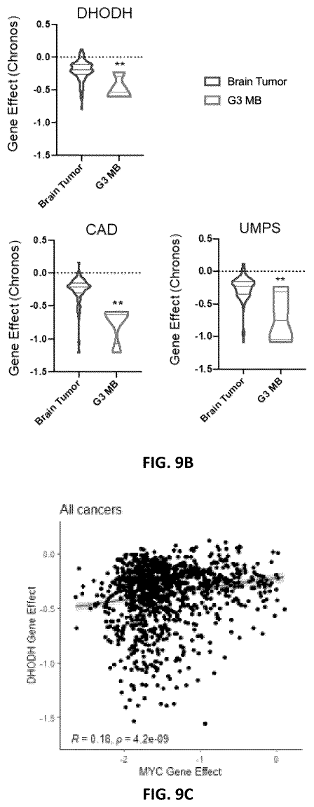

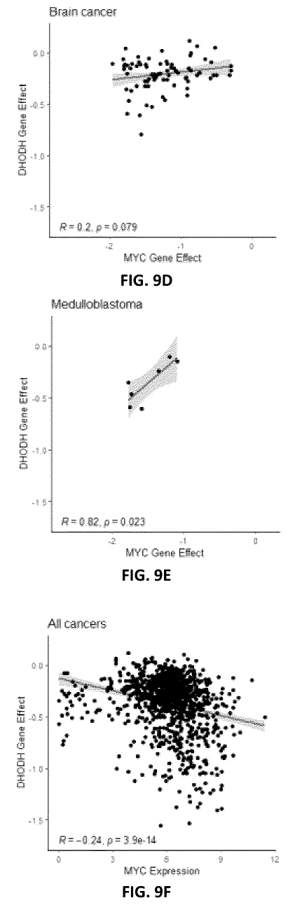

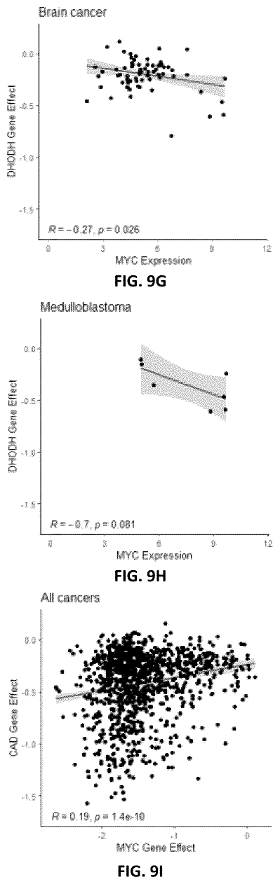

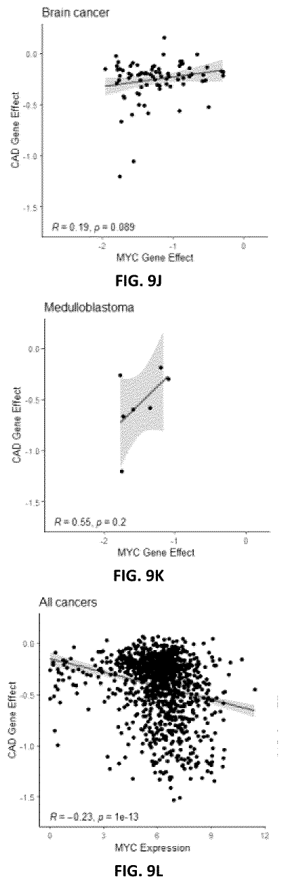

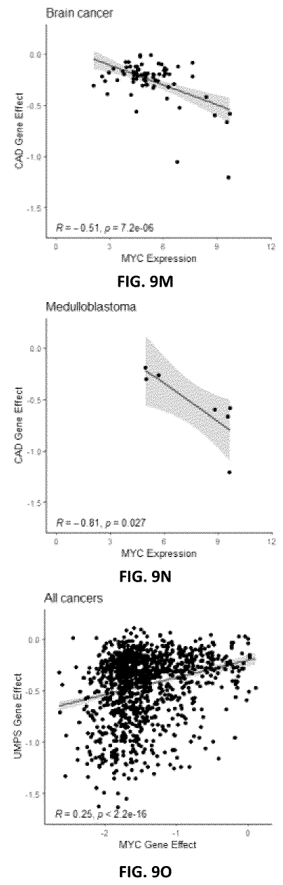

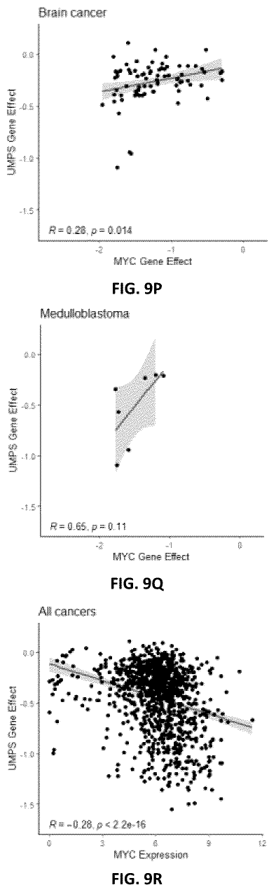

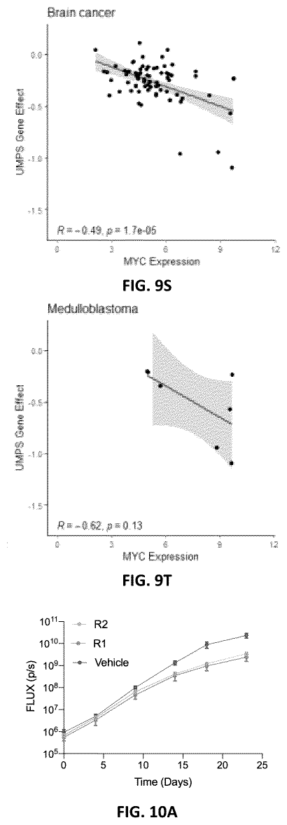

Based on the metabolite-protein interaction that is supported by this data and other studies^{44,45}, it was queried whether a dependency on de novo pyrimidine biosynthesis is a general feature of MYC-amplified cancers, irrespective of the tissue-of-origin. To explore this, gene dependency and gene expression profiles of 1,086 cancer cell lines from the DepMap were obtained. It was first noted that DHODH dependency exists on a spectrum across a broad range of tumor types yet is strongly selective in a subset of tumor subtypes (FIG. 9A). Indeed, pairwise comparisons demonstrate increased dependency for CAD, DHODH and UMPS in G3MB-MYC cell lines in comparison to all other brain tumor cell lines (FIG. 9B). Gene dependencies of CAD, DHODH and UMPS were negatively correlated with c-Myc expression (log₂ TPM) and positively correlated with c-Myc essentiality (FIG. 9C-T). While these associations are observed when analyzing all brain tumor profiles (n=78), they are particularly strong in medulloblastoma cell lines (n=7). Hence, whereas there are undeniably differences in contextual dependency on this pathway across different cancer types, this work identifies MYC-amplified group 3 MB as a particularly strong context.

Unbiased lipidomic profiling was used to explore other metabolic changes that occur in G3MB tumor cells upon DHODH inhibition. Drastic differences in the abundances of several lipid species were found, again with strong concordance between BAY-treated and DHODH KO tumor cells. Most notable were substantial increases in triglyceride and ether-linked phosphatidylethanolamine species (FIG. 4A). Such findings were consistent with genetic models of uridine deprivation (UPP2 knock-in), where uridine deprivation in hepatocytes leads to fatty liver steatosis via triglyceride accumulation⁴⁶. Together these data demonstrate that DHODH inhibition acts on target to disrupt uridine and lipid homeostasis, reducing the abundance of uridine metabolites and inducing a state of cellular hyperlipidemia.

If interference with uridine metabolism and lipid homeostasis is driving the cellular phenotype associated with DHODH deficiency, it was hypothesized that exogenous uridine ought to rescue such a phenotype in functional BTIC assays. Conversely, in contexts where the phenotype stems from a uridine-independent mechanism, such as blocking DHODH-mediated protection of tumor cells from ferroptosis, such a rescue does not occur⁴⁷. Indeed, it was determined that supplementation of cell culture media with uridine hydrochloride (100 μM) rescues SU_MB002 tumor cell proliferation and viability after pharmacological (with both BAY and BQR; FIG. 4D-E) inhibition of DHODH or DHODH KO (FIG. 4F-G). These data suggest that loss of BTIC activity in DHODH-deficient G3MB-MYC cells is primarily driven by uridine depletion, rather than noncanonical functions of DHODH.

### Example 6: Pharmacological Inhibition of DHODH Demonstrates Efficacy in a Preclinical PDOX Model of MYC-Amplified Group 3 Medulloblastoma

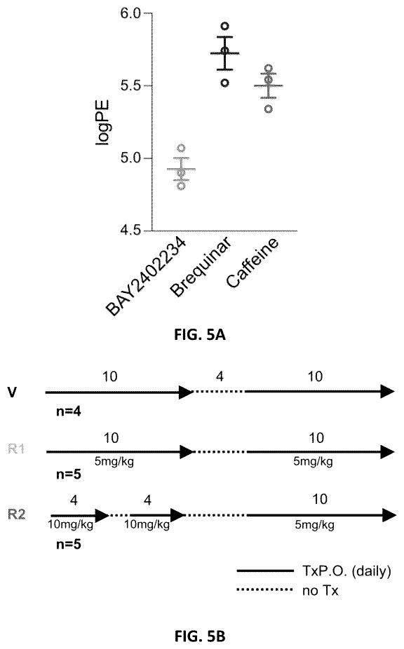

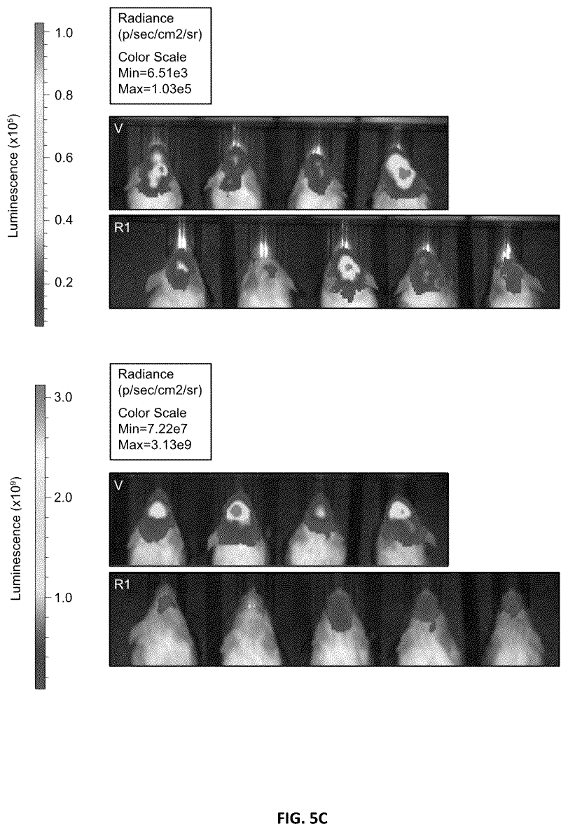

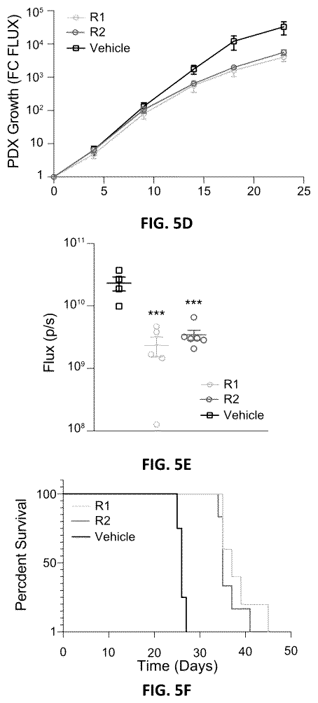

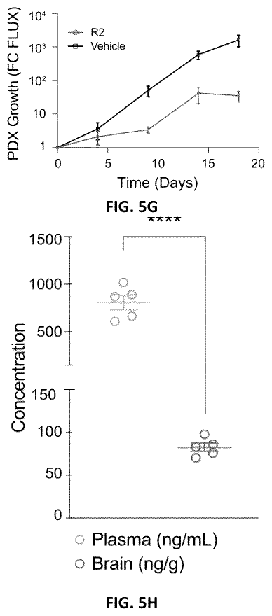

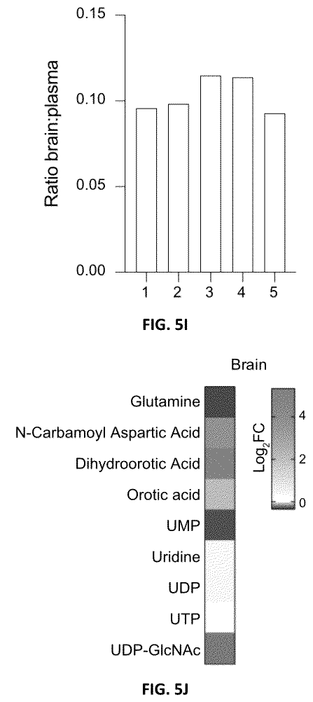

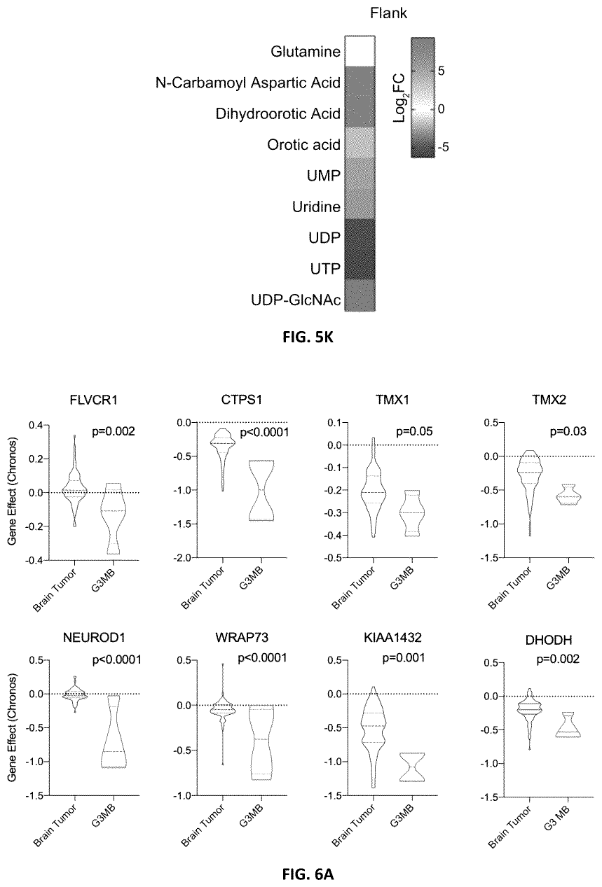

Given the selectivity, potency, and broad therapeutic window of DHODH inhibitors, it was queried whether such agents demonstrate in vivo efficacy against G3MB-MYC. The capacity of BQR and BAY to permeate the blood-brain barrier was first examined using the in vitro parallel artificial membrane permeability assay (PAMPA)⁴⁸ (FIG. 5A). In addition to enhanced oral bioavailability²⁵, BAY exhibited a superior penetration coefficient (log PE) compared to BQR, and was selected for in vivo preclinical study using an established PDOX model of G3MB-MYC.

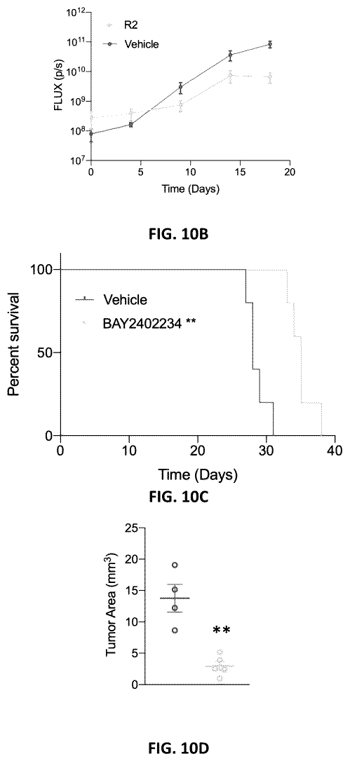

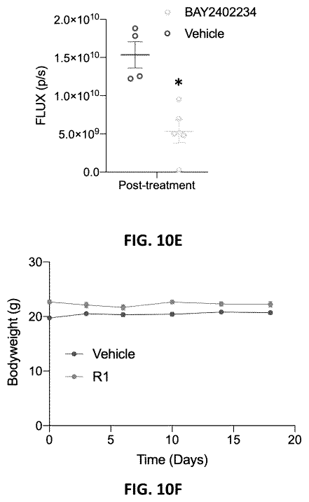

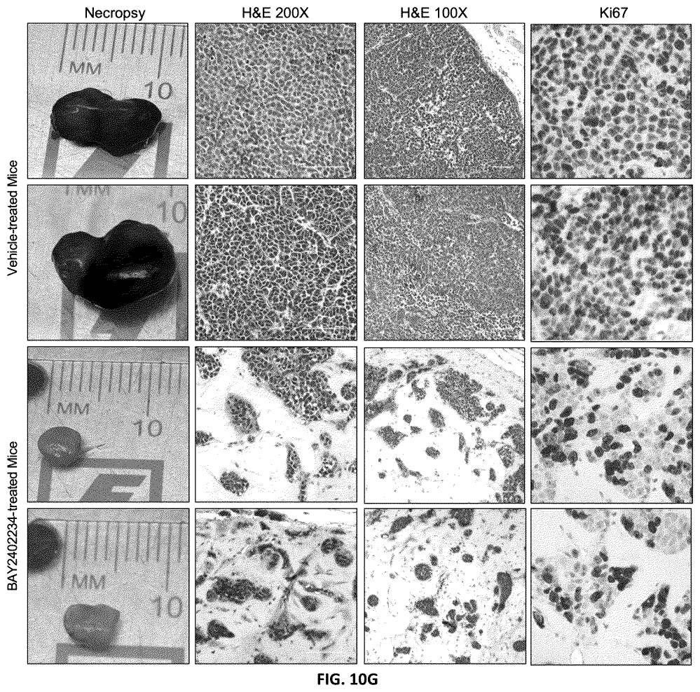

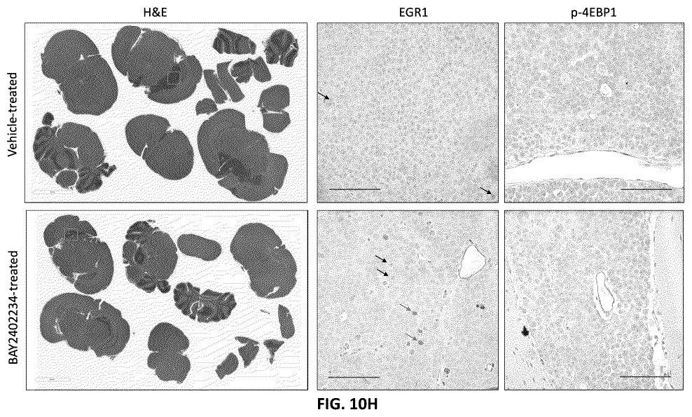

The treatment-naïve, patient-derived cell line, HD-MB03, was orthotopically engrafted into immunocompromised mice. Four days later, tumors were readily detectible via bioluminescence imaging. Thereafter mice were randomized into three cohorts, which were treated with either the vehicle or one of two BAY treatment regimens (R1 and R2; FIG. 5B). PDOX growth was significantly attenuated by both treatment regimens in comparison to those treated with the vehicle (FIG. 5C-E; S5A-B), leading to prolonged overall survival (FIG. 5F). The efficacy of BAY R1 was tested against orthotopic SU_MB002 tumors, demonstrating that reduced tumor burden and increased overall survival was reproducible across biological disease replicates (FIG. 10C-E). Notably, whereas 5 mg/kg was well-tolerated over the entire treatment period (FIG. 10F), mice that received R2 exhibited marginal, temporary weight loss after the 10 mg/kg doses. These data are consistent with the previously reported maximum tolerated doses of BAY²⁵.

In parallel to these experiments, subcutaneous flank xenografts were engrafted into two cohorts of mice, which were treated with the vehicle or R2. Interestingly, mice xenografted with HD-MB03 flank tumors exhibited a much stronger antitumoral response compared to those bearing orthotopic xenografts treated with the same dosing regimen (FIG. 5G). These data may in part reflect a limited capacity for BAY to penetrate the blood-brain barrier and accumulate to concentrations that approach or exceed the circulating plasma concentration. To investigate the pharmacokinetics of BAY in vivo, its concentration in plasma and brain tissues of healthy mice was directly quantified using an LC-MS approach. BAY was detected in both brains and plasma of mice undergoing treatment, however the plasma concentrations exceeded those of the brain by approximately 10-fold (FIG. 5H-I). To determine whether treatment with BAY affects de novo pyrimidine biosynthesis in vivo, changes in pyrimidine metabolism were measured in the brain in comparison to tumor tissues from the flank (FIG. 5J-K). Although BAY treatment impacted de novo pyrimidine metabolism in vivo in both brain and tumor tissues, with a similar pattern observed in vitro (FIG. 4A), the effect size was stronger in peripheral tumor tissue in comparison to brain tissue. Such findings further reflect the limited capacity for BAY to accumulate within the brain. Notably, UDP-GlcNAc abundance was reduced in both locations, suggesting that c-Myc activity could be impacted even at lower bioavailability.

To examine histological consequences of DHODH inhibition in vivo, the xenografts from the flanks of BAY- and vehicle-treated mice were surgically resected one day after administering the final dose. Notably, there was a profound difference in both tumor volume and visible vasculature between the treatment groups (FIG. 10G). Histological examination via H&E staining revealed striking morphological changes in xenografts from BAY-treated mice in comparison to vehicle-treated controls (FIG. 10G). Notably, tumor cell density was remarkably reduced in xenografts of BAY-treated mice in comparison to vehicle-treated mice, and many areas were completely devoid of tumor cells, having been replaced by fibrous, desmoplastic tissues. Immunohistochemistry (IHC) staining of residual xenograft tissues revealed that clusters of surviving tumor cells exhibited a reduction in Ki67 expression.

To determine whether mechanisms identified in vitro extend to the in vivo orthotopic model, time-matched PDOX were isolated from the brains of BAY-treated mice after the final dose of BAY was administered. IHC staining demonstrated that treatment with BAY decreased p-4EBP1 expression in comparison to vehicle-treated PDOX, consistent with reduced mTORC1 activity. An infrequent EGR1⁺ subpopulation of tumor cells was also detected, which varied in the levels of nuclear EGR1 expression (FIG. 10H). These cells were not detectable in the PDOX of vehicle-treated mice, suggesting that BAY treatment induced EGR1 expression. Together these data demonstrated that BAY exhibits in vivo preclinical efficacy in preclinical models of G3MB-MYC, with differential efficacy observed in PDOX in comparison to xenografts in the flank.

### Example 7: Discussion

Metabolic reprogramming is an emerging hallmark of cancer initiation and progression that is well-described across several hematological malignancies and solid tumors, including brain and lung neoplasms^{14,15,24,49-51}. For example, acute myeloid leukemia (AML)¹⁵ and glioblastoma (GBM)⁴⁹ stem cells exploit DHODH to promote tumor cell self-renewal and sustain disease burden. Similarly, hyperactivation of the pentose phosphate pathway enzyme PGD in GBM alleviates DNA damage from radiation therapy by quenching free radicals²⁴. The therapeutic potential for targetable metabolic dependencies in cancer has garnered significant clinical interest over the past decade, and whereas studies have suggested that the differential G3MB proteome (relative to other MB subgroups) reveals extensive metabolic reprograming⁵², the functional G3MB-MYC metabolome remains almost entirely unexplored.

This study used unbiased genetic screening and MS-based metabolomic profiling to discover several targetable metabolic vulnerabilities that have not been described in MB. These efforts led to the identification of PGD, SRM and DHODH as G3MB context-specific EGs. Whereas PGD and SRM represent prospective candidates for further therapeutic investigation, additional medicinal chemistry that can optimize highly selective, BBB-penetrant inhibitors is necessary. DHODH inhibitors, by contrast, represent likelier candidates for more immediate translation and were thus prioritized in this study.

This study explored de novo pyrimidine metabolism as a pleiotropic therapeutic vulnerability in G3MB-MYC BTICs. Analysis of scRNA-seq data from surgical G3MB specimens identified a corresponding MYC-amplified, pyrimidine-enriched phenotype in a subset of rapidly proliferating tumor cells with high phenotypic plasticity. G3MB-MYC BTICs with this phenotype can be selectively targeted by genetic or pharmacological inhibition of DHODH, both in vitro and in vivo. Such inhibition leads to mTORC1 inhibition, cell cycle arrest and apoptosis. Together, this multi-omic approach to drug discovery establishes a paradigm for targeting metabolic vulnerabilities in G3MB-MYC. Analysis of transcriptomic datasets, genome-wide CRISPR screens (in-house and via DepMap), and the differential potency of BAY against G3MB tumor cells in comparison to other cell types/MB subtypes further suggests that this pathway is contextually relevant in G3MB-MYC.

Analysis of metabolic changes associated with DHODH-deficient SU_MB002 tumor cells revealed significant changes in the levels of metabolites that are produced or consumed by DHODH enzymatic activity. Whereas DHODH dependencies have been described in IDH-mutant glioma and diffuse intrinsic pontine glioma (DIPG)^{53,54}, the connection between DHODH and c-Myc was not previously described. While these previous studies identified a connection to pyrimidine metabolites, the connection between pyrimidine and/or purine metabolites and MYC-amplification was not identified, as IDH-mutant glioma and DIPG are not myc-amplified cancers. In agreement with these reports, the complete phenotypic rescue mediated by uridine supplementation after DHODH inhibition suggests that de novo pyrimidine biosynthesis is a vulnerability due to depletion of UMP pools for cellular functions, and not due to a defective salvage pathway. The link between UDP-GlcNAc, O-GlcNAcylation and c-Myc stability, a metabolite-protein interaction that has been reported in hematological malignancies^{44,45}, might in part explain the context-specificity of this vulnerability in G3MB. Interestingly, mTORC1 is also the target of O-GlcNAcylation in cancer⁵⁵.

Lipidomic profiling of DHODH-deficient tumor cells revealed staggering increases in the abundance of several lipid species relative to their respective controls, most notably triglycerides and phosphatidylethanolamines. Such increases in lipid abundance are in keeping with a role for uridine metabolism in lipid homeostasis, and the rapid accumulation of triglycerides caused by uridine depletion in fatty liver disease⁴⁶. Notably, excess triglyceride abundance has also been reported to activate toxic cellular responses including EGR-1-directed transcriptional programs such as TNFα via NF-κB⁵⁶. Other studies have linked altered phosphatidylethanolamine abundance to mitochondrial dysfunction, neuronal differentiation and ferroptosis⁵⁷. The speculation that such processes are related to these findings warrants investigation into the functional consequences of hyperlipidemia following DHODH inhibition.

This study highlights an association between EGR1 enrichment and mechanisms of cell death following DHODH inhibition. It was determined that EGR1 regulon is negatively correlated with that of c-Myc in G3MB surgical specimens, and that this transcriptional program is active alongside known c-Jun and ERK1/2 induction mechanisms. It is speculated that apoptosis and cell cycle arrest programs in DHODH-deficient tumor cells may reflect a switch from a MYC-regulated, rapidly cycling phenotype to one that is EGR1-regulated and tumor-suppressive. The intersection between ERK2-mediated suppression of mTORC1, and induction of NF-κB/EGR1 signaling to drive tumor cell apoptosis has been observed in leukemic tumor cells treated with combinatorial mTOR inhibitors⁵⁸. Whereas these associations are compelling, whether EGR1 is casually linked with metabolic stress responses in G3MB requires further study. The recently described role of EGR1 in inducing hypomethylation of genes driving neuronal differentiation in the postnatal brain⁴² is especially interesting from a developmental biology perspective, given that G3MB is thought to arise from a NSC gone awry^{16,59,60}.

Clinical investigations of orally formulated BAY2402234 safety and efficacy have been reported in AML [NCT03404726] and more recently, in recurrent glioma [NCT05061251]. The notion that DHODH inhibitors possess such potent in vivo efficacy but a seemingly minimal effect on the viability of NSCs cultured in vitro is an exciting prospect. Such treatments may allow de-escalation of neurotoxic chemo- or radiotherapies that reduce the neurotoxic sequelae associated with systemic cytotoxic therapies, thus improving patient quality-of-life through the integration of a unique treatment paradigm.

### Example 8: Materials and Methods

### Cell Culture

SU_MB002, NSC194, NSC197 and NSC201FT, MBT375 and MED411FHTC were maintained in Neurocult Complete (NCC) media (Stem Cell Technologies; catalogue #05751). D425 and D458 were maintained in Dulbecco's Minimum Essential Media (DMEM) containing 10% fetal bovine serum (FBS). HD-MB03 were maintained in NCC containing 10% FBS. All cell lines were conditioned in NCC for a minimum of 48 hours before experimentation. SU_MB002 and HD-MB03 G3MB-MYC PDOX were obtained from collaborators as kind gifts and are frequently passaged in vivo. SU_MB002, a treatment-refractory group 3 MB acquired at autopsy was received from Dr. Yoon-Jae Cho. Dr. Till Milde provided HD-MB03, a treatment-naïve group 3 MBs. ICB1299 group 4 MB cells were obtained as a gift from Silvia Marino's laboratory⁶². MBT375 was established in-house from a surgical specimen of group 4 MB, which was engrafted in the cerebellum of mice for serial propagation. Nanostring genotyping was used to confirm group 4 medulloblastoma identity. MED411FHTC PDOX were procured from Dr. Olson's brain tumor research lab. D425 and D458 were obtained from the ATCC. All cell lines were fingerprint verified by short tandem repeat (STR) profiling. NSC194, NSC197 and NSC201FT were derived in-house from donor fetal brain tissues⁶³.

### Cloning of CRISPR-Cas9 Constructs

Individual sgRNA sequences were cloned into lentiCRISPRv2 plasmids as described previously⁶⁴. All constructs were sequenced by Sanger sequencing to confirm successful integration.

### Western Blotting

Protein lysates were isolated from approximately 500,000-1,000,000 cells. Pellets were rinsed with PBS and then lysed in RIPA buffer containing HALT™ protease inhibitor cocktail (ThermoFisher). Protein concentrations were quantified using the Bradford Assay (BioRad). Protein size was resolved via SDS-PAGE and then transferred to PVDF for immunoblotting. Rabbit polyclonal anti-DHODH (Cell Signaling Technologies; catalogue #80981), rabbit monoclonal anti-EGR1 (Cell Signaling Technologies; catalogue #4145), rabbit polyclonal anti-phospho-ERK1/2 Thr202/Tyr204 (Cell Signaling Technologies; catalogue #9101), rabbit monoclonal anti-phospho-4E-BP1 Thr37/46 (Cell Signaling Technologies; catalogue #2855) were used at a 1:1000 dilution. Mouse monoclonal anti-GAPDH (Abcam; catalogue #ab8245) was used at a 1:5000 dilution. For development, horse-radish peroxidase-conjugated secondary anti-mouse (BioRad; catalogue #1706516) and anti-rabbit antibodies (BioRad; catalogue #1706515) were used with BioRad Clarity ECL reagents. Quantification was performed by analyzing mean pixel density of each band and then normalizing to GAPDH loading control. The proteome profiler array was obtained from R&D systems (catalogue ARY003C) and performed according to manufacturing guidelines, using 400 μg cellular lysate for each sample.

### Immunohistochemistry

FFPE tissues were sectioned onto slides and then deparaffinized prior to analysis. Samples were stained with H&E for histological examination. In parallel, sections were blocked in 5% BSA (anti-Ki67, anti-4E-BP1 Thr37/46, anti-DHODH) or 5% NGS (anti-EGR1) and stained with primary antibody at 4° C. overnight. Rabbit anti-Ki67 (Thermo Fisher Scientific; Catalogue #RM-9106-S0) was diluted 1:100, rabbit anti-EGR1 (Cell Signaling Technologies; catalogue #4145) was diluted 1:200, rabbit anti-4E-BP1 Thr37/46 (Cell Signaling Technologies; catalogue #2855) was diluted 1:1000, and rabbit anti-DHODH (Proteintech 14877-1-AP) was diluted 1:200. Anti-rabbit HRP-conjugated secondary antibodies (Abcam; Catalogue #ab214880) were used with DAB for development (Vector Laboratories; Catalogue #SK-4100). All slides were scanned using Image Scope (Aperio).

### RNA Sequencing

Total RNA was isolated from 200,000-300,000 viable tumor cells and then sequenced using Illumina HiSeq. RNA sequencing reads were trimmed and filtered using BBDuk from the BBTools package⁶⁵. Sequencing reads were aligned using STAR aligner version 2.6.0 and Ensembl reference genome GRC38. Expression levels were quantified as TMM based using Edge® 3.26.4 using the ‘calcNormFactors’ and ‘cpm’ functions. The pre-processing pipeline is published and freely available on GitHub for reproducibility. Differential gene expression analysis was performed using Limma with Benjamini and Hochberg correction for multiple comparisons. Read counts less than 10 were filtered prior to analysis. Gene set enrichment analysis was carried out using the GSEAv4.2.1 (Broad Institute)³⁶. Gene sets enriched with FDR<0.05 and p<0.05 were considered statistically significant.

### In Vitro Functional BTIC Assays

For assessment of viability, cells were seeded into 96-well plates at a density of 1,000 cells per well. Viability was measured by adding PrestoBlue™ diluted 1:10 (ThermoFisher Scientific; Catalogue #A13261) followed by a 4-hour incubation. Relative fluorescence intensity was determined via background correction. To assess tumorsphere forming capacity, cells were seeded at a low density (200 cells per well) and incubated for 3-5 days. Tumorspheres were manually counted thereafter.

### Cell Cycle and Apoptosis Analysis Via FACS

Detection of apoptotic cells was FITC Annexin V/PI (Thermo Fisher Scientific; Catalogue #A23204) using manufacturer's protocol. Briefly, 100,000 cells/sample were washed twice with PBS and resuspended in 100 μL binding buffer (Thermo Fisher Scientific; Catalogue #V13246) and incubated with Annexin V/PI and vortexed. Sample was then incubated in the dark for 15 min before addition of 400 μL of binding buffer and analyzed. Cell cycle was assessed using the Coulter DNA Prep Reagents Kit (Beckman Coulter; Catalogue #6607055) following manufacturer's protocol, briefly 200,000 cells/sample in 50 μL PBS were incubated with equal volume of DNA PREP LPR, vortexed vigorously for 10s, and incubated with 750 μL of DNA PREP Stain and vortexed again for 10s and incubated at room temperature for 15 min before analysis. All flow cytometry analysis was performed on the CytoFLEX LX flow cytometer.

### Single Cell RNA Seq Analysis

Preprocessing. Raw counts for publicly available scRNA Seq were obtained through accession code GSE119926³¹. The Seurat V4 analysis pipeline (R package, v4.1.2⁶⁶) was implemented as previously described^{67,68}. Data integration steps described by Stuart et al⁶⁶ consisted of normalization (SCTransform) and identification of 3000 “anchors” features subsequently used in the iterative pairwise integration (CCA integration). Integrated counts were embedded in two-dimensional space using Uniform Manifold Approximation and Projection (UMAP)⁶⁹ with default parameters and Louvain clustered using FindClusters( . . . , resolution=0.8) (Seurat)

Metagene scores. Metagene scores of genes known to associate with a biological process (Kyoto Encyclopedia of Genes and Genome, KEGG⁷⁰) were used to quantify the activity of that biological program of interest. Metagene scores were computed using AddModuleScore( . . . ) (Seurat) which calculates the difference between the centered, log-transformed mean counts of an input gene list and the centered, log-transformed mean counts of the remaining genes as described²⁸. Cellular differentiation potential was computed using CytoTRACE (R package, v0.3.3³²).

Regulon Analysis. Regulons are gene programs with significant motif enrichment for an upstream transcription factor, and the metagene score for a given regulon offers a readout of the corresponding transcription factor's activity. To infer regulons from the scRNA-seq data, a modified implementation of SCENIC (R package, v1.2.2⁷¹) was used. In brief, genes expressed in >10% of cells within at least one cluster [clusters derived defined above using FindClusters( . . . )] were retained and used to generate a gene×gene similarity matrix using Spearman correlation. The similarity matrix was converted to a sorted list of regulatory links using getLinkList( . . . ) (GENIE3 R package, v1.12.0⁷²) from which co-expression gene modules were then derived using runSCENIC_1_coexNetwork2modules( . . . ). Co-expression modules were pruned using TF-motif enrichment analysis [RcisTarget (R package, v1.8.0)] via SCENIC's implementation with runSCENIC_2_createRegulons( . . . , coexMethods=“top1sd”). Finally, regulon activity was computed by running AddModuleScore( . . . ) on each of the resulting regulon genesets, and the correlation of each regulon with its corresponding transcription factor's expression was evaluated. Regulon-associated genesets were functionally annotated using hypergeometric analysis using fora (fgsea R package, v 1.14.0⁷³). Annotated gene sets used for enrichment analyses included gene ontology (GO; biological processes, cellular components, molecular function) and gene-set collections (including HALLMARK, PID, MSigDB, Reactome, and others) curated by the Bader Lab⁷⁴.

### Proximal CpG Analysis

The relationship between an EGR regulon-associated genes and CpG-associated genomic features was evaluated using the annotatr (R package v1.14.0⁷⁵). CpG-associated genomic features included CpG islands (CGI), CpG shores (2 Kb upstream/downstream from ends of CpG islands), CpG shelves (2 Kb upstream/downstream of the farthest upstream/downstream limits of CpG shores), and inter-CGI (genomic regions not covered by CpG islands, CpG shores, or CpG shelves). Genomic annotations were prepared using build_annotations(genome=‘hg38’, annotations=c(‘hg38_cpgs’, ‘hg38_basicgenes’)) and genomic regions associated with the EGR regulon genes and CpG features were identified using annotate_regions( . . . ) with default parameters. To evaluate whether the association between the genes and CpG features was significant, the association between random genes and CpG features was evaluated over 1000 permutations, and the resulting null distribution was used to obtain a p-value from the Z score calculated from the scaled difference between observed and expected CpG feature-associated gene counts. Results are expressed as a CpG enrichment ratio, which is the ratio of observed to expected CpG feature-associated gene counts.

### Differential Methylation Analysis

To interrogate the differences in methylation after DHODH inhibition, SU_MB002 cells were treated with DMSO or BAY (IC₅₀) and collected at 48 h and 72 h (2 technical replicates per condition). gDNA was extracted using Puregene Cell and Tissue kit (Qiagen) as per manufacturer's instructions and quantified using spectrophotometry. Illumina Human Infinium MethylationEPIC (EPIC) BeadChip (Illumina, CA, USA) array was performed at The Centre for Applied Genomics, The Hospital for Sick Children (Toronto) using the manufacturer's standard protocol.

Raw IDAT files (annotation: ilm10b4.hg19) were processed using R (Version 4.1.2) and quality control and normalization was performed using minfi⁷⁶. Intensity and density plots were generated, and no samples were removed from the analysis as the mean detection p-values for all probed genomic positions were <0.01 for each sample. Probes with mean detection p-values >0.01 across all samples were excluded. Probes mapping sex chromosomes, single nucleotide polymorphisms (SNPs), and cross-hybridisation probes were removed. Quantile normalization was performed, and density plots were generated before and after normalization. Differential analysis was completed with beta values of 768467 CpGs for 8 samples comparing BAY to DMSO at 48 h and 72 h using limma⁷⁷. P-values were adjusted for False Discovery Rate (FDR) using Benjamini and Hochberg method, and a 10% FDR and log FC of >|0.1| cut-off was used.

Differentially methylated positions (DMPs) were annotated using the USCS (UCSC_RefGene_Name, UCSC_RefGene_Group and Relation_to_Island) from the Infinium MethylationEPIC Manifest. The analysis was performed focusing on DMPs within promoter islands (considering TSS1500, TSS200, 5′UTR and 1st exon as promotor regions). Enrichment analysis of unique genes (using UCSC RefGene Name) identified within DMPs at promoter islands was performed using Enrichr (ENCODE TF ChIP-seq 2015)⁴³.

### In Vivo Studies

PDOX were generated by engrafting 25,000 tumor cells (both SU_MB002 and HD-MB03 cell lines), suspended in 5 μL PBS, directly into the mouse cerebrum. Flank xenografts were generated by engrafting 2,000,000 tumor cells suspended in a 100 μL solution of PBS/Matrigel® (1:1 v/v). For preclinical studies, cells were engineered to express enhanced firefly luciferase. BAY2402234 was administered according to regimens listed in FIG. 5B by oral gavage in a 100 μL volume. Flank tumors were isolated at matched time points and formalin fixed for 48 hours, paraffin embedded (FFPE) and sectioned for follow-up analysis. Tumor area was manually measured in H&E-stained sections of FFPE tissues using ImageJ.

### Pharmacokinetic Analysis of BAY2402234

Standards and samples were extracted by the addition of organic solvent, containing the internal standard (IS), for protein precipitation. Brain samples were homogenized with Dulbecco Phosphate Buffered Saline (DPBS) in a ratio of 1:2 (w/V, brain:DPBS) using Qiagen tissue ruptor probes prior to the addition of organic solvent containing the IS. For plasma analysis, 70 μL of IS working solution was added to 1.5 mL Eppendorf tubes, then 5 μL of standard or unknown sample was transferred to the tube. Samples were vortexed for 2 min and then centrifuged at 15,000 g for 5 min at 4° C. Next, 45 μL of 40% MP-B was added to HPLC vials and 5 μL of supernatant was transferred to the designated HPLC vial, and then vortexed for 30 sec. 10 μL was then injected for LC-MS analysis. Brains were analyzed similarly, with 20 μL of sample transferred to 180 μL of IS solution prior to analysis via LC-MS.

### In Vivo Imaging System (IVIS)

For imaging via the in vivo imaging system (IVIS), mice were sedated with isoflurane gas and then administered luciferin substrate (150 mg/kg in PBS) via subcutaneous injection. After 10 minutes, bioluminescent signal (photons per second; p/s) was quantified using Living Image software.

### Compounds

BAY2402234 was purchased from Cayman Chemical Suppliers (Catalogue #33259), Brequinar sodium was purchased from Tocris (Catalogue #6196), and PTC299 and PTC868 were procured via a scientific research agreement with PTC Therapeutics (PTC Therapeutics, Inc. 100 Corporate Court, South Plainfield, NJ 07080). PTC299 is also commercially available from Caymen Chemicals (Item No. 36877). Uridine hydrochloride was purchased from Cayman Chemical Suppliers (Catalogue #20300). Compounds were dissolved in DMSO for in vitro assays. For in vivo studies, BAY2402234 was dissolved in polyethylene glycol (80%) and ethanol (20%).

### CRISPR-Cas9 Screening

SU_MB002 tumor cells were infected with TKOv3.0 library at a multiplicity of infection (MOI) of 0.3, ensuring a single vector integration event per cell. Genomic DNA was isolated after puromycin selection (TO) and again after 30 days of growth in vitro (T30).

Sequencing libraries were sequenced on an Illumina HiSeq2500 using single-read sequencing and were completed with standard primers for dual indexing with HiSeq SBS Kit v4 reagents. The first 21 cycles of sequencing were dark cycles, or base additions without imaging. The actual 36-base read begins after the dark cycles and contains 2 index reads, in which i7 is read first, followed by the i5 sequences. The TO and late time point samples were sequenced at 400- and 200-fold library coverage, respectively. Reads from each sample were aligned to the TKOv3 library FASTA file with bowtie (v0.12.8) allowing two mismatches (−v2) and discarding any read that mapped to more than one sequence in the library (−m1). Reads mapping to each sgRNA were summed and merged into a read count matrix, along with guide-level annotations.

The count matrix for each screen was processed using the R statistical computing environment as follows. First, guides with fewer than 30 reads in TO samples were filtered out, and NA values representing guides missing in individual samples were replaced with zeros. Next, normalization factors for each sample were determined using the calcNormFactors( ) function, guide-level log 2-fold-change values were determined using the Limma package (v3.46.0), and significantly enriched or depleted genes were identified using the camera( ) function. Gene-level log-fold-change values were determined by averaging sgRNA measurements for genes targeted by two or more sgRNAs.

Gene-level BAYES factor (BF) scores were calculated for each gene using BAGEL²¹. SU_MB002-selective EGs were identified by integrating EGs in SU_MB002 tumor cells (BF greater than 5; FDR<0.05) with those identified in NSC by Toledo et al. Genes with a BF of greater than 5 in either U5 or CB66 were filtered as NSC EGs. Mean log₂ fc of sgRNAs targeting each SU_MB002-selective essential gene were calculated for each cell line and displayed in the heatmap shown in FIG. 1B.

### Mass Spectrometry-Based Metabolomics and Lipidomics Analyses

For metabolome and lipidome profiling, ˜10⁶ cells and tissues were washed with PBS prior to collection and immediately flash frozen. For metabolite extraction, 1 mL of cold acetonitrile/methanol/water (2:2:1; v:v:v) was added to the cell pellets followed by 3 cycles of freezing in liquid N2 for 1 min, followed by 15 min of thawing in a sonicating ice/water bath. This was followed by incubation at −20° C. and centrifugation at 16,000×g for 15 min at 4° C. 1 hour to fully precipitate protein. Supernatants were transferred to another tube and dried in a vacuum concentrator. For lipid extraction, cells were resuspended in 30 μL of water, followed by three cycles of freeze/thawing and sonication. To this, 225 μL of methanol was added, followed by brief vortexing and addition of 750 μL of methyl tert-butyl ether (MTBE). Samples were incubated at room temperature for 1 hr with gentle shaking. To induce phase-separation, 188 μL of water was added, samples were vortexed for 20 s and centrifuged for 5 min at 16,000×g. The upper and lower phases were collected and transferred to another tube and dried down in a vacuum concentrator. The dry metabolome and lipidome extracts were stored at −80° C. prior to analysis by liquid chromatography-mass spectrometry (LC-MS). Metabolite extracts were reconstituted in LC-MS grade water/acetonitrile (50:50; v:v), vortexed for 1 min, sonicated for 15 min, and centrifuged at 16,000×g and 4° C. for 15 min to remove insoluble debris. The lipidome extracts were reconstituted in acetonitrile/2-propanol (50:50; v:v), vortexed for 1 min, sonicated for 15 min, and centrifuged at 16,000×g and 10° C. for 15 min to remove insoluble debris.

For metabolome profiling of tissues, ˜5 mg tissue were homogenized in 225 μL of −20° C. cold, methanol using a Bullet Blender Tissue Homogenizer (NextAdvance, Inc.) Bullet for 3 min. The homogenate was vortexed for 20 s and 750 μL of −20° C. cold methyl tertiary-butyl ether (MTBE) was added, and the mixture was vortexed for 10 s and shaken at 4° C. for 5 min. Next, 188 μL room temperature water was added and vortexed for 20 s to induce phase separation. After centrifugation for 5 min at 14,000×g, the bottom polar phase was collected and dried down. Metabolite extracts were reconstituted in LC-MS grade water/acetonitrile (50:50; v:v), vortexed for 1 min, sonicated for 15 min, and centrifuged at 16,000×g and 4° C. for 15 min to remove insoluble debris.

The LC-MS metabolomics and lipidomics analyses of the 3 NSC and 3 MB cell lines and tissues were performed on a UPLC-MS system consisting of an Orbitrap IQ-X Tribrid coupled to a Horizon Vanquish UPLC system (Thermo Fisher Scientific). The lipidomics analysis was conducted in positive-ion mode with a spray voltage of 3.5 kV while the metabolomics analysis was conducted in negative-ion mode with a spray voltage of −2.5 kV. The following source conditions were identical in both analyses and were set as follows: Sheath gas flow rate, 50 arbitrary units; Aux. gas flow rate, 10 arbitrary units; Sweep gas flow rate, 1 arbitrary unit; Ion transfer tube temp., 300° C.; Vaporizer temp., 350° C. The following acquisition parameters were used for MS1 analysis in both methods: resolution, 120,000, AGC target, 4e5. The maximum IT was set at 50 s for the lipidomics analysis and 200 ms for the metabolomics analysis. Data-dependent MS/MS parameters for the lipidomics analyses: resolution, 30,000; AGC target, 7.5e4; maximum IT, Dynamic (54 ms); time between Master Scans, 0.6 s; isolation window, 1.0 m/z; NCE/stepped nce, 20, 30, 40; intensity threshold, 5e4; exclude isotopes, on; dynamic exclusion, 3.0 s. Data-dependent MS/MS parameters for the metabolomics analyses: resolution. 15,000; AGC target, 7.5e4; maximum IT, Dynamic (22 ms); time between Master Scans, 0.6 s; isolation window, 1.0 m/z; NCE/stepped nce, 20, 30, 40, 50; intensity threshold, 5e4; exclude isotopes, on; dynamic exclusion, 5.0 s. To increase the total number of MS/MS spectra in the lipidomics analysis, five runs with iterative MS/MS exclusions were performed using the AcquireX function in Xcalibur (Thermo Fisher Scientific). In the metabolomics analysis, three runs with iterative MS/MS exclusions were performed using the AcquireX function. For the lipidomics analysis, chromatographic separation was achieved using a Waters Acquity UPLC CSH C18 (100 mm×2.1 mm i.d.; 1.7 μm) column injecting 2 μL. The mobile phases consisted of acetonitrile/water (60:40, v/v) with 0.1% formic acid and 10 mM ammonium formate as A and 2-propanol/acetonitrile (90:10, v/v) with 0.1% formic acid and 10 mM ammonium formate as B. The gradient employed was as follows: 0-2 min 15% B, 2-2.5 min increase to 30% B, 2.5-11 min to 82% B and finally 11-11.5 min to 99% B and held for 30 s. The column was equilibrated for 2 min at 15% B. The flow rate was set to 250 μL/min. The column temperature was maintained at 65° C. For the metabolomics analysis, chromatographic separation was achieved using a SeQuant ZIC-PHILIC (150 mm×2.1 mm i.d.; 5 μm) column injecting 2 μL. The mobile phases consisted of water/acetonitrile (95:5, v/v) with 25 mM ammonium bicarbonate, 0.1% ammonium hydroxide, and 2.5 μM medronic acid as A and acetonitrile/water (95:5, v/v) with 2.5 μM medronic acid as B. The gradient employed was as follows: 0-1 min 90% B, 1-14 min decrease to 25% B, 14-14.5 min 25% B, 15 min to 90% B and held until 22 min. The flow rate was set to 250 μL/min. The column temperature was maintained at 40° C.

The LC-MS metabolomics analysis for SU_MB002 DHODH KO and cells treated with BAY2402234 inhibitor was performed on a UPLC-MS system consisting of an Agilent 6550 qToF coupled to an Agilent 1290 binary pump UPLC system. The source parameters were as follows: Gas temperature, 150° C. at 14 L/min and 45 psig; Sheath gas temperature, 325° C. at 12 L/min; Capillary and nozzle voltages were set to −2.0 kV. iFunnel conditions were changed from default to −30 V DC, High pressure funnel drop −100 V and RF voltage of 110 V, low pressure funnel drop-50 V and RF voltage of 60 V. Chromatographic separation was achieved by ion-paired chromatography. In brief, 2 μL of each sample was injected onto Agilent ZORBAX Extend-C18 (150 mm×2.1 mm i.d.; 1.8 μm) column using tributylamine (TBA) as an ion paring agent (solvent A: 3% methanol, 97% water 10 mM TBA, 15 mM Acetic acid, solvent B: 100% methanol). The linear gradient employed was as follows: 0-2.5 min 99% A, 2.5-7.5 min decrease to 80% A, 7.5-13 min to 55% B and finally 13-15 min to 99% B and held for 1 minute. The column was regenerated for 2 min at 1% B. The flow rate was set to 250 μL/min. The column temperature was maintained at 25° C. The lipidomics analysis was performed as described above.

### Statistical Analysis

All experiments were performed twice to ensure reproducibility. A minimum of four technical replicates were used for mean comparisons. Pairwise differences were measured using two-tailed independent student's t-tests. When more than two groups were compared, One-way analysis of variance (ANOVA) was used followed by post-hoc Tukey's tests for individual comparisons. For in vivo studies, median survival differences were measured using Kaplan-Meier survival analysis, and significance was determine using the Log-rank test. Metabolomics differences were assessed for significance by multiple t-tests, using a two-stage linear step-up procedure of Benjamini, Krieger and Yekutieli, with Q<0.01.

### Ethics Approvals

All in vivo experiments were carried out in accordance with the Canadian Council on Animal Care (CCAC) under animal utilization protocol (19-01-01) approved by the Animal Research Ethics Board (AREB). Human tissues were isolated using protocols approved by the Human Integrated Research Ethics Board (HIREB).

### Example 9: Metabolite Abundance is Correlated with DHODH Essentiality in Myc-Amplified Cancers

Nucleotide sugar monophosphates predict DHODH essentiality and identify MYC-expressing tumors amenable to DHODH inhibition therapy.

Due to the association between increased abundance of UMP, CMP, AMP, and GMP (nucleotide sugars) in MYC-amplified group 3 medulloblastoma and the sensitivity of these cells to DHODH inhibition (in comparison to NSC), the present inventors examined whether these metabolites act as biomarkers for predicting response to DHODH inhibition. This was explored using publicly available transcriptomic, functional genomic, and metabolomic data for a broad range of cancer types (DepMap). The results show that the same four nucleotide sugars (AMP, UMP, CMP, and GMP) were the top-ranking metabolites whose relative increased abundance associated most strongly with DHODH ‘essentiality’ (Table 1). This analysis was broadened to look at MYC expression as well, as the work shows that UMP-derived UDP-GlcNAc stabilizes c-Myc at the protein level. Indeed, UMP was the highest-ranking metabolite that correlates with high c-Myc transcript expression (Table 2).

Cancer types that were included in the DepMap analysis of metabolite abundance and DHODH essentiality (Table 1) include: Acute Myeloid Leukemia, Adenosquamous Carcinoma of the Pancreas, Ampullary Carcinoma, Anaplastic Thyroid Cancer, B-Lymphoblastic Leukemia/Lymphoma, Bladder Squamous Cell Carcinoma, Bladder Urothelial Carcinoma, Breast Ductal Carcinoma In Situ, Chondrosarcoma, Colorectal Adenocarcinoma, Diffuse Glioma, Embryonal Tumor, Endometrial Carcinoma, Esophageal Squamous Cell Carcinoma, Esophagogastric Adenocarcinoma, Ewing Sarcoma, Fibrosarcoma, Head and Neck Squamous Cell Carcinoma, Hepatoblastoma, Hepatocellular Carcinoma, Hodgkin Lymphoma, Intracholecystic Papillary Neoplasm, Intraductal Papillary Neoplasm of the Bile Duct, Invasive Breast Carcinoma, Leiomyosarcoma, Lung Neuroendocrine Tumor, Melanoma, Myelodysplastic Syndromes, Myeloproliferative Neoplasms, Neuroblastoma, Hodgkin Lymphoma, Non-Small Cell Lung Cancer, Osteosarcoma, Ovarian Epithelial Tumor, Pancreatic Adenocarcinoma [symbol] Pleural Mesothelioma [symbol] Poorly Differentiated Thyroid Cancer, Prostate Adenocarcinoma, Renal Cell Carcinoma, Rhabdomvosarcoma, Sarcoma, NOS, T-Lymphoblastic Leukemia/Lymphoma, Undifferentiated Pleomorphic Sarcoma/Malignant Fibrous Histiocytoma/High-Grade Spindle Cell Sarcoma, Urethral Cancer, Uterine Sarcoma/Mesenchymal, Well-Differentiated Thyroid Cancer.

Cancer types that were included in the DepMap analysis of metabolite abundance and c-Myc transcript expression (Table 2) include: Acute Myeloid Leukemia, Adenosquamous Carcinoma of the Pancreas, Ampullary Carcinoma, Anaplastic Thyroid Cancer, B-Lymphoblastic Leukemia/Lymphoma, Bladder Squamous Cell Carcinoma, Bladder Urothelial Carcinoma, Breast Ductal Carcinoma In Situ, Chondrosarcoma, Colorectal Adenocarcinoma, Diffuse Glioma, Embryonal Tumor Endometrial Carcinoma, Esophageal Squamous Cell Carcinoma, Esophagogastric Adenocarcinoma, Ewing Sarcoma, Fibrosarcoma, Head and Neck Squamous Cell Carcinoma, Hepatoblastoma, Hepatocellular Carcinoma, Hodgkin Lymphoma, Intracholecystic Papillary Neoplasm, Intraductal Papillary Neoplasm of the Bile Duct, Invasive Breast Carcinoma, Leiomyosarcoma, Lung Neuroendocrine Tumor, Medullary Thyroid Cancer, Melanoma, Myelodysplastic Syndromes, Myeloproliferative Neoplasms, Neuroblastoma, Non-Cancerous, Non-Hodgkin Lymphoma, Non-Small Cell Lung Cancer, Osteosarcoma, Ovarian Epithelial Tumor, Pancreatic Adenocarcinoma, Pancreatic Neuroendocrine Tumor, Pleural Mesothelioma Poorly Differentiated Thyroid Cancer, Prostate Adenocarcinoma, Prostate Small Cell Carcinoma Renal Cell Carcinoma, Rhabdoid Cancer, Rhabdomyosarcoma, Sarcoma, NOS, T-Lymphoblastic Leukemia/Lymphoma, Undifferentiated Pleomorphic Sarcoma/Malignant Fibrous Histiocytoma/High-Grade Spindle Cell Sarcoma, Urethral Cancer, Uterine Sarcoma/Mesenchymal, Well-Differentiated Thyroid Cancer.

Based on the data from Table 1 and Table 2, cancer types in which DHODH was determined to be essential include: Acute Myeloid Leukemia, Ampullary Carcinoma, B-Lymphoblastic Leukemia/Lymphoma, Bladder Urothelial Carcinoma, Breast Ductal Carcinoma In Situ, Breast Neoplasm, NOS, Cervical Squamous Cell Carcinoma, Colorectal Adenocarcinoma, Diffuse Glioma, Embryonal Tumor, Endometrial Carcinoma, Esophageal Squamous Cell Carcinoma, Esophagogastric Adenocarcinoma, Ewing Sarcoma, Extra Gonadal Germ Cell Tumor, Head and Neck Carcinoma, Other, Head and Neck Squamous Cell Carcinoma, Hepatocellular Carcinoma, Hepatocellular Carcinoma plus Intrahepatic Cholangiocarcinoma, Intracholecystic Papillary Neoplasm, Intraductal Papillary Neoplasm of the Bile Duct, Invasive Breast Carcinoma, Leiomyosarcoma, Lung Neuroendocrine Tumor, Melanoma, Meningothelial Tumor, Myelodysplastic Syndromes, Myeloproliferative Neoplasms, Neuroblastoma, Non-Hodgkin Lymphoma, Non-Small Cell Lung Cancer, Ocular Melanoma, Ovarian Epithelial Tumor, Ovarian Germ Cell Tumor, Pancreatic Adenocarcinoma, Pleural Mesothelioma, Prostate Adenocarcinoma, Renal Cell Carcinoma, Rhabdomyosarcoma, Synovial Sarcoma, T-Lymphoblastic Leukemia/Lymphoma, Urethral Cancer, Well-Differentiated Thyroid Cancer.

TABLE 1
Unbiased analysis of DHODH gene essentiality (Gene Effect)
versus metabolite abundance (log2 abundance) in 625 cell
lines. Data obtained via public DepMap.org portal. Rank
ordered by Pearson's correlation metric (Cor).

| Cor | PValue | QValue | Metabolite |
|---|---|---|---|
| −0.4874517 | 1.13E−38 | 2.54E−36 | AMP |
| −0.4842416 | 4.07E−38 | 4.58E−36 | UMP |
| −0.4514049 | 9.37E−33 | 7.02E−31 | CMP |
| −0.3782691 | 9.97E−23 | 1.72E−21 | GMP |
| −0.2890513 | 1.64E−13 | 9.20E−13 | oxalate |
| −0.2885142 | 1.82E−13 | 9.76E−13 | alpha-glycerophosphate |
| −0.2789947 | 1.18E−12 | 5.75E−12 | C46:0 TAG |
| −0.2787835 | 1.23E−12 | 5.86E−12 | C54:1 TAG |
| −0.2774658 | 1.58E−12 | 7.39E−12 | dCMP |
| −0.2701827 | 6.21E−12 | 2.85E−11 | C46:1 TAG |

TABLE 2
Unbiased analysis of MYC gene expression versus metabolite abundance
in 911 cell lines. Data obtained via public DepMap.org portal.
Rank ordered by Pearson's correlation metric (Cor).

| Cor | PValue | QValue | Metabolite |
|---|---|---|---|
| 0.27735512 | 1.49E−17 | 8.40E−16 | UMP |
| 0.26407235 | 5.33E−16 | 2.00E−14 | C54:1 TAG |
| 0.25585158 | 4.41E−15 | 1.24E−13 | AMP |
| 0.25491783 | 5.58E−15 | 1.40E−13 | C56:2 TAG |
| 0.25205034 | 1.14E−14 | 2.14E−13 | C54:2 TAG |
| 0.2480425 | 3.07E−14 | 4.93E−13 | alpha-glycerophosphate |
| 0.2475113 | 3.49E−14 | 5.24E−13 | CMP |
| 0.24620835 | 4.80E−14 | 6.74E−13 | 5-adenosylhomocysteine |
| 0.23935684 | 2.46E−13 | 3.07E−12 | GMP |
| 0.23067799 | 1.81E−12 | 1.94E−11 | C52:1 TAG |

TABLE 3
Half-maximal inhibitory concentrations (IC50) selective
DHODH inhibitors. Inhibitors were tested in limiting
dilution against different MB and NSC samples. Prestoblue
viability was used to readout the phenotype. IC50s
were calculated via nonlinear regression.

| | | | BAY2402234 | Brequinar |
|---|---|---|---|---|
| Cell Line | Type | Source | IC₅₀ (nM) | IC₅₀ (μM) |
| NSC197 | Neural | Patient | ND | ND |
| | Stem Cell | derived | | |
| HD-MB03 | G3MB- | Patient | 0.2429 | 0.05435 |
| | MYC | derived | | |
| SU_MB002 | G3MB- | Patient | 1.286 | 0.302 |
| | MYC | derived | | |
| D425 | G3MB- | Commercial | 0.4942 | 0.1221 |
| | MYC | | | |
| D458 | G3MB- | Commercial | 0.2595 | 0.1104 |
| | MYC | | | |
| MED411- | G3MB- | Patient | 0.6258 | 0.1369 |
| FHTC | MYC | derived | | |
| ICB1299 | G4MB | Patient | ND | |
| | | derived | | |
| MBT375 | G4MB | Patient | ND | |
| | | derived | | |

ND = not determined due to lack of dose-response.

TABLE 4A
Normalized peak intensities for metabolites identified from AAVS1 or DHODH
knockout SU_MB002 cells, or SU_MB002 cells treated with BAY (IC90) or its vehicle.
Significance determined via multiple t-tests.

| | AAVS1_{—} | AAVS1_{—} | DHODH_{—} | DHODH_{—} | BAY_{—} | BAY_{—} |
|---|---|---|---|---|---|---|
| Annotation | average | sd | average | sd | average | sd |
| TG(42:0) | 2172579.67 | 678480.866 | 9429396.1 | 4240059.53 | 15967351.9 | 3370312.28 |
| TG(44:0) | 10431070.9 | 3149457 | 39575101.4 | 11388292.9 | 64916660.2 | 6317278.67 |
| TG(44:1) | 8249500.85 | 2078394.89 | 56555837.9 | 24320161.6 | 72494555.5 | 7203274.46 |
| TG(44:2) | 1943606.19 | 348910.79 | 21071701.8 | 12392916.7 | 23582680.7 | 4284927.46 |
| TG(48:0) | 71634424.3 | 11973983.5 | 138873085 | 14827775 | 234715455 | 11904108 |
| TG(48:1) | 649848.325 | 186514.724 | 1329860.88 | 395355.164 | 2421262.9 | 318727.825 |
| TG(46:3) | 2744184.65 | 386994.744 | 33752101.4 | 19528360.7 | 35191113.7 | 6412305.65 |
| TG(48:3) | 3230032.58 | 791299.452 | 19196290.9 | 6694172.6 | 16895583.2 | 1574460.94 |
| TG(48:4) | 2965445.17 | 447945.163 | 34913574.2 | 18048178.3 | 33300539.1 | 5885900.32 |
| TG(48:5) | 177444.539 | 47151.6678 | 3189662.65 | 1888696.9 | 2707280.56 | 685930.664 |
| TG(50:0) | 87144057.5 | 13490774.4 | 127865627 | 17597278.3 | 235218307 | 16161390.5 |
| TG(50:2) | 497131847 | 84490548.1 | 1070068256 | 116170992 | 1182402177 | 61236623.2 |
| TG(50:3) | 171951528 | 32168149.8 | 592494541 | 127989297 | 595402718 | 46884211 |
| TG(52:5) | 3013571.11 | 803772.426 | 14557476.1 | 2768991.32 | 9440406.47 | 783760.325 |
| TG(52:1) | 368943615 | 52454209.4 | 479529542 | 69567279.8 | 649526618 | 35537783.3 |
| TG(54:1) | 64999630.4 | 9647789.69 | 83951115.1 | 10270997.9 | 104051454 | 3359263.85 |
| TG(56:7) | 7711505.17 | 1022985.88 | 26017456.1 | 4878391.26 | 13481295.5 | 1076162.47 |
| TG(54:6) | 15716460.7 | 1973897.04 | 61860889.4 | 11910476 | 36773026.8 | 2384848.44 |
| PE O-32:1 | 2006379.23 | 196305.758 | 7571684.47 | 605135.877 | 2472183.74 | 180499.906 |
| PE O-32:2 | 4916788.68 | 660090.194 | 15656205.9 | 647367.647 | 11446617.4 | 336108.317 |
| PE O-34:2 | 50498109 | 3791352.63 | 134325462 | 8340536.39 | 92503648 | 5557284.74 |
| PE O-36:1 | 358741.799 | 25395.2584 | 894327.322 | 57448.5486 | 531398.036 | 24952.0882 |
| PE O-36:4 | 130047745 | 12022391.6 | 239922409 | 14183034.5 | 258977958 | 10510978.6 |
| PE O-34:1 | 4393870.08 | 248313.775 | 11352655.2 | 722489.7 | 3839410.03 | 184859.696 |
| PE O-36:2 | 71148459 | 4776418.94 | 135241295 | 8166185.92 | 102597837 | 5500030.45 |
| PE O-38:4 | 130539026 | 9964331.38 | 180291455 | 19316710.5 | 210123122 | 14866429.8 |
| PE O-40:2 | 7965145.85 | 630699.375 | 11785750.9 | 1100816.21 | 10612336 | 838242.208 |
| PE O-40:4 | 172111027 | 8502935.02 | 223442242 | 17642277.8 | 226507622 | 10020301.9 |
| PE O-42:3 | 721585.137 | 62417.8712 | 1331708.75 | 124776.602 | 911901.571 | 45296.8086 |
| PE O-42:4 | 8332564.47 | 718208.489 | 14173788.8 | 1717662.23 | 13670542.5 | 800856.505 |
| PE O-42:5 | 14780848.9 | 695276.706 | 22651559.5 | 1758686.76 | 14681375.2 | 753388.95 |
| 2-Amino- | 69297 | 11663.7665 | 41417.7143 | 4245.72831 | 26487 | 888.010698 |
| adipic acid | | | | | | |
| alpha-Keto- | 259443.429 | 49858.5872 | 108084.625 | 21098.8418 | 112368 | 5129.24741 |
| glutaric | | | | | | |
| acid | | | | | | |
| Argino- | 60773.75 | 5065.99949 | 94872 | 6643.09729 | 114194 | 13346.5958 |
| succinic | | | | | | |
| acid | | | | | | |
| Asparagine | 815457.875 | 60902.8378 | 382564.625 | 99539.3778 | 172639.25 | 102222.882 |
| Aspartic Acid | 138725.5 | 12391.33 | 216932.714 | 10406.6062 | 214198 | 6066.76578 |
| CDP | 6323202 | 1192446.94 | 3511974.5 | 1381599.09 | 5476872.25 | 490593.241 |
| Citramalate | 8998.71429 | 1772.13635 | 4712.42857 | 395.043819 | 4459.5 | 569.718351 |
| Citrate | 513057.571 | 99114.8737 | 201661.875 | 60145.1818 | 76953 | 25521.4797 |
| Creatine | 1153441.38 | 151127.773 | 669777.25 | 156577.33 | 619707.25 | 63337.6586 |
| phosphate | | | | | | |
| CTP | 1082249.71 | 194508.731 | 554222.75 | 169902.243 | 810207.75 | 59700.2093 |
| Cystathionine | 15509.375 | 4914.06855 | 9994.25 | 2178.234 | 3636 | 564.368674 |
| dCTP | 22894.5 | 2562.53808 | 108002.333 | 2636.50804 | 109835 | 8124.77274 |
| Dihydro- | 1199808 | 461695.46 | 418701.375 | 105919.074 | 462202 | 36303.3257 |
| orotic Acid | | | | | | |
| dTDP | 2841522.75 | 436703.895 | 7915213.29 | 932328.428 | 13149248 | 2816460.69 |
| Gluconic acid | 725531.125 | 132829.356 | 726899.75 | 129124.96 | 1081576.5 | 142063.877 |
| Glutamate | 7581914.86 | 4132688.36 | 22594652.3 | 8821274.56 | 26227101.8 | 2652153.15 |
| Glyceralde- | 264016.25 | 24781.3646 | 170944.143 | 68204.4452 | 78313.3333 | 4727.42259 |
| hyde-3- | | | | | | |
| phosphate | | | | | | |
| Hydroxy- | 206917.25 | 30760.4925 | 30544382.1 | 3101750.25 | 37974496.3 | 5414958.59 |
| glutaric_{—} | | | | | | |
| acid | | | | | | |
| Inositol | 62696185.3 | 8626964.68 | 35065447.8 | 5289391.33 | 40859409.5 | 3905050.8 |
| N-Acetyl | 11190787 | 1348894.35 | 6686180 | 1351627.94 | 8697678.5 | 1107900.76 |
| glucosamine | | | | | | |
| mono- | | | | | | |
| phosphate | | | | | | |
| N-Acetyl- | 77206.7143 | 7286.83193 | 118250 | 8728.45964 | 189382 | 23353.3175 |
| glutamic | | | | | | |
| Acid | | | | | | |
| N-Carbamoyl | 184412.375 | 60212.181 | 43726.8571 | 3915.45401 | 31547.3333 | 3125.83322 |
| Aspartic | | | | | | |
| Acid | | | | | | |
| Proline | 57043.625 | 5218.19569 | 86267541.9 | 13648844.5 | 129729612 | 8392954.2 |
| Saccharopine | 30610.625 | 6589.78749 | 12384.1429 | 1806.67479 | 11126.3333 | 623.025949 |
| Sarcosine | 195590.143 | 22595.7802 | 152834.143 | 35314.7423 | 178498.5 | 16854.6877 |
| UDP | 5787900.38 | 387618.237 | 2895873 | 937458.306 | 964954.5 | 396797.368 |
| UDP-Glucose | 3080147.75 | 573347.186 | 1692351.5 | 727362.313 | 609888.333 | 17186.2408 |
| Uridine | 191164.714 | 98425.9733 | 74132.8571 | 54244.7367 | 26154.5 | 141748 |
| UTP | 1777816.71 | 387520.41 | 711695.5 | 223857.928 | 215065 | 17369.377 |
| Xanthosine | 29290.25 | 6583.02014 | 425982.571 | 113051.878 | 617698.75 | 119292.524 |

| | | | Log2 Fold | Log2 Fold | Adj. | Adj. |
|---|---|---|---|---|---|---|
| | | | Change: | Change: | P-value: | P-value: |
| | DMSO_{—} | DMSO_{—} | (DHODH)/ | (BAY)/ | (DHODH)/ | (BAY)/ |
| Annotation | average | sd | (AAVS1) | (DMSO) | (AAVS1) | (DMSO) |
| TG(42:0) | 2201006.23 | 606096.622 | 2.11775629 | 2.85888994 | 0.00010635 | 0.00013913 |
| TG(44:0) | 8417209.28 | 1202638.95 | 1.92370577 | 2.94717489 | 4.0732E−05 | 1.7667E−05 |
| TG(44:1) | 6112687.07 | 1589905.17 | 2.77729722 | 3.56799403 | 6.9135E−06 | 1.9797E−05 |
| TG(44:2) | 1209688.58 | 382089.151 | 3.43849899 | 4.28502013 | 1.2564E−05 | 3.2828E−05 |
| TG(48:0) | 66669545.2 | 4776142.04 | 0.95504206 | 1.81581305 | 0.01792981 | 0.00109341 |
| TG(48:1) | 415912.652 | 116651.63 | 1.03310039 | 2.54140726 | 0.01440437 | 2.2355E−05 |
| TG(46:3) | 1829805.35 | 480574.064 | 3.62052786 | 4.26544908 | 1.4594E−06 | 1.1372E−05 |
| TG(48:3) | 1928206.45 | 193247.12 | 2.57120696 | 3.13131472 | 1.0216E−08 | 1.4884E−07 |
| TG(48:4) | 2139408.62 | 679007.37 | 3.55746746 | 3.96026157 | 3.3588E−08 | 8.9971E−07 |
| TG(48:5) | 64619.9513 | 27943.5673 | 4.16796377 | 5.38872093 | 1.6241E−08 | 4.6241E−08 |
| TG(50:0) | 78979864.4 | 14003323.4 | 0.55315429 | 1.57444355 | 0.00119297 | 7.5412E−08 |
| TG(50:2) | 321055867 | 124578662 | 1.10600239 | 1.88082456 | 2.2627E−05 | 9.4084E−07 |
| TG(50:3) | 117135781 | 10080117.2 | 1.78479992 | 2.34568397 | 4.2551E−08 | 1.9542E−07 |
| TG(52:5) | 1765987.81 | 822502.252 | 2.27221423 | 2.41837359 | 1.2974E−07 | 5.0877E−06 |
| TG(52:1) | 310448684 | 20588101.3 | 0.37821935 | 1.06503383 | 0.02049117 | 6.9146E−06 |
| TG(54:1) | 62385846.4 | 4452463.41 | 0.36911797 | 0.73800646 | 0.00840226 | 0.00020055 |
| TG(56:7) | 5045045.07 | 650464.315 | 1.75439552 | 1.41802008 | 1.1739E−10 | 1.371E−06 |
| TG(54:6) | 10633750.7 | 821320.9 | 1.97675121 | 1.78999738 | 1.3425E−11 | 1.8504E−08 |
| PE O-32:1 | 955799.228 | 96692.8624 | 1.91601998 | 1.37100647 | 8.7181E−12 | 3.3669E−09 |
| PE O-32:2 | 3800964.61 | 407471.767 | 1.67094638 | 1.59048383 | 8.7181E−12 | 3.5516E−10 |
| PE O-34:2 | 37069497.1 | 2903977.97 | 1.41143153 | 1.31927772 | 8.7181E−12 | 2.5679E−11 |
| PE O-36:1 | 269120.856 | 34172.4626 | 1.3178571 | 0.98153869 | 8.7181E−12 | 1.0122E−08 |
| PE O-36:4 | 132626827 | 2968254.61 | 0.88352653 | 0.96545669 | 4.5137E−11 | 6.4597E−09 |
| PE O-34:1 | 2213542.65 | 129198.714 | 1.36946564 | 0.79452747 | 9.24E−12 | 6.4331E−06 |
| PE O-36:2 | 61280944.7 | 2806772.06 | 0.92663133 | 0.74348987 | 5.3403E−11 | 9.0186E−07 |
| PE O-38:4 | 134946384 | 2943005.44 | 0.46584984 | 0.63884861 | 6.5181E−05 | 0.00011853 |
| PE O-40:2 | 7649595.25 | 452845.575 | 0.565271 | 0.47228694 | 3.28E−07 | 0.00063663 |
| PE O-40:4 | 184389727 | 9164838.05 | 0.37656242 | 0.29680131 | 4.2074E−05 | 0.0263418 |
| PE O-42:3 | 635237.532 | 31087.6916 | 0.88403706 | 0.52158196 | 2.3592E−10 | 0.00018043 |
| PE O-42:4 | 8436410.37 | 514472.892 | 0.76639298 | 0.69636931 | 3.6567E−08 | 4.0262E−05 |
| PE O-42:5 | 12068413 | 564166.598 | 0.61588125 | 0.28275114 | 2.96E−08 | 0.04457171 |
| 2-Amino- | 94238.5 | 24753.4604 | −0.742545 | −1.8310321 | 0.005336 | 0.001393 |
| adipic acid | | | | | | |
| alpha-Keto- | 361916.75 | 42570.7824 | −1.2632587 | −1.6874266 | 0.00035 | 0.000107 |
| glutaric | | | | | | |
| acid | | | | | | |
| Argino- | 59627.75 | 3058.16965 | 0.64253405 | 0.93743105 | 0.000023 | 0.000223 |
| succinic | | | | | | |
| acid | | | | | | |
| Asparagine | 1012343.33 | 20002.429 | −1.0919069 | −2.5518663 | 0.00012 | 0.005477 |
| Aspartic Acid | 134821.333 | 1095.56256 | 0.64501463 | 0.66789621 | 0.000316 | 0.000067 |
| CDP | 11385905 | 1247502.14 | −0.8483729 | −1.0558248 | 0.008246 | 0.000223 |
| Citramalate | 9898.5 | 2948.73154 | −0.9332481 | −1.150328 | 0.00195 | 0.007675 |
| Citrate | 584027.333 | 29001.2609 | −1.3471824 | −2.9239864 | 0.00061 | 0.000478 |
| Creatine | 1081895.67 | 30185.5494 | −0.7841914 | −0.8039026 | 0.000567 | 0.000684 |
| phosphate | | | | | | |
| CTP | 2610965.33 | 72405.6484 | −0.9654956 | −1.6882195 | 0.000159 | 0.00002 |
| Cystathionine | 11884.5 | 746.219583 | −0.6339703 | −1.7086571 | 0.000522 | 0.000057 |
| dCTP | 22766.5 | 4018.49611 | 2.23798952 | 2.27035344 | 0.000055 | 0.000037 |
| Dihydro- | 2046664 | 345825.313 | −1.51881 | −2.1466789 | 0.00012 | 0.000217 |
| orotic Acid | | | | | | |
| dTDP | 3533261.5 | 225716.916 | 1.47796396 | 1.89590786 | 0.000235 | 0.000103 |
| Gluconic acid | 737440.75 | 83785.0625 | 1.57534719 | 2.80839655 | 0.000266 | 0.000007 |
| Glutamate | 3744024.67 | 103209.143 | 0.0027189 | 0.55253667 | 0.005505 | 0.009196 |
| Glyceralde- | 182905 | 4385.91598 | −0.6271017 | −1.2237647 | 0.000397 | 0.000115 |
| hyde-3- | | | | | | |
| phosphate | | | | | | |
| Hydroxy- | 256247.25 | 59576.3732 | 7.20570932 | 7.21135049 | <0.000001 | <0.000001 |
| glutaric_{—} | | | | | | |
| acid | | | | | | |
| Inositol | 69078844 | 529432.15 | −0.8383275 | −0.7575756 | 0.000397 | 0.000629 |
| N-Acetyl | 12573335 | 640553.312 | −0.7430574 | −0.5316651 | 0.005104 | 0.009858 |
| glucosamine | | | | | | |
| mono- | | | | | | |
| phosphate | | | | | | |
| N-Acetyl- | 81403.6667 | 1551.62893 | 0.61504196 | 1.21813353 | 0.000182 | 0.00029 |
| glutamic | | | | | | |
| Acid | | | | | | |
| N-Carbamoyl | 227957.75 | 7225.30236 | −2.0763439 | −2.8531765 | 0.000007 | 0.000004 |
| Aspartic | | | | | | |
| Acid | | | | | | |
| Proline | 52443.3333 | 4811.84999 | 10.5625365 | 11.2724608 | <0.000001 | <0.000001 |
| Saccharopine | 36455.25 | 3016.45668 | −1.3055385 | −1.7121484 | 0.00012 | 0.000037 |
| Sarcosine | 248347.333 | 8918.32239 | −0.3558668 | −0.4764473 | 0.0081 | 0.004669 |
| UDP | 7822006 | 386039.283 | −0.9990418 | −3.0190058 | 0.000079 | 0.000142 |
| UDP-Glucose | 4159628 | 239163.636 | −0.8639703 | −2.7698375 | 0.00035 | <0.000001 |
| Uridine | 429216.333 | 45976.2028 | −1.3666312 | −4.0365739 | 0.002858 | 0.000502 |
| UTP | 2230790.25 | 225381.339 | −1.3207746 | −3.3747102 | 0.000618 | <0.000001 |
| Xanthosine | 55128.75 | 15653.7628 | 3.86230199 | 3.48602662 | 0.000182 | 0.000059 |

TABLE 4B
Normalized peak intensities from flank tumor tissues of mice treated with BAY2402234
or its vehicle. Bolded values indicate those which were below the limit of detection.

| | | | | | N- | | | Uridine | | |
|---|---|---|---|---|---|---|---|---|---|---|
| | | dihydro- | | | Carbamoyl | | | 5′-diphos- | Uridine | |
| | BAY- | orotic | | | aspartic | Orotic | | phate | 5′-mono- | UDP- |
| Sample | 2402234 | acid | Glutamine | UTP | acid | acid | Uridine | (UDP) | phosphate | GluNAc |
| 17_BrainVeh_{—} | 2537 | 70960 | 417726240 | 26523 | 151732 | 1390954 | 56662708 | 106464 | 26678944 | 19240264 |
| 1 Area | | | | | | | | | | |
| 18_BrainVeh_{—} | 3147 | 62340 | 303306912 | 16630 | 147444 | 897676 | 40506864 | 61931 | 20195926 | 11022575 |
| 2 Area | | | | | | | | | | |
| 19_BrainVeh_{—} | 22497 | 46403 | 289349984 | 10149 | 157183 | 948583 | 44763824 | 34424 | 20644258 | 12582045 |
| 3 Area | | | | | | | | | | |
| 20_BrainVeh_{—} | 21976 | 45365 | 307084864 | 13375 | 121985 | 997462 | 29844462 | 80712 | 18945482 | 11499498 |
| 4 Area | | | | | | | | | | |
| 21_BrainBAY_{—} | 1338708 | 1975829 | 280983232 | 22962 | 1700352 | 3366687 | 41449420 | 133672 | 23218120 | 16715786 |
| 1 Area | | | | | | | | | | |
| 22_BrainBAY_{—} | 4796862 | 3553594 | 324326048 | 19870 | 1616900 | 6999989 | 113184456 | 97588 | 13372546 | 8363980 |
| 2 Area | | | | | | | | | | |
| 23_BrainBAY_{—} | 1717137 | 1415838 | 229454432 | 12207 | 1920988 | 4291824 | 51214976 | 69682 | 15616277 | 11501715 |
| 3 Area | | | | | | | | | | |
| 24_BrainBAY_{—} | 2579631 | 2677330 | 206007952 | 19991 | 2261730 | 3862066 | 33664876 | 73337 | 19018060 | 11534621 |
| 4 Area | | | | | | | | | | |
| 33_FlankVeh_{—} | 17507 | 113904 | 39829928 | 13979 | 296616 | 2101378 | 14814148 | 54305 | 97995120 | 33075048 |
| 1 Area | | | | | | | | | | |
| 34_FlankVeh_{—} | 5501 | 184134 | 74312032 | 4137 | 1309643 | 2607984 | 20520232 | 50897 | 56544980 | 54265604 |
| 2 Area | | | | | | | | | | |
| 35_FlankVeh_{—} | 43726 | 145557 | 73759080 | 519243 | 276482 | 6114253 | 11920890 | 595139 | 119461824 | 54242200 |
| 3 Area | | | | | | | | | | |
| 36_FlankBAY_{—} | 249608 | 119216720 | 55492788 | 4036 | 385157056 | 33617724 | 1736921 | 5654 | 17895140 | 2589539 |
| 1 Area | | | | | | | | | | |
| 37_FlankBAY_{—} | 266857 | 62149264 | 50587148 | 1148 | 380028608 | 35906864 | 2207031 | 1891 | 14218088 | 2049874 |
| 2 Area | | | | | | | | | | |
| 38_FlankBAY_{—} | 361684 | 89191448 | 68843176 | 5307 | 450759360 | 46970420 | 3920911 | 15124 | 25187528 | 3781049 |
| 3 Area | | | | | | | | | | |
| 39_FlankBAY_{—} | 146195 | 46399468 | 56207472 | 1474 | 384681824 | 34348420 | 1886173 | 5023 | 9202930 | 10999488 |
| 4 Area | | | | | | | | | | |

TABLE 5
Enrichment scores for pathways identified by gene set enrichment analysis
using normalized RNA sequencing data obtained from AAVS1 and DHODH knockout
SU_MB002 tumor cells. Pathways are ranked according to FDR.

| | | | | | | | RANK | |
|---|---|---|---|---|---|---|---|---|
| | | | | NOM | FDR | FWER | AT | LEADING |
| NAME | SIZE | ES | NES | p-val | q-val | p-val | MAX | EDGE |
| MYC TARGETS V1 | 196 | −0.59721 | −3.03358 | 0 | 0 | 0 | 3039 | tags = 65%, |
| | | | | | | | | list = 25%, |
| | | | | | | | | signal = 86% |
| MYC TARGETS V2 | 58 | −0.70132 | −2.7987 | 0 | 0 | 0 | 2133 | tags = 67%, |
| | | | | | | | | list = 18%, |
| | | | | | | | | signal = 81% |
| UNFOLDED | 105 | −0.42799 | −1.93309 | 0 | 6.67E−04 | 0.001 | 3232 | tags = 55%, |
| PROTEIN | | | | | | | | list = 27%, |
| RESPONSE | | | | | | | | signal = 75% |
| MTORC1 | 188 | −0.36347 | −1.83171 | 0 | 0.002586 | 0.006 | 2833 | tags = 42%, |
| SIGNALING | | | | | | | | list = 24%, |
| | | | | | | | | signal = 54% |
| OXIDATIVE | 176 | −0.35005 | −1.74316 | 0 | 0.005905 | 0.018 | 3367 | tags = 42%, |
| PHOSPHORYLATION | | | | | | | | list = 28%, |
| | | | | | | | | signal = 58% |
| NOTCH SIGNALING | 25 | −0.40285 | −1.40307 | 0.065934 | 0.062739 | 0.183 | 1517 | tags = 24%, |
| | | | | | | | | list = 13%, |
| | | | | | | | | signal = 27% |
| G2M CHECKPOINT | 186 | −0.25807 | −1.26284 | 0.018868 | 0.15344 | 0.446 | 3337 | tags = 39%, |
| | | | | | | | | list = 28%, |
| | | | | | | | | signal = 53% |
| FATTY ACID | 118 | −0.25885 | −1.22697 | 0.068493 | 0.169996 | 0.523 | 2436 | tags = 32%, |
| METABOLISM | | | | | | | | list = 20%, |
| | | | | | | | | signal = 40% |
| GLYCOLYSIS | 159 | −0.24806 | −1.21809 | 0.038462 | 0.160181 | 0.545 | 1725 | tags = 26%, |
| | | | | | | | | list = 14%, |
| | | | | | | | | signal = 30% |
| WNT BETA | 30 | −0.25223 | −0.89989 | 0.65873 | 0.8408 | 0.989 | 422 | tags = 7%, |
| CATENIN | | | | | | | | list = 4%, |
| SIGNALING | | | | | | | | signal = 7% |
| E2F TARGETS | 195 | −0.17252 | −0.88096 | 0.892857 | 0.806194 | 0.99 | 2818 | tags = 30%, |
| | | | | | | | | list = 24%, |
| | | | | | | | | signal = 39% |
| DNA REPAIR | 135 | −0.17518 | −0.84175 | 0.95946 | 0.810053 | 0.995 | 1232 | tags = 12%, |
| | | | | | | | | list = 10%, |
| | | | | | | | | signal = 13% |
| MYOGENESIS | 133 | 0.611789 | 2.206792 | 0 | 0 | 0 | 2307 | tags = 50%, |
| | | | | | | | | list = 19%, |
| | | | | | | | | signal = 61% |
| EPITHELIAL | 108 | 0.546527 | 1.934407 | 0 | 8.46E−04 | 0.002 | 2389 | tags = 47%, |
| MESENCHYMAL | | | | | | | | list = 20%, |
| TRANSITION | | | | | | | | signal = 59% |
| HALLMARK | 52 | 0.554918 | 1.781736 | 0 | 0.003526 | 0.012 | 3299 | tags = 54%, |
| INTERFERON | | | | | | | | list = 28%, |
| ALPHA RESPONSE | | | | | | | | signal = 74% |
| KRAS SIGNALING | 88 | 0.506893 | 1.739268 | 0 | 0.0046 | 0.02 | 1470 | tags = 31%, |
| DN | | | | | | | | list = 12%, |
| | | | | | | | | signal = 35% |
| APOPTOSIS | 110 | 0.486955 | 1.731383 | 0.001115 | 0.004528 | 0.024 | 2776 | tags = 38%, |
| | | | | | | | | list = 23%, |
| | | | | | | | | signal = 49% |
| TNFA SIGNALING | 119 | 0.43142 | 1.559441 | 0.002193 | 0.039095 | 0.234 | 2223 | tags = 35%, |
| VIA NFKB | | | | | | | | list = 19%, |
| | | | | | | | | signal = 43% |
| IL6 JAK STAT3 | 43 | 0.500328 | 1.554204 | 0.021223 | 0.035351 | 0.243 | 2170 | tags = 42%, |
| SIGNALING | | | | | | | | list = 18%, |
| | | | | | | | | signal = 51% |
| COAGULATION | 61 | 0.47203 | 1.550413 | 0.01699 | 0.032794 | 0.252 | 1870 | tags = 33%, |
| | | | | | | | | list = 16%, |
| | | | | | | | | signal = 39% |
| INTERFERON | 101 | 0.433806 | 1.51735 | 0.00453 | 0.043765 | 0.36 | 2911 | tags = 41%, |
| GAMMA RESPONSE | | | | | | | | list = 24%, |
| | | | | | | | | signal = 53% |
| ANGIOGENESIS | 19 | 0.558029 | 1.467132 | 0.060357 | 0.064788 | 0.537 | 1812 | tags = 37%, |
| | | | | | | | | list = 15%, |
| | | | | | | | | signal = 43% |
| APICAL JUNCTION | 137 | 0.402416 | 1.456283 | 0.008639 | 0.065317 | 0.578 | 2616 | tags = 31%, |
| | | | | | | | | list = 22%, |
| | | | | | | | | signal = 40% |
| P53 PATHWAY | 146 | 0.396707 | 1.439765 | 0.01507 | 0.072748 | 0.65 | 2083 | tags = 26%, |
| | | | | | | | | list = 17%, |
| | | | | | | | | signal = 31% |
| IL2 STAT5 | 127 | 0.39647 | 1.423199 | 0.021482 | 0.080005 | 0.705 | 1893 | tags = 29%, |
| SIGNALING | | | | | | | | list = 16%, |
| | | | | | | | | signal = 34% |
| APICAL SURFACE | 22 | 0.515263 | 1.417541 | 0.071014 | 0.078513 | 0.723 | 1932 | tags = 32%, |
| | | | | | | | | list = 16%, |
| | | | | | | | | signal = 38% |
| ESTROGEN | 132 | 0.395758 | 1.416631 | 0.013058 | 0.073864 | 0.726 | 2119 | tags = 27%, |
| RESPONSE EARLY | | | | | | | | list = 18%, |
| | | | | | | | | signal = 32% |
| UV RESPONSE UP | 121 | 0.375761 | 1.358787 | 0.040615 | 0.120555 | 0.9 | 2223 | tags = 27%, |
| | | | | | | | | list = 19%, |
| | | | | | | | | signal = 33% |
| INFLAMMATORY | 74 | 0.396438 | 1.349476 | 0.059929 | 0.121702 | 0.918 | 2495 | tags = 28%, |
| RESPONSE | | | | | | | | list = 21%, |
| | | | | | | | | signal = 36% |
| HYPOXIA | 148 | 0.366152 | 1.338856 | 0.034225 | 0.125841 | 0.936 | 1872 | tags = 26%, |
| | | | | | | | | list = 16%, |
| | | | | | | | | signal = 31% |
| KRAS SIGNALING | 99 | 0.378103 | 1.312053 | 0.079038 | 0.149986 | 0.966 | 2393 | tags = 30%, |
| UP | | | | | | | | list = 20%, |
| | | | | | | | | signal = 38% |
| UV RESPONSE DN | 112 | 0.352513 | 1.259028 | 0.096597 | 0.212244 | 0.991 | 2172 | tags = 24%, |
| | | | | | | | | list = 18%, |
| | | | | | | | | signal = 29% |
| ESTROGEN | 123 | 0.337564 | 1.219392 | 0.13895 | 0.269406 | 0.998 | 2214 | tags = 24%, |
| RESPONSE LATE | | | | | | | | list = 19%, |
| | | | | | | | | signal = 30% |
| PANCREAS BETA | 22 | 0.398809 | 1.086379 | 0.374131 | 0.567651 | 1 | 1188 | tags = 23%, |
| CELLS | | | | | | | | list = 10%, |
| | | | | | | | | signal = 25% |
| XENOBIOTIC | 123 | 0.299635 | 1.072382 | 0.348401 | 0.579796 | 1 | 2098 | tags = 23%, |
| METABOLISM | | | | | | | | list = 18%, |
| | | | | | | | | signal = 27% |
| HEDGEHOG | 26 | 0.382477 | 1.064938 | 0.373626 | 0.575378 | 1 | 1247 | tags = 19%, |
| SIGNALING | | | | | | | | list = 10%, |
| | | | | | | | | signal = 21% |
| CHOLESTEROL | 65 | 0.315687 | 1.056007 | 0.38835 | 0.576001 | 1 | 2348 | tags = 25%, |
| HOMEOSTASIS | | | | | | | | list = 20%, |
| | | | | | | | | signal = 30% |
| REACTIVE OXYGEN | 44 | 0.326981 | 1.039674 | 0.425031 | 0.596326 | 1 | 2526 | tags = 27%, |
| SPECIES PATHWAY | | | | | | | | list = 21%, |
| | | | | | | | | signal = 34% |
| COMPLEMENT | 111 | 0.294241 | 1.038555 | 0.403803 | 0.577311 | 1 | 2772 | tags = 31%, |
| | | | | | | | | list = 23%, |
| | | | | | | | | signal = 40% |
| TGF BETA | 41 | 0.336496 | 1.023196 | 0.445707 | 0.594457 | 1 | 2435 | tags = 24%, |
| SIGNALING | | | | | | | | list = 20%, |
| | | | | | | | | signal = 31% |
| PEROXISOME | 90 | 0.286894 | 0.990272 | 0.497738 | 0.652345 | 1 | 2688 | tags = 28%, |
| | | | | | | | | list = 23%, |
| | | | | | | | | signal = 36% |
| MITOTIC SPINDLE | 194 | 0.251233 | 0.935842 | 0.626819 | 0.757324 | 1 | 3763 | tags = 32%, |
| | | | | | | | | list = 32%, |
| | | | | | | | | signal = 46% |
| ADIPOGENESIS | 166 | 0.250554 | 0.927583 | 0.639327 | 0.751407 | 1 | 3174 | tags = 29%, |
| | | | | | | | | list = 27%, |
| | | | | | | | | signal = 39% |
| HEME | 146 | 0.235009 | 0.857565 | 0.750802 | 0.872759 | 1 | 3687 | tags = 36%, |
| METABOLISM | | | | | | | | list = 31%, |
| | | | | | | | | signal = 51% |
| BILE ACID | 70 | 0.24825 | 0.832932 | 0.742824 | 0.890833 | 1 | 2654 | tags = 27%, |
| METABOLISM | | | | | | | | list = 22%, |
| | | | | | | | | signal = 35% |
| ALLOGRAFT | 72 | 0.239823 | 0.816769 | 0.779326 | 0.893262 | 1 | 3060 | tags = 32%, |
| REJECTION | | | | | | | | list = 26%, |
| | | | | | | | | signal = 43% |
| ANDROGEN | 86 | 0.205685 | 0.70008 | 0.935484 | 1 | 1 | 1932 | tags = 14%, |
| RESPONSE | | | | | | | | list = 16%, |
| | | | | | | | | signal = 17% |
| PI3K AKT MTOR | 95 | 0.197784 | 0.684663 | 0.944954 | 1 | 1 | 2297 | tags = 16%, |
| SIGNALING | | | | | | | | list = 19%, |
| | | | | | | | | signal = 19% |
| SPERMATOGENESIS | 66 | 0.191812 | 0.635663 | 0.961631 | 1 | 1 | 3579 | tags = 30%, |
| | | | | | | | | list = 30%, |
| | | | | | | | | signal = 43% |
| PROTEIN | 88 | 0.146578 | 0.505357 | 1 | 0.998002 | 1 | 3414 | tags = 23%, |
| SECRETION | | | | | | | | list = 29%, |
| | | | | | | | | signal = 32% |

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### REFERENCES

1. Siegel, D. A., Richardson, L. C., Henley, S. J., Wilson, R. J., Dowling, N. F., Weir, H. K., Tai, E. W., and Buchanan Lunsford, N. (2020). Pediatric cancer mortality and survival in the United States, 2001-2016. Cancer 126, 4379-4389. https://doi.org/10.1002/cncr.33080.
2. Ostrom, Q. T., Patil, N., Cioffi, G., Waite, K., Kruchko, C., and Barnholtz-Sloan, J. S. (2020). CBTRUS Statistical Report: Primary Brain and Other Central Nervous System Tumors Diagnosed in the United States in 2013-2017. Neuro. Oncol. 22, iv1-iv96. 10.1093/neuonc/noaa200.
3. Ellison, D. W., Clifford, S. C., Gajjar, A., and Gilbertson, R. J. (2003). What's new in neuro-oncology? Recent advances in medulloblastoma. Eur. J. Paediatr. Neurol. 7, 53-66. 10.1016/s1090-3798 (03) 00014-x.
4. Hill, R. M., Richardson, S., Schwalbe, E. C., Hicks, D., Lindsey, J. C., Crosier, S., Rafiee, G., Grabovska, Y., Wharton, S. B., Jacques, T. S., et al. (2020). Time, pattern, and outcome of medulloblastoma relapse and their association with tumour biology at diagnosis and therapy: a multicentre cohort study. Lancet Child Adolesc. Heal. 4, 865-874. 10.1016/S2352-4642 (20) 30246-7.
5. Sabel, M., Fleischhack, G., Tippelt, S., Gustafsson, G., Doz, F., Kortmann, R., Massimino, M., Navajas, A., von Hoff, K., Rutkowski, S., et al. (2016). Relapse patterns and outcome after relapse in standard risk medulloblastoma: a report from the HIT-SIOP-PNET4 study. J. Neurooncol. 129, 515-524. 10.1007/s11060-016-2202-1.
6. Taylor, M. D., Northcott, P. A., Korshunov, A., Remke, M., Cho, Y. J., Clifford, S. C., Eberhart, C. G., Parsons, D. W., Rutkowski, S., Gajjar, A., et al. (2012). Molecular subgroups of medulloblastoma: The current consensus. Acta Neuropathol. 123, 465-472. 10.1007/s00401-011-0922-z.
7. Bao, S., Wu, Q., Mclendon, R. E., Hao, Y., Shi, Q., Hjelmeland, A. B., Dewhirst, M. W., Bigner, D. D., and Rich, J. N. (2006). Glioma stem cells promote radioresistance by preferential activation of the DNA damage response. Nature 444, 756-760. 10.1038/nature05236.
8. Nakai, E., Park, K., Yawata, T., Chihara, T., Kumazawa, A., Nakabayashi, H., and Shimizu, K. (2009). Enhanced MDR1 expression and chemoresistance of cancer stem cells derived from glioblastoma. Cancer Invest. 27, 901-908. 10.3109/07357900801946679.
9. Singh, S. K., Hawkins, C., Clarke, I. D., Squire, J. A., Bayani, J., Hide, T., Henkelman, R. M., Cusimano, M. D., and Dirks, P. B. (2004). Identification of human brain tumour initiating cells. Nature 432, 396-401. 10.1038/nature03031.1.
10. Singh, S. K., Clarke, I. D., Terasaki, M., Bonn, V. E., Hawkins, C., Squire, J., and Dirks, P. B. (2003). Identification of a Cancer Stem Cell in Human Brain Tumors. Cancer Res. 63, 5821-5828.
11. Bakhshinyan, D., Adile, A. A., Liu, J., Gwynne, W. D., Suk, Y., Custers, S., Burns, I., Singh, M., McFarlane, N., Subapanditha, M. K., et al. (2021). Temporal profiling of therapy resistance in human medulloblastoma identifies novel targetable drivers of recurrence. Sci. Adv. 7, eabi5568-eabi5568. 10.1126/sciadv.abi5568.
12. Liu, R., Li, W., Tao, B., Wang, X., Yang, Z., Zhang, Y., Wang, C., Liu, R., Gao, H., Liang, J., et al. (2019). Tyrosine phosphorylation activates 6-phosphogluconate dehydrogenase and promotes tumor growth and radiation resistance. Nat. Commun. 10, 991. 10.1038/s41467-019-08921-8.
13. Christian, S., Merz, C., Evans, L., Gradl, S., Seidel, H., Friberg, A., Eheim, A., Lejeune, P., Brzezinka, K., Zimmermann, K., et al. (2019). The novel dihydroorotate dehydrogenase (DHODH) inhibitor BAY 2402234 triggers differentiation and is effective in the treatment of myeloid malignancies. Leukemia 33, 2403-2415. 10.1038/s41375-019-0461-5.
14. Zhu, X., Chen, H.-H., Gao, C.-Y., Zhang, X.-X., Jiang, J.-X., Zhang, Y., Fang, J., Zhao, F., and Chen, Z.-G. (2020). Energy metabolism in cancer stem cells. World J. Stem Cells 12, 448-461. 10.4252/wjsc.v12.i6.448.
15. Sykes, D. B., Kfoury, Y. S., Mercier, F. E., Wawer, M. J., Law, J. M., Haynes, M. K., Lewis, T. A., Schajnovitz, A., Jain, E., Lee, D., et al. (2016). Inhibition of Dihydroorotate Dehydrogenase Overcomes Differentiation Blockade in Acute Myeloid Leukemia. Cell 167, 171-186.e15. 10.1016/j.cell.2016.08.057.
16. Pei, Y., Moore, C. E., Wang, J., Tewari, A. K., Eroshkin, A., Cho, Y.-J., Witt, H., Korshunov, A., Read, T.-A., Sun, J. L., et al. (2012). An animal model of MYC-driven medulloblastoma. Cancer Cell 21, 155-167. 10.1016/j.ccr.2011.12.021.
17. Reynolds, B. A., and Weiss, S. (1992). Generation of neurons and astrocytes from isolated cells of the adult mammalian central nervous system. Science (80-.). 255, 1707-1710. 10.1126/science. 1553558.
18. Toledo, C. M., Ding, Y., Hoellerbauer, P., Davis, R. J., Basom, R., Girard, E. J., Lee, E., Corrin, P., Hart, T., Bolouri, H., et al. (2015). Genome-wide CRISPR-Cas9 Screens Reveal Loss of Redundancy between PKMYT1 and WEE1 in Glioblastoma Stem-like Cells. Cell Rep. 13, 2425-2439. 10.1016/j.celrep.2015.11.021.
19. Ran, F. A., Hsu, P. D., Wright, J., Agarwala, V., Scott, D. A., and Zhang, F. (2013). Genome engineering using the CRISPR-Cas9 system. Nat. Protoc. 8, 2281-2308. 10.1038/nprot.2013.143\rhttp://www.nature.com/nprot/journal/v8/n11/abs/nprot.2013. 143.html#supplementary-information.
20. Hsu, P. D., Scott, D. a, Weinstein, J. a, Ran, F. A., Konermann, S., Agarwala, V., Li, Y., Fine, E. J., Wu, X., Shalem, O., et al. (2013). Genome-Scale CRISPR-Cas9 Knockout Screening in Human Cells. Nat. Biotechnol. 31, 827-832. 10.1038/nbt.2647.
21. Hart, T., Tong, A. H. Y., Chan, K., Van Leeuwen, J., Seetharaman, A., Aregger, M., Chandrashekhar, M., Hustedt, N., Seth, S., Noonan, A., et al. (2017). Evaluation and Design of Genome-Wide CRISPR/SpCas9 Knockout Screens. G3 (Bethesda). 7, 2719-2727. 10.1534/g3.117.041277.
22. Geiger, R., Rieckmann, J. C., Wolf, T., Basso, C., Feng, Y., Fuhrer, T., Kogadeeva, M., Picotti, P., Meissner, F., Mann, M., et al. (2016). L-Arginine Modulates T Cell Metabolism and Enhances Survival and Anti-tumor Activity. Cell 167, 829-842.e13. 10.1016/j.cell.2016.09.031.
23. Milde, T., Lodrini, M., Savelyeva, L., Korshunov, A., Kool, M., Brueckner, L. M., Antunes, A. S. L. M., Oehme, I., Pekrun, A., Pfister, S. M., et al. (2012). HD-MB03 is a novel Group 3 medulloblastoma model demonstrating sensitivity to histone deacetylase inhibitor treatment. J. Neurooncol. 110, 335-348. 10.1007/s11060-012-0978-1.
24. Lin, R., Elf, S., Shan, C., Kang, H.-B., Ji, Q., Zhou, L., Hitosugi, T., Zhang, L., Zhang, S., Seo, J. H., et al. (2015). 6-Phosphogluconate dehydrogenase links oxidative PPP, lipogenesis and tumour growth by inhibiting LKB1-AMPK signalling. Nat. Cell Biol. 17, 1484-1496. 10.1038/ncb3255.
25. Christian, S., Merz, C., Evans, L., Gradl, S., Seidel, H., Friberg, A., Eheim, A., Lejeune, P., Brzezinka, K., Zimmermann, K., et al. (2019). The novel dihydroorotate dehydrogenase (DHODH) inhibitor BAY 2402234 triggers differentiation and is effective in the treatment of myeloid malignancies. Leukemia 33, 2403-2415. 10.1038/s41375-019-0461-5.
26. Merve, A., Dubuc, A. M., Zhang, X., Remke, M., Baxter, P. A., Li, X.-N., Taylor, M. D., and Marino, S. (2014). Polycomb group gene BMI1 controls invasion of medulloblastoma cells and inhibits BMP-regulated cell adhesion. Acta Neuropathol. Commun. 2, 10. 10.1186/2051-5960-2-10.
27. Richards, L. M., Whitley, O. K. N., Macleod, G., Cavalli, F. M. G., Coutinho, F. J., Jaramillo, J. E., Svergun, N., Riverin, M., Croucher, D. C., Kushida, M., et al. (2021). Gradient of Developmental and Injury Response transcriptional states defines functional vulnerabilities underpinning glioblastoma heterogeneity. Nat. Cancer 2, 157-173. 10.1038/s43018-020-00154-9.
28. Tirosh, I., Venteicher, A. S., Hebert, C., Escalante, L. E., Patel, A. P., Yizhak, K., Fisher, J. M., Rodman, C., Mount, C., Filbin, M. G., et al. (2016). Single-cell RNA-seq supports a developmental hierarchy in human oligodendroglioma. Nature 539, 309-313. 10.1038/nature20123.
29. Neftel, C., Laffy, J., Filbin, M. G., Hara, T., Shore, M. E., Rahme, G. J., Richman, A. R., Silverbush, D., Shaw, M. L., Hebert, C. M., et al. (2019). An Integrative Model of Cellular States, Plasticity, and Genetics for Glioblastoma. Cell 178, 835-849.e21. 10.1016/j.cell.2019.06.024.
30. Filbin, M. G., Tirosh, I., Hovestadt, V., Shaw, M. L., Escalante, L. E., Mathewson, N. D., Neftel, C., Frank, N., Pelton, K., Hebert, C. M., et al. (2018). Developmental and oncogenic programs in H3K27M gliomas dissected by single-cell RNA-seq. Science 360, 331-335. 10.1126/science.aao4750.
31. Hovestadt, V., Smith, K. S., Bihannic, L., Filbin, M. G., Shaw, M. L., Baumgartner, A., DeWitt, J. C., Groves, A., Mayr, L., Weisman, H. R., et al. (2019). Resolving medulloblastoma cellular architecture by single-cell genomics. Nature 572, 74-79. 10.1038/s41586-019-1434-6.
32. Gulati, G. S., Sikandar, S. S., Wesche, D. J., Manjunath, A., Bharadwaj, A., Berger, M. J., Ilagan, F., Kuo, A. H., Hsieh, R. W., Cai, S., et al. (2020). Single-cell transcriptional diversity is a hallmark of developmental potential. Science 367, 405-411. 10.1126/science.aax0249.
33. Archer, T. C., Ehrenberger, T., Mundt, F., Gold, M. P., Krug, K., Mah, C. K., Mahoney, E. L., Daniel, C. J., LeNail, A., Ramamoorthy, D., et al. (2018). Proteomics, Post-translational Modifications, and Integrative Analyses Reveal Molecular Heterogeneity within Medulloblastoma Subgroups. Cancer Cell 34, 396-410.e8. 10.1016/j.ccell.2018.08.004.
34. Cavalli, F. M. G., Remke, M., Rampasek, L., Peacock, J., Shih, D. J. H., Luu, B., Garzia, L., Torchia, J., Nor, C., Morrissy, A. S., et al. (2017). Intertumoral Heterogeneity within Medulloblastoma Subgroups. Cancer Cell 31, 737-754.e6. 10.1016/j.ccell.2017.05.005.
35. Morrissy, A. S., Cavalli, F. M. G., Remke, M., Ramaswamy, V., Shih, D. J. H., Holgado, B. L., Farooq, H., Donovan, L. K., Garzia, L., Agnihotri, S., et al. (2017). Spatial heterogeneity in medulloblastoma. Nat. Genet. 49, 780-788. 10.1038/ng.3838.
36. Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Gene set enrichment analysis: A knowledge-based approach for interpreting genome-wide expression profiles. Proc. Natl. Acad. Sci. 102, 15545-15550. 10.1073/pnas.0506580102.
37. Maynard, R. E., Poore, B., Hanaford, A. R., Pham, K., James, M., Alt, J., Park, Y., Slusher, B. S., Tamayo, P., Mesirov, J., et al. (2021). TORC1/2 kinase inhibition depletes glutathione and synergizes with carboplatin to suppress the growth of MYC-driven medulloblastoma. Cancer Lett. 504, 137-145. https://doi.org/10.1016/j.canlet.2021.02.001.
38. Shin, S., Buel, G. R., Wolgamott, L., Plas, D. R., Asara, J. M., Blenis, J., and Yoon, S.-O. (2015). ERK2 Mediates Metabolic Stress Response to Regulate Cell Fate. Mol. Cell 59, 382-398. https://doi.org/10.1016/j.molcel.2015.06.020.
39. Zhang, W., Tong, H., Zhang, Z., Shao, S., Liu, D., Li, S., and Yan, Y. (2018). Transcription factor EGR1 promotes differentiation of bovine skeletal muscle satellite cells by regulating MyoG gene expression. J. Cell. Physiol. 233, 350-362. https://doi.org/10.1002/jcp.25883.
40. Ahmed, M. M., Sells, S. F., Venkatasubbarao, K., Fruitwala, S. M., Muthukkumar, S., Harp, C., Mohiuddin, M., and Rangnekar, V. M. (1997). Ionizing radiation-inducible apoptosis in the absence of p53 linked to transcription factor EGR-1. J. Biol. Chem. 272, 33056-33061. 10.1074/jbc.272.52.33056.
41. Chen, L., Wang, S., Zhou, Y., Wu, X., Entin, I., Epstein, J., Yaccoby, S., Xiong, W., Barlogie, B., Shaughnessy Jr, J. D., et al. (2010). Identification of early growth response protein 1 (EGR-1) as a novel target for JUN-induced apoptosis in multiple myeloma. Blood 115, 61-70. 10.1182/blood-2009-03-210526.
42. Sun, Z., Xu, X., He, J., Murray, A., Sun, M., Wei, X., Wang, X., McCoig, E., Xie, E., Jiang, X., et al. (2019). EGR1 recruits TET1 to shape the brain methylome during development and upon neuronal activity. Nat. Commun. 10, 3892. 10.1038/s41467-019-11905-3.
43. Kuleshov, M. V, Jones, M. R., Rouillard, A. D., Fernandez, N. F., Duan, Q., Wang, Z., Koplev, S., Jenkins, S. L., Jagodnik, K. M., Lachmann, A., et al. (2016). Enrichr: a comprehensive gene set enrichment analysis web server 2016 update. Nucleic Acids Res. 44, W90-7. 10.1093/nar/gkw377.
44. Itkonen, H. M., Minner, S., Guldvik, I. J., Sandmann, M. J., Tsourlakis, M. C., Berge, V., Svindland, A., Schlomm, T., and Mills, I. G. (2013). O-GlcNAc Transferase Integrates Metabolic Pathways to Regulate the Stability of c-MYC in Human Prostate Cancer Cells. Cancer Res. 73, 5277-5287. 10.1158/0008-5472.CAN-13-0549.
45. Lee, D. H., Kwon, N. E., Lee, W.-J., Lee, M.-S., Kim, D.-J., Kim, J. H., and Park, S.-K. (2020). Increased O-GlcNAcylation of c-Myc Promotes Pre-B Cell Proliferation. Cells 9, 158. 10.3390/cells9010158.
46. Le, T. T., Ziemba, A., Urasaki, Y., Hayes, E., Brotman, S., and Pizzorno, G. (2013). Disruption of uridine homeostasis links liver pyrimidine metabolism to lipid accumulation. J. Lipid Res. 54, 1044-1057. 10.1194/jlr.M034249.
47. Mao, C., Liu, X., Zhang, Y., Lei, G., Yan, Y., Lee, H., Koppula, P., Wu, S., Zhuang, L., Fang, B., et al. (2021). DHODH-mediated ferroptosis defence is a targetable vulnerability in cancer. Nature 593, 586-590. 10.1038/s41586-021-03539-7.
48. Di, L., Kerns, E. H., Fan, K., McConnell, O. J., and Carter, G. T. (2003). High throughput artificial membrane permeability assay for blood-brain barrier. Eur. J. Med. Chem. 38, 223-232. 10.1016/s0223-5234 (03) 00012-6.
49. Wang, X., Yang, K., Wu, Q., Kim, L. J. Y., Morton, A. R., Gimple, R. C., Prager, B. C., Shi, Y., Zhou, W., Bhargava, S., et al. (2019). Targeting pyrimidine synthesis accentuates molecular therapy response in glioblastoma stem cells. Sci. Transl. Med. 11. 10.1126/scitranslmed.aau4972.
50. Tanabe, A., and Sahara, H. (2020). The Metabolic Heterogeneity and Flexibility of Cancer Stem Cells. Cancers (Basel). 12, 2780. 10.3390/cancers12102780.
51. Intlekofer, A. M., and Finley, L. W. S. (2019). Metabolic signatures of cancer cells and stem cells. Nat. Metab. 1, 177-188. 10.1038/s42255-019-0032-0.
52. Kp, M., Kumar, A., Biswas, D., Moiyadi, A., Shetty, P., Gupta, T., Epari, S., Shirsat, N., and Srivastava, S. (2021). The proteomic analysis shows enrichment of RNA surveillance pathways in adult SHH and extensive metabolic reprogramming in Group 3 medulloblastomas. Brain Tumor Pathol. 38, 96-108. 10.1007/s10014-020-00391-x.
53. Shi, D. D., Savani, M. R., Levitt, M. M., Wang, A. C., Endress, J. E., Bird, C. E., Buehler, J., Stopka, S. A., Regan, M. S., Lin, Y.-F., et al. (2022). De novo pyrimidine synthesis is a targetable vulnerability in IDH mutant glioma. Cancer Cell 40, 939-956.e16. https://doi.org/10.1016/j.ccell.2022.07.011.
54. Pal, S., Kaplan, J. P., Nguyen, H., Stopka, S. A., Savani, M. R., Regan, M. S., Nguyen, Q.-D., Jones, K. L., Moreau, L. A., Peng, J., et al. (2022). A druggable addiction to de novo pyrimidine biosynthesis in diffuse midline glioma. Cancer Cell 40, 957-972.e10. https://doi.org/10.1016/j.ccell.2022.07.012.
55. Sodi, V. L., Khaku, S., Krutilina, R., Schwab, L. P., Vocadlo, D. J., Seagroves, T. N., and Reginato, M. J. (2015). mTOR/MYC Axis Regulates O-GlcNAc Transferase Expression and O-GlcNAcylation in Breast Cancer. Mol. Cancer Res. 13, 923-933. 10.1158/1541-7786.MCR-14-0536.
56. Saben, J., Zhong, Y., Gomez-Acevedo, H., Thakali, K. M., Borengasser, S. J., Andres, A., and Shankar, K. (2013). Early growth response protein-1 mediates lipotoxicity-associated placental inflammation: role in maternal obesity. Am. J. Physiol. Metab. 305, E1-E14. 10.1152/ajpendo.00076.2013.
57. Zou, Y., Henry, W. S., Ricq, E. L., Graham, E. T., Phadnis, V. V, Maretich, P., Paradkar, S., Boehnke, N., Deik, A. A., Reinhardt, F., et al. (2020). Plasticity of ether lipids promotes ferroptosis susceptibility and evasion. Nature 585, 603-608. 10.1038/s41586-020-2732-8.
58. Yun, S., Vincelette, N. D., Knorr, K. L. B., Almada, L. L., Schneider, P. A., Peterson, K. L., Flatten, K. S., Dai, H., Pratz, K. W., Hess, A. D., et al. (2016). 4EBP1/c-MYC/PUMA and NF-κB/EGR1/BIM pathways underlie cytotoxicity of mTOR dual inhibitors in malignant lymphoid cells. Blood 127, 2711-2722. 10.1182/blood-2015-02-629485.
59. Smith, K. S., Bihannic, L., Gudenas, B. L., Haldipur, P., Tao, R., Gao, Q., Li, Y., Aldinger, K. A., Iskusnykh, I. Y., Chizhikov, V. V, et al. (2022). Unified rhombic lip origins of group 3 and group 4 medulloblastoma. Nature 609, 1012-1020. 10.1038/s41586-022-05208-9.
60. Hendrikse, L. D., Haldipur, P., Saulnier, O., Millman, J., Sjoboen, A. H., Erickson, A. W., Ong, W., Gordon, V., Coudière-Morrison, L., Mercier, A. L., et al. (2022). Failure of human rhombic lip differentiation underlies medulloblastoma formation. Nature 609, 1021-1028. 10.1038/s41586-022-05215-w.
61. Edgar, R., Domrachev, M., and Lash, A. E. (2002). Gene Expression Omnibus: NCBI gene expression and hybridization array data repository. Nucleic Acids Res. 30, 207-210. 10.1093/nar/30.1.207.
62. Merve, A., Dubuc, A. M., Zhang, X., Remke, M., Baxter, P. A., Li, X.-N., Taylor, M. D., and Marino, S. (2014). Polycomb group gene BMI1 controls invasion of medulloblastoma cells and inhibits BMP-regulated cell adhesion. Acta Neuropathol. Commun. 2, 10. 10.1186/2051-5960-2-10.
63. Suk, Y., Kieliszek, A., Mobilio, D., Venugopal, C., and Singh, S. K. (2022). Derivation and culturing of neural stem cells from human embryonic brain tissue. STAR Protoc. 3, 101628. https://doi.org/10.1016/j.xpro.2022.101628.
64. Hart, T., Chandrashekhar, M., Aregger, M., Steinhart, Z., Brown, K. R., Macleod, G., Mis, M., Zimmermann, M., Fradet-Turcotte, A., Sun, S., et al. (2015). High-Resolution CRISPR Screens Reveal Fitness Genes and Genotype-Specific Cancer Liabilities. Cell 163, 1515-1526. 10.1016/j.cell.2015.11.015.
65. Dobin, A., Davis, C. A., Schlesinger, F., Drenkow, J., Zaleski, C., Jha, S., Batut, P., Chaisson, M., and Gingeras, T. R. (2012). STAR: ultrafast universal RNA-seq aligner. Bioinformatics 29, 15-21. 10.1093/bioinformatics/bts635.
66. Stuart, T., Butler, A., Hoffman, P., Hafemeister, C., Papalexi, E., Mauck, W. M. 3rd, Hao, Y., Stoeckius, M., Smibert, P., and Satija, R. (2019). Comprehensive Integration of Single-Cell Data. Cell 177, 1888-1902.e21. 10.1016/j.cell.2019.05.031.
67. Landry, A. P., Samuel, N., Spears, J., and Zador, Z. (2021). Integrated computational analyses reveal novel insights into the stromal microenvironment of SHH-subtype medulloblastoma. Sci. Rep. 11, 20694. 10.1038/s41598-021-00244-3.
68. Landry, A. P., Balas, M., Alli, S., Spears, J., and Zador, Z. (2020). Distinct regional ontogeny and activation of tumor associated macrophages in human glioblastoma. Sci. Rep. 10, 19542. 10.1038/s41598-020-76657-3.
69. McInnes, L., Healy, J., and Melville, J. (2018). UMAP: Uniform Manifold Approximation and Projection for Dimension Reduction.
70. Kanehisa, M., and Goto, S. (2000). KEGG: Kyoto Encyclopedia of Genes and Genomes. Nucleic Acids Res. 28, 27-30. 10.1093/nar/28.1.27.
71. Aibar, S., Gonzalez-Blas, C. B., Moerman, T., Huynh-Thu, V. A., Imrichova, H., Hulselmans, G., Rambow, F., Marine, J.-C., Geurts, P., Aerts, J., et al. (2017). SCENIC: single-cell regulatory network inference and clustering. Nat. Methods 14, 1083-1086. 10.1038/nmeth.4463.
72. Huynh-Thu, V. A., Irrthum, A., Wehenkel, L., and Geurts, P. (2010). Inferring Regulatory Networks from Expression Data Using Tree-Based Methods. PLOS One 5, 1-10. 10.1371/journal.pone.0012776.
73. Korotkevich, G., Sukhov, V., Budin, N., Shpak, B., Artyomov, M. N., and Sergushichev, A. (2021). Fast gene set enrichment analysis. bioRxiv. 10.1101/060012.
74. Merico, D., Isserlin, R., Stueker, O., Emili, A., and Bader, G. D. (2010). Enrichment Map: A Network-Based Method for Gene-Set Enrichment Visualization and Interpretation. PLOS One 5, 1-12. 10.1371/journal.pone.0013984.
75. Cavalcante, R. G., and Sartor, M. A. (2017). annotatr: genomic regions in context. Bioinformatics 33, 2381-2383. 10.1093/bioinformatics/btx183.
76. Aryee, M. J., Jaffe, A. E., Corrada-Bravo, H., Ladd-Acosta, C., Feinberg, A. P., Hansen, K. D., and Irizarry, R. A. (2014). Minfi: a flexible and comprehensive Bioconductor package for the analysis of Infinium DNA methylation microarrays. Bioinformatics 30, 1363-1369. 10.1093/bioinformatics/btu049.
77. Ritchie, M. E., Phipson, B., Wu, D., Hu, Y., Law, C. W., Shi, W., and Smyth, G. K. (2015). Limma powers differential expression analyses for RNA-sequencing and microarray studies. Nucleic Acids Res. 43, e47. 10.1093/nar/gkv007.
78. Nair A B, Jacob S. A simple practice guide for dose conversion between animals and human. J Basic Clin Pharm. 2016 March; 7 (2): 27-31. doi: 10.4103/0976-0105.177703. PMID: 27057123; PMCID: PMC4804402.

1. A method of treating a myc-amplified cancer comprising administering a dihydroorotate dehydrogenase (DHODH) inhibitor, to a subject in need thereof, wherein the myc-amplified cancer is not acute myeloid leukemia (AML).

2. The method of claim 1, wherein the myc-amplified cancer is brain cancer, such as MYC-amplified Group 3 medulloblastoma (G3MB), breast cancer, esophageal cancer, lymphoid cancer, myeloid cancer, lung cancer, kidney cancer, ovarian cancer, colorectal cancer or pancreatic cancer, optionally the myc-amplified cancer is a recurrent or a refractory cancer.

3. The method of claim 1, wherein the DHODH inhibitor is BAY2402234, Brequinar, PTC299, PTC868, or a combination thereof.

4. The method of claim 1, wherein the DHODH inhibitor is BAY2402234.

5. The method of claim 1, wherein the DHODH inhibitor is administered orally, or directly to the central nervous system, optionally directly to the brain.

6. The method of claim 1, wherein the DHODH inhibitor is administered at a dose of about 5 mg/kg/day to about 10 mg/kg/day.

7. The method of claim 1, wherein the DHODH inhibitor is administered as a pharmaceutical composition comprising the DHODH inhibitor and a pharmaceutically acceptable carrier or diluent.

8. The method of claim 1, wherein the DHODH inhibitor is administered as a combination therapy, optionally the combination therapy includes craniospinal irradiation and/or chemotherapy.

9. A method of treating a myc-amplified cancer in a subject comprising administering a dihydroorotate dehydrogenase (DHODH) inhibitor to a subject in need thereof, wherein the myc-amplified cancer has increased pyrimidine and/or purine metabolites relative to a non-cancerous control, wherein the cancer is not acute myeloid leukemia (AML).

10. The method of claim 9, further comprising, prior to administration,
a) obtaining a biopsy of the myc-amplified cancer; and
b) detecting increased pyrimidine and/or purine metabolites in the biopsy.

11. The method of claim 9, wherein the pyrimidine metabolites include cytidine-5-monophosphate (CMP), uridine-5-monophosphate (UMP) or both; and/or wherein the purine metabolites include adenosine-5-monophosphate (AMP), guanosine-5-monophosphate (GMP) or both.

12. The method of claim 9, wherein the myc-amplified cancer is brain cancer, such as MYC-amplified Group 3 medulloblastoma (G3MB), breast cancer, esophageal cancer, lymphoid cancer, myeloid cancer, lung cancer, kidney cancer, ovarian cancer, colorectal cancer or pancreatic cancer, optionally the myc-amplified cancer is a recurrent or a refractory cancer.

13. The method of claim 9, wherein the DHODH inhibitor is BAY2402234, Brequinar, PTC299, PTC868 or a combination thereof.

14. The method of claim 9, wherein the DHODH inhibitor is BAY2402234.

15. The method of claim 9, wherein the DHODH inhibitor is administered orally, or directly to the central nervous system, optionally directly to the brain.

16. The method of claim 9, wherein the DHODH inhibitor is administered at a dose of about 5 mg/kg/day to about 10 mg/kg/day.

17. The method of claim 9, wherein the DHODH inhibitor is administered as a pharmaceutical composition comprising the DHODH inhibitor and a pharmaceutically acceptable carrier or diluent.

18. The method of claim 9, wherein the DHODH inhibitor is administered as a combination therapy, optionally wherein the combination therapy includes craniospinal irradiation and/or chemotherapy.

19. A method of selecting a therapy for treating a myc-amplified cancer in a subject comprising:
a) obtaining a biopsy of the myc-amplified cancer;
b) detecting increased pyrimidine and/or purine metabolites in the biopsy; and
c) selecting a dihydroorotate dehydrogenase (DHODH) inhibitor for treating the subject when there is an increased level of pyrimidine and/or purine metabolites in the biopsy, wherein the cancer is not acute myeloid leukemia (AML).

20. The method of claim 19, wherein the pyrimidine metabolites include cytidine-5-monophosphate (CMP), uridine-5-monophosphate (UMP) or both and/or the purine metabolites include adenosine-5-monophosphate (AMP), guanosine-5-monophosphate (GMP) or both.

21. The method of claim 19, wherein the myc-amplified cancer is brain cancer, such as MYC-amplified Group 3 medulloblastoma (G3MB), breast cancer, esophageal cancer, lymphoid cancer, myeloid cancer, lung cancer, kidney cancer, ovarian cancer, colorectal cancer or pancreatic cancer, optionally the myc-amplified cancer is a recurrent or a refractory cancer.

22. The method of claim 19, wherein the DHODH inhibitor is BAY2402234, Brequinar, PTC299, PTC868, or a combination thereof.

23. The method of claim 19, wherein the DHODH inhibitor is BAY2402234.
